SEQUENCE LISTING (218 sequences)

SEQ ID NO: 1 - DNA, 101 bp, Homo sapiens
tgtcctttcttagcagagcaggattttgacctaaatttctgcaaactatartcttatggttatgactctttttgtaagttgatcactcattcacaaggatg

SEQ ID NO: 2 - DNA, 101 bp, Homo sapiens
aataagccccctcccactacttcccatttatgaaatctgtggcatactacvttactattttctatgaacctttcctggatcacttaacatgtttactacaa

SEQ ID NO: 3 - DNA, 101 bp, Homo sapiens
ggcggggcaggcggcggcgctgtcgggcgggcaggggtggcgggaggcggbggcgcagcgagcagcggcctccagcgctggtggctccctttataggagcg

SEQ ID NO: 4 - DNA, 101 bp, Homo sapiens
ttgaattcgggcgtctgctggagtgtgcccaatgctatatgtcagttgagrttctaagacttggaagccacagaaatgcagaatgccactctgaggataca

SEQ ID NO: 5 - DNA, 101 bp, Homo sapiens
agggcaggtcggggaggctgtgcttctgcctggagcccagataccccaaarccggagccagctgcctgctggtgctgaagacgagaaagcacagcccggtc

SEQ ID NO: 6 - DNA, 101 bp, Homo sapiens
gggagatgtggacagctccggcagctcgtgttcgcagtcaccctgaacgcyctcctctgaactcccacgtgtctccattctcctaagctcaggtcgtcaaa

SEQ ID NO: 7 - DNA, 101 bp, Homo sapiens
ccttcccgggctggggccagtgggccctgcctaggggagatgtggacagcbccggcagctcgtgttcgcagtcaccctgaacgccctcctctgaactccca

SEQ ID NO: 8 - DNA, 101 bp, Homo sapiens
actgcaactgccaagcagccggggtaggaggggcgccctaggcacagctgrgcccttgagacagcagggcttcgatgtcaggctcgatgtcaatggtctgg

SEQ ID NO: 9 - DNA, 101 bp, Homo sapiens
gcggcctgaaggatgctgataaccgggagccccgccctgggttcggctatyccgggcaccccgggccggcggggcgaggctctccaattgctgggccagag

SEQ ID NO: 10 - DNA, 101 bp, Homo sapiens
gaaactaaggataagtctcccctctcccctgaatttcaagatacctgtgcrgttatcaatatgtaaataaatgtaatttgaaagtcactttaaagattact

SEQ ID NO: 11 - DNA, 101 bp, Homo sapiens
tacttctaatacattttattggcacaaaattgtcacactggccttacctaraggtagaggactaggaaatatagcttaaccctgtgctcagggagaagaaa

SEQ ID NO: 12 - DNA, 101 bp, Homo sapiens
tatattgtgcctactgtgtgccaggcactatatttagcactttatatataytaactgcagctggccttcaagttggatttttttttttttaggtcattcct

SEQ ID NO: 13 - DNA, 101 bp, Homo sapiens
caattccatggcccatagagttacccttttccatatgcctttgaaatgccrgagatatttgatcagtcagtgtccctcctttcatgtgcaccccctgccag

SEQ ID NO: 14 - DNA, 101 bp, Homo sapiens
ttacgtgaatggaagcaaagtcaaggcaagtgtcaaggatgtgttgaaaamcagatattcaaaatggtgggcaaaactatgcaaatgacaagggcaatgct

SEQ ID NO: 15 - DNA, 101 bp, Homo sapiens
taagattccacttgtgagcaaggagaccatatacagtgccttctcccagakcagaacatacagagaaaaaaacaactgcctaatctgggaaggtgagatta

SEQ ID NO: 16 - DNA, 101 bp, Homo sapiens
gggatagaaccaattgtattcagtgagggccaagaaaattgtaatgctgtrcccactaaacaaaaaccatctgggagccagattcacactagggtggccag

SEQ ID NO: 17 - DNA, 101 bp, Homo sapiens
cttgtagtgactgagacacagtgacattatatcacaacctcagaaaccacracataaaccaaggaataatcaatgccatagtttttaatagtgcaactaga

SEQ ID NO: 18 - DNA, 101 bp, Homo sapiens
agagagaaagacttacaagaataaagtgaggaaaacacggagttgatgcasaagccccaacatccaacctcgactcctctttcgtagatgagaaactctgt

SEQ ID NO: 19 - DNA, 101 bp, Homo sapiens
ctgtcgcccaggctggagtgcagtggcgtgatctcggctcactgcaagctmcgcctcccacgttcacgccattctcctgcctcagcctcccgagtagctgg

SEQ ID NO: 20 - DNA, 101 bp, Homo sapiens
gccgtggcccccttctccagtgctctcagggagggtgcaccaggcctgccyccgccgtgagaaactgcagtccccttctccagtgctctcggggagggtgc

SEQ ID NO: 21 - DNA, 101 bp, Homo sapiens
tcccacattttaccctgtgaggaaatcgaggctcagaaaggctgagtggcrtgctcagggcatcagctcgtagggactgagccagggttggagtccagact

SEQ ID NO: 22 - DNA, 101 bp, Homo sapiens
tcaatatttggatttagtcttccctttatagaaaataaggacatgttgtaytgtattcttgcacactgaagtctgggggctacgattcattcagctcattg

SEQ ID NO: 23 - DNA, 101 bp, Homo sapiens
  modified_base (51)..(51): a, c, t or g
tcctgggcgccatcgccgtggaccgctacatctccatcttctacgcactgngctaccacagcatcgtgaccctgccgcgggcgcggcgagccgttgcggcc

SEQ ID NO: 24 - DNA, 101 bp, Homo sapiens
agttcccagttctcctcctgcctccggagctgagtgatggctgtgcttctstgacagtgtgaccctcacattagtcaacaataaacaacaaaaactgccac

SEQ ID NO: 25 - DNA, 101 bp, Homo sapiens
tatggtagaagagagaggagggtttctgtgttatgaactgcacgagttggytgagctcagtctatcacgtgtgtggtgggcacatggccagactccatgtg

SEQ ID NO: 26 - DNA, 101 bp, Homo sapiens
cagccctgtggttgatataaggaggagcagagagccaggtggggctgcagytctgtttctgggggaggtgggctcagaggtggctggggcttttctttaag

SEQ ID NO: 27 - DNA, 101 bp, Homo sapiens; identical to SEQ ID NO: 18

SEQ ID NO: 28 - DNA, 101 bp, Homo sapiens
tcttagtctgaataaccttttcctctgcagtatttttgagcagtggctccraaggcaccgtcctcttcaagaagtttatccagaagccaatgcacccattg

SEQ ID NO: 29 - DNA, 101 bp, Homo sapiens; identical to SEQ ID NO: 21

SEQ ID NO: 30 - DNA, 101 bp, Homo sapiens
atcctagacttaatttatcaaaggaatcccatgacttccaggaatagccakgcactactcagtaattaaacaggagcagcctgtggaagaaaggacttcat

SEQ ID NO: 31 - DNA, 101 bp, Homo sapiens; identical to SEQ ID NO: 23
  modified_base (51)..(51): a, c, t or g

SEQ ID NO: 32 - DNA, 101 bp, Homo sapiens
atttctagaccgatgactgcatataaagcaatgcttgagtgaagaaaacartagagtaggtagaaatggacatcgatatagagaatttgatactgatggat

SEQ ID NO: 33 - DNA, 101 bp, Homo sapiens; identical to SEQ ID NO: 28

SEQ ID NO: 34 - DNA, 101 bp, Homo sapiens
tactttttagctgtgtgaccttagataaattattaaacctttctgagcttyagttacctcttttttatctacaaaatggagataataagacatacctttta

SEQ ID NO: 35 - DNA, 101 bp, Homo sapiens; identical to SEQ ID NO: 21

SEQ ID NO: 36 - DNA, 101 bp, Homo sapiens; identical to SEQ ID NO: 24

SEQ ID NO: 37 - DNA, 101 bp, Homo sapiens
aagcaaacggaacgatgcttccctcaactcacttctgggaaaacaattcamagcacacagtggcagttcttgtttttaaacaaagtggagctgagagaggt

SEQ ID NO: 38 - DNA, 101 bp, Homo sapiens; identical to SEQ ID NO: 15

SEQ ID NO: 39 - DNA, 101 bp, Homo sapiens
gctgagcagaggatgaacatagccttggtcggatccctttatgagtcagayggttttcttcctgtgaggtgggtcctcagtgggagggactagagacagga

SEQ ID NO: 40 - DNA, 101 bp, Homo sapiens; identical to SEQ ID NO: 13

SEQ ID NO: 41 - DNA, 101 bp, Homo sapiens
tgagcttcagtttcttaaaatttaaaatgaggacaataccatctatggccrgggattaaatgctatgaggaatgtaaaccagatgtcaggtaccatctctc

SEQ ID NO: 42 - DNA, 101 bp, Homo sapiens; identical to SEQ ID NO: 16

SEQ ID NO: 43 - DNA, 101 bp, Homo sapiens; identical to SEQ ID NO: 11

SEQ ID NO: 44 - DNA, 101 bp, Homo sapiens; identical to SEQ ID NO: 10

SEQ ID NO: 45 - DNA, 101 bp, Homo sapiens; identical to SEQ ID NO: 32

SEQ ID NO: 46 - DNA, 101 bp, Homo sapiens; identical to SEQ ID NO: 28

SEQ ID NO: 47 - DNA, 101 bp, Homo sapiens; identical to SEQ ID NO: 23
  modified_base (51)..(51): a, c, t or g

SEQ ID NO: 48 - DNA, 101 bp, Homo sapiens
acaagaacgaaacagagttcaatggtctaaatttgcattcacgtgcagggytcctagaaatgatgatcctgcataattgttgtggaaatcatttgtcttct

SEQ ID NO: 49 - DNA, 101 bp, Homo sapiens
aaaatgaactttgtgatgtcttttctctatatttttggttgggaggagtarctagaattcctctcctaaattagcattgaatagcattctgtagaatatta

SEQ ID NO: 50 - DNA, 101 bp, Homo sapiens
aaactagatcagtggttacctggcagaatgttgggtgagggaaggtctccrgatcgggagggaagtaaatgaggatgggattacaaagggacacaaagaga

SEQ ID NO: 51 - DNA, 101 bp, Homo sapiens
acaggagaagggaggggaagggcagaagtccacagctgggagcacagggaytcgggtgacttatgctggggcctatttctcgttcatccctacaactggct

SEQ ID NO: 52 - DNA, 101 bp, Homo sapiens
gctataagaggggcgggcaggcatggagccccgtaggaatcgcagcgccarcggttgcaaggtaaggccccggcgcgctccttcctccttctctgctggtc

SEQ ID NO: 53 - DNA, 101 bp, Homo sapiens
ctttaatgactacaacattatagaagtttaaagcaggagagattgtatccygatggaaatgacaagaaaagcttcagggggaaggtgacatttaagttgga

SEQ ID NO: 54 - DNA, 101 bp, Homo sapiens
aagttaggctaccagcatatttgaatgccaggtgaaatcaaaataatctayactatctagaagactttcttgatgccaagtccagagatgtcattgtgtag

SEQ ID NO: 55 - DNA, 101 bp, Homo sapiens
tttaaaccaaccaccagatatctaaagagggaatacagcacagtgttggaragaaagtacagaatagtatttgagatcctagatgcagccggacgcggtgg

SEQ ID NO: 56 - DNA, 101 bp, Homo sapiens
gcaatgtccttgtttgtgttctctcccagtgttccaggttctactgtcaayccaggctcaggctgtcccacatcctcccacagaggtcttgctttgttttg

SEQ ID NO: 57 - DNA, 101 bp, Homo sapiens
tgagaggaattcaatctgaacaaatttaagcaaaagggatctttagtatgyggattttgtcattttctagtagacaccaaggacagggctgtagtggggcc

SEQ ID NO: 58 - DNA, 101 bp, Homo sapiens
agaggatgctaggtatctcaaggtaggaaagcatatctgtggacagaaagractgtagaatagccaaatcagagggaagggccactctacctagttcagtg

SEQ ID NO: 59 - DNA, 101 bp, Homo sapiens
aactgctatgtgtcctaagtgggaatgctaacccctctgatcggctgagaygcctacagcccagccttctctaaatccccaaaggccagaccctgaaatga

SEQ ID NO: 60 - DNA, 101 bp, Homo sapiens
tcaccaatatattattttacttatcagtgaaatcaaaggactttacatatytagattccaaaacaacctattgtgataatttcttacctagaaaggtttct

SEQ ID NO: 61 - DNA, 101 bp, Homo sapiens
ttagaatgtctgaattattattctaggttccttgcgactgctgtgaatttwgtgatgcacttggatagtctctgttactctaaagttttaataggtaacag

SEQ ID NO: 62 - DNA, 101 bp, Homo sapiens
tgccagcttcatagcctagtctaggcatgccagttgcccactgtggcaatmaatatctgagcctgtggtttttgccttaggtaaactgtagagatggactc

SEQ ID NO: 63 - DNA, 101 bp, Homo sapiens
agcactggcttagaagatgtaggcagagatgacaagtgacacttcctgtcrtctgcctacaagttcccaaagatcctcccctttcttgctctgttttcacc

SEQ ID NO: 64 - DNA, 101 bp, Homo sapiens
ataagctttctgcctcaatctatctgtgtaaggaacagggtttctctgaargtatctttgaaatactctaccatcagttcatatttctactttcacctaag

SEQ ID NO: 65 - DNA, 101 bp, Homo sapiens
cggttctaagcaacagatactgatactgactcttaccaaacaaagcatgarcaaacaaagatttatcagaagggtgcttgttagtacctgtattcaaaggg

SEQ ID NO: 66 - DNA, 101 bp, Homo sapiens
tttacagcatgatgaaattacatatatgatggttaggttaggttgcaagtyttggaatatatgcagaggaataactttattacaatgactatttacttttt

SEQ ID NO: 67 - DNA, 101 bp, Homo sapiens
tagacaagcagagccctgccaggcccatggtgacctctgcagacctaggarctgcaggcagagttgggggctcgttcctggggaggggcccacccctgagg

SEQ ID NO: 68 - DNA, 101 bp, Homo sapiens
aggtagtttggagaattgttcattactgaaatcactgtccctcagttcacyggtcttgtctgcttcgtcgtcaaaaacagcttgactgggatgaccgaagt

SEQ ID NO: 69 - DNA, 101 bp, Homo sapiens
acaatcactccttaaatatggtggaacacttgaagcttgatatctagtttbgattcaaaagcttcatttcccatattatgcaaaactggtggttgtgatct

SEQ ID NO: 70 - DNA, 101 bp, Homo sapiens; identical to SEQ ID NO: 54

SEQ ID NO: 71 - DNA, 101 bp, Homo sapiens; identical to SEQ ID NO: 59

SEQ ID NO: 72 - DNA, 101 bp, Homo sapiens
actgtatccattatattctcatcaccatcacatgtggttgaacgggcttcygactaaagaatctaaacatgtttaaaacatttttcacctccagtaaaact

SEQ ID NO: 73 - DNA, 101 bp, Homo sapiens; identical to SEQ ID NO: 54

SEQ ID NO: 74 - DNA, 101 bp, Homo sapiens; identical to SEQ ID NO: 59

SEQ ID NO: 75 - DNA, 101 bp, Homo sapiens; identical to SEQ ID NO: 72

SEQ ID NO: 76 - DNA, 101 bp, Homo sapiens
cctatacacttctatgtgtcttttcttatttctgtgctgcaaccaggtggyataacctctcacctgattccttagctctagtgaagttattttcgtgcatg

SEQ ID NO: 77 - DNA, 101 bp, Homo sapiens
atttttgctggaattataaagctagaggccttctctttccatggaggttgycacattcctaacaaatgagcctggagctgctggcagccatctttaacatc

SEQ ID NO: 78 - DNA, 101 bp, Homo sapiens
gattagttatgagcatactttggcaaatctctgcccctttgggctgcagcmtcacaagctgtgtggcgttgggcaagtctatagaactcaggacaaatggg

SEQ ID NO: 79 - DNA, 101 bp, Homo sapiens
cagcaaccatccacagagacatcctggagcctgggaaggagaaggacaaavagcccccttttttaaattttttttatgtttttgagacggagtctcactct

SEQ ID NO: 80 - DNA, 101 bp, Homo sapiens
ccctacccccaccctccatcccctggtgccctggggggatttattggagtrtatcaacctctccaacagcccctctaagagtcaggcttcaaagggtcctt

SEQ ID NO: 81 - DNA, 101 bp, Homo sapiens
ggagcagcagagtctgatgttgtgtacttcagggagctggagttctatgarggaagagcgaggaggcatgtgggaggaagaacagccccactgaggcctgc

SEQ ID NO: 82 - DNA, 101 bp, Homo sapiens
atgagaaacagcgtcaaatcatcttttcatgatcccaagctgaaaggcaakccctccagagagcgttatgtgacccacaaccgagcacattggtgacagac

SEQ ID NO: 83 - DNA, 101 bp, Homo sapiens
tcttccctggaggggctgttttcactgtgatgcccgcaacataccaagagyggaatcctgtctgaggagtgcagctccggtctcaccatgtgggcagggca

SEQ ID NO: 84 - DNA, 101 bp, Homo sapiens
agatgctgtcatgaagtcctagatagtcatcactttctaacaaggccctaygctgaacttaatctctgtaagtggcagaggcatttgaaacagagggctgc

SEQ ID NO: 85 - DNA, 101 bp, Homo sapiens
aaagtagaaaaattgtaagtagaattatcgttcattggggactgtctatamctcattagatgttctcagtcacagcctctattttatgaataattgtttta

SEQ ID NO: 86 - DNA, 101 bp, Homo sapiens
ctctgagatgtgctggcttcaggcaccaggtcggccacacactggagtagrgcaccaaacaggttcttgaagtccccaattttaggccttggttcttggat

SEQ ID NO: 87 - DNA, 101 bp, Homo sapiens
aaacctttaacttaaaattagaagcaagtctgatcaagaagtctcaagcamaggctgagtagtaatatttaagacaacactgcttactaaagaaaagagtt

SEQ ID NO: 88 - DNA, 101 bp, Homo sapiens
tttctattccaacgttccttggttattctgacttgtttgagaggaatgtayagatgatttttattttgccgcagggctgtcaatgcttttggttcacttag

SEQ ID NO: 89 - DNA, 101 bp, Homo sapiens
gtgttttaccatacttcaatttgttttcattgtgtttttggcttatctgtracagcttttcaatcagcttcctttaattgaggacttgacttggtttctaa

SEQ ID NO: 90 - DNA, 101 bp, Homo sapiens
ctttattttgctactgccttgacctcaaaggaatgtgatagtgtgaggtaygaatgctcttaataaacaggatcgatcaagggtgcttgactcttgttgtt

SEQ ID NO: 91 - DNA, 101 bp, Homo sapiens
gggcaatggtcccattttaaaatatgctgtcccattgtcccctagagcctrctttaacttgtcagaccatgtattccacttcatatgcaagaggcatgcac

SEQ ID NO: 92 - DNA, 101 bp, Homo sapiens
tcaataaagcaactatacaatcaagaaatgcaacacagatacctaataacvacacggcaagaaaaaaacctaacatatcaatattaatcttgaacataaac

SEQ ID NO: 93 - DNA, 101 bp, Homo sapiens
tggttatccagactcactcatcttcagcttctcaggggtccaatcctgcartatctagtgccactgctcctttcttccattcccattggcaccccccagcc

SEQ ID NO: 94 - DNA, 101 bp, Homo sapiens
tcccgagcagcggttgctgccgcccaccagcctcaggccccatgttgcatyacctgcaccaagaacaatgaagcaagtagttaatgggtgtgatggtttat

SEQ ID NO: 95 - DNA, 101 bp, Homo sapiens
atggaattcttcctatggtgtgaatgattccttcccagatggagactatgrtgccaacctggaagcagctgccccctgccactcctgtaacctgctggatg

SEQ ID NO: 96 - DNA, 101 bp, Homo sapiens
gaggaatgagaaggcagcaatctttgctgttctgcagccttcgctggtaayacccaggcaaatagggtctggagtggacctccatcaaactgcagcagaat

SEQ ID NO: 97 - DNA, 101 bp, Homo sapiens
ctatgtaaaattttagaatcagctgtcaactttacaaaaatttcttctggrgttttaagtgagattatgtggactctgtagatccatctggggagaagtga

SEQ ID NO: 98 - DNA, 101 bp, Homo sapiens
tatgtttaacagcagcatgaaaacagactaatatagtaaatttctgccagkggagtggggcattgcttagaagatacccaaaaatgtagaagtgagtttgg

SEQ ID NO: 99 - DNA, 101 bp, Homo sapiens
agaattaataccatgaaaagggggcagttcactcaacaaatatactgatargaaacagaatataagagccaatagagaagttttttgttgagaagtataat

SEQ ID NO: 100 - DNA, 101 bp, Homo sapiens
ttcttggttatgctccccgacctgttccaccacaaacacatgacaaaactytgagatatagatctagaaagtcctcacagccatctgatcaactgcagaaa

SEQ ID NO: 101 - DNA, 101 bp, Homo sapiens
ccttccatttgcaagaagcactagtaattttacacgaggggtgaccatctscacggtcattattgcaggagctcagcggcatccacagctgcaggggccca

SEQ ID NO: 102 - DNA, 101 bp, Homo sapiens
gagaagggagatggcggtagcagcgacgtgcccacctgtgatttctggggbccttcttttctctttgctggttcagggactcaagtccaggccaatttgac

SEQ ID NO: 103 - DNA, 101 bp, Homo sapiens
ttgttgtggcaactgggccagtgggacaggaaaggcgtcctgaagctctcrgctgggaagctcctgaagttgctctgaactgcagcagaggcagccgggag

SEQ ID NO: 104 - DNA, 101 bp, Homo sapiens
gcctgctgacagcgcacgatcagttcaaggcaacactgcccgaggctgacygagagcgaggtgccatcatgggcatccagggtgagatccagaagatctgc

SEQ ID NO: 105 - DNA, 101 bp, Homo sapiens
tagcctcaatgacgacctaagctgcacttttccccctagttgtgtcttgcsatgctaaaggacgtcacattgcacaatcttaataaggtttccaatcagcc

SEQ ID NO: 106 - DNA, 101 bp, Homo sapiens
cctagctgcaccccagcgcctgggcccgccccacgctctgtccacacccaygcgccccgggagcggggccatgcctccagccccccagctcgcccgaccca

SEQ ID NO: 107 - DNA, 101 bp, Homo sapiens
aatccccaatgtgagggggtcatgatgatgctgtgggcctctgggcatcartgtcatctcacacccaaagtcagtactcccccaagttctccctatttccc

SEQ ID NO: 108 - DNA, 101 bp, Homo sapiens
cccgtggagggattgtcacttctggttccctgtgggcaggaatggtttccwcgtaggtcactggggttttggccaggaaaagggtatgaaattcatgtgcc

SEQ ID NO: 109 - DNA, 101 bp, Homo sapiens
ctgtcccccacccccaattttctttccaaactcctaagggaggaaagaggrgactcactctttctggcatctgccaccttctcagctgcccgcttctcagc

SEQ ID NO: 110 - DNA, 101 bp, Homo sapiens
tgacaaggagaaatagattagagagaatcacaggagaaatttgagatgcarggccaaaccaaaaagcccaccaaggtcaaaactaaatgaaatgtgaactt

SEQ ID NO: 111 - DNA, 101 bp, Homo sapiens
aaaggctcttacccttggctctccctttcccctcagcctcctgaccaaccyccacatggccctgtgtggcatcccgtgccccctcctcttgggaactgtaa

SEQ ID NO: 112 - DNA, 101 bp, Homo sapiens
aaaaaatttcttgaatggatgagcctgtataccctctacttccaattcacrgtcatcacaacataacagatgaaaaacactcttcatttgtcttaaaagct

SEQ ID NO: 113 - DNA, 101 bp, Homo sapiens
tcccttgggtgtgtaatctaacatagtgacaagttctggagattagggcaygggcatctttgggggttattattctgcttatcccaagaatgttacccttt

SEQ ID NO: 114 - DNA, 101 bp, Homo sapiens
gtgaccttatgttttggcagctttaaaactatgtgatatgcacagtaagtrttttaaaacacattttaattttctccaggactgttagtactaatatgata

SEQ ID NO: 115 - DNA, 101 bp, Homo sapiens
tgagccactgcgcctggccaaaactggtttctagtttatgagttcagcagrtatttgactctggattcctcaatttagtgatatcacacaaaatggtataa

SEQ ID NO: 116 - DNA, 101 bp, Homo sapiens
cctttgagagtttttaatctatcctagacacaggcacagcacgaaaagagraaacatcccagcttcattaggggaaatttatagcttgcctagggtcacca

SEQ ID NO: 117 - DNA, 101 bp, Homo sapiens
gtctaatcctacaatatttccaaacctcacccatcccagaataaataaatrtatgagatgacattgcaaattggacgcccaatgttcacaaaagctgactc

SEQ ID NO: 118 - DNA, 101 bp, Homo sapiens
gacagccaacagcgcgcctaacttggagcgaatcctcttcgggctttccaragtgcgggggatagataaagagtagctggggagacgccccctgaccttgc

SEQ ID NO: 119 - DNA, 101 bp, Homo sapiens
tacttgactttcagtacctcccattgctgagccttttgaggattctcttaygtattcataagtgtgattctcatttttccagtgactcattttccttgtat

SEQ ID NO: 120 - DNA, 101 bp, Homo sapiens
ttctcctctcttaactctcattaggccaactggcaagtttagatgatgtcrtttagaaaaattggtcaaaactagaatataaacataacgtgcaatattcc

SEQ ID NO: 121 - DNA, 101 bp, Homo sapiens
aggagacattcagatcacaaatggttgaaccctgggaggacatcaaaagaytgtttccaaagataagtttctcagaactggaatcctccgaaatgctctgc

SEQ ID NO: 122 - DNA, 101 bp, Homo sapiens
gctcatggagcttcctccagcccagcctctgttcagtttttccaaggcttrtcacagaaagagggctggggtgttatttttaagtcttagctacccagaat

SEQ ID NO: 123 - DNA, 101 bp, Homo sapiens
attgttaaagaagagtgatccctttgtgtttcagcttggcacacagaaackgttttaatttaacagtccagctcctttaatagatcaattctctattgtgg

SEQ ID NO: 124 - DNA, 101 bp, Homo sapiens
gcaaagtctgagtgcttgttaaccagctttgccagttccgtgggtgtggcmtcaggcaattttgcttttagtcgctctgccagtctgaaaaaccatttaaa

SEQ ID NO: 125 - DNA, 101 bp, Homo sapiens
ttccctcttccaagacaatataatatagttatgtcacagttctatttgcayggtgtaaaaaattccatgtttcattgtcttcaacgagtttatgctttgga

SEQ ID NO: 126 - DNA, 101 bp, Homo sapiens
cagagggactactacttgcttccaaagctaacaataaaaaatacctggctktgtgagataattaagagacagagatttgctgggcatggtggctcacgcct

SEQ ID NO: 127 - DNA, 101 bp, Homo sapiens
cctggcagaactaccacctgaacgactggatggaggaggaataccgccacwtcccgggggagtacgtccgcttcaccggctacccctgctcctggaccttc

SEQ ID NO: 128 - DNA, 101 bp, Homo sapiens
gctagtgttttaaagttatgtaaaaagacagactgggcaacatggtgaaayccccatctctacaaaaaagaaaaaaaaaattaacagggtgtggtggtgca

SEQ ID NO: 129 - DNA, 101 bp, Homo sapiens
aaagaaagggggaaagaatgctctgagtgcctactgtattttaagcactgkgacatacacagttttacactgtttaatttaaactttgtagccagtcaatg

SEQ ID NO: 130 - DNA, 101 bp, Homo sapiens
tcaccagtaatggcatggcctggtgtcgggagaacattgacacctcccacrgtgatgtggtgtttgctggcgatggcattgagggctcacctgccaaagat

SEQ ID NO: 131 - DNA, 101 bp, Homo sapiens
gcagcggcacggtcaccttctcactgagctttgatgagcctcagaagaackccatggcccacaggaattctacgcaccagaactccctggaggcccagaaa

SEQ ID NO: 132 - DNA, 101 bp, Homo sapiens
tgttggagcttctgttctcctgcagaaaacctgacaataaacaatgaacabataaataagaacacctccagtagttaagtgctatctgaaaaacaagaagg

SEQ ID NO: 133 - DNA, 101 bp, Homo sapiens
ctgacctgtaatttaatttaaaagccctgagcaggctgggtgcggtacctmattcctataatcccagcattttgggaggctgaggtgggaggattgtttga

SEQ ID NO: 134 - DNA, 101 bp, Homo sapiens
ttctactccttggtcctagctttgttcctaggcgctgtgccgctgtgtcayccaccctgccctgtacaatatgcaggaagcaagcgaggagggggtgcctc

SEQ ID NO: 135 - DNA, 101 bp, Homo sapiens
ttctcaaggacctcctttccctgccctcctgcaccccatcaccccacaagrtttcacagctgcagagaaagcttcatctggtaactagtgttacgggttta

SEQ ID NO: 136 - DNA, 101 bp, Homo sapiens
tttactgttattctggccaagtttgagtggtgatggtgataagtaagtgcrtgtgtgtgtgtgtgtgtgtgtgtgtgtgtgtgtgtgtgtgtgttcaattt

SEQ ID NO: 137 - DNA, 101 bp, Homo sapiens
gaacaaaaaaccaaatgcatacctttgactacaaagttctacttcctctgygtaactcaaaacttaaattccgggagcacaaaagctgcttcagagttgta

SEQ ID NO: 138 - DNA, 101 bp, Homo sapiens
cctgcagcagttcctgtgcctttaaagcctcccctccccccgccccgcccscaggccactaggggagggaaggaggagctgggtcacagcagggaatctta

SEQ ID NO: 139 - DNA, 101 bp, Homo sapiens
atgtcccccacatcccaatactgttttggagaaaggtacttgcatttgcaktatggaaattatttgtattatttcaaacatttggagcatctgcttgcctg

SEQ ID NO: 140 - DNA, 101 bp, Homo sapiens
gttcccccacagggagacccacagcagagacatgactcacaggtggcatcrggtccctttgagtctctctggtgggagaatctcaacccacagagtaggat

SEQ ID NO: 141 - DNA, 101 bp, Homo sapiens
tttttttttccgctgtgctaatgtagggagaagttgttggaggtcacgtcrcagttcacagcaaccatctatgtttgggagcaaggatgctggaaatagaa

SEQ ID NO: 142 - DNA, 101 bp, Homo sapiens
gtttcctcaacagtgaaatagggacatggtcaccttcagggggcagttatraggctcagggtagggtatgttccaggcatggctatgccgcactgtttatc

SEQ ID NO: 143 - DNA, 101 bp, Homo sapiens
agcaatcagaagtgctttccctggatttaataattagatgggagataagayctttgaagtaaagttaaaggcctttctacctagaccacagcattactgaa

SEQ ID NO: 144 - DNA, 101 bp, Homo sapiens
taaacccagcgcagataccgaactggtgtccttcattccagattgcaacamaaccccaaactagcaaacgtttaacaggcgcttggcacccgcaccggtgg

SEQ ID NO: 145 - DNA, 101 bp, Homo sapiens
tctgatgtcaccccctctaattttgagtgatccagtttctttgctttttaygcttgtatctattcttccatcgtagactgacctggtcatttctttggagt

SEQ ID NO: 146 - DNA, 101 bp, Homo sapiens
aagctgcccctcctccaggcccccagtgatgctaagaattcacaccatctsctatccagaaccagtaactgcctgggaggttcctgatgggaatattctgc

SEQ ID NO: 147 - DNA, 101 bp, Homo sapiens
tcacttttgtgattttgtgatgtgtcagtgctgggactgaatccaagttgrgtgacagctggggcgatgcagcagaaggcaggtcttgctttttggtaaca

SEQ ID NO: 148 - DNA, 101 bp, Homo sapiens
aggccactgtatcgcttcgtgtccccgtggaactcataagcagattttgcrctctattaatctacatctgtttgcacgtccctgctgtcagcagcttctgt

SEQ ID NO: 149 - DNA, 101 bp, Homo sapiens
ctgaggtcaagtttttttcatatacctcaaccaaagcaacatactgcaacwgactcaatgcagaggcagataggagaatgcaactatttgattctaagcca

SEQ ID NO: 150 - DNA, 101 bp, Homo sapiens
actctcttatttgtcattggggacggtgtggtatcaacaggtttcacaagktagggggatatgcaccagggctggaacccctctgccttgacggcaccagg

SEQ ID NO: 151 - DNA, 101 bp, Homo sapiens
attttccctcctgctgaactgagctacttctgtgagcattgaaatacttgmaggaacctttgctgcttttctagtcccaggatttgtgagctcactgtctg

SEQ ID NO: 152 - DNA, 101 bp, Homo sapiens
ctgtttcttctggcaaaaaaggttcatcagagtttacaaactccgtgtccygagcttcatcagggtcatcccacacatccccattccaagttgcagggtcc

SEQ ID NO: 153 - DNA, 101 bp, Homo sapiens
cacttcctcatcctaaaatttggctgctaatttctgctgatgctcatggaraatttcccaaagaccctgctcctgaatgaattgaaagcctttgagttgag

SEQ ID NO: 154 - DNA, 101 bp, Homo sapiens
tggattctgtatgtgcgtgtgtgtgcacgtgtgttctgcatgcatctccayggcacattatctggaggtaacatgatcatcaggccttgagctcttttata

SEQ ID NO: 155 - DNA, 101 bp, Homo sapiens
aaatccttaaggaatagagaaggcttgaaatgaagtaggtgcttactaaaygtttgtcaaataaaataaatgagtggatttatgatgctatgcatgaattt

SEQ ID NO: 156 - DNA, 101 bp, Homo sapiens
agctttgttcaatgtattttaatatttattttaatttgcttgcattatctytctttctattaatattcattatttttctttaccttcttttataatgttgg

SEQ ID NO: 157 - DNA, 101 bp, Homo sapiens
attgtctgatgcatcgtgactaagcttggaatgtgccaactgtcccccagsagtggcccttggacagcagagctggagcgccgggactctgagtgcaggaa

SEQ ID NO: 158 - DNA, 101 bp, Homo sapiens
ttcagaactttacagacgtgtcataagtggctcaggagagaggcccactgsacagtggctgcacatggaaggcagagctgaccttgaagagatgaaggaaa

SEQ ID NO: 159 - DNA, 101 bp, Homo sapiens
ctgaagttgatgctgaaaatcaacaagaatatgcaacaaaacccatcaaayactactgaaatagtaaaggccagggtccagcacagtgggtcacacctata

SEQ ID NO: 160 - DNA, 101 bp, Homo sapiens
ttgcaatataatgggtattatatgaaattatcttgggtttgtgcttacatygcaggagtggcaacaaaacaccataatcttttcaatgcttattgcagctg

SEQ ID NO: 161 - DNA, 101 bp, Homo sapiens
gaatataatgttaaacaacaagctcaaacaacaaacaacaaacttcctgtytgcataacttgtattctagtggagaacgtcgaaaataaataaataaataa

SEQ ID NO: 162 - DNA, 101 bp, Homo sapiens
tgttgatgtaccctttagggtgggggaaaggtttggagaactctctggtgygaagagcatgcttgaggtaaccacaagtggtgaacaaagtgcccccaaga

SEQ ID NO: 163 - DNA, 101 bp, Homo sapiens
agagcatagtcctccatgactttcaatgaaaaacccgatagctttcatctyctcaatcctgaagagctgaaggagatttaggctgaacttaaagaaatttt

SEQ ID NO: 164 - DNA, 101 bp, Homo sapiens
cttcatttggaataaatctttgatctggaaccatttccatatttaaaggcyacttcgaatgccatctctgtcatggactttcctctctcctttaagcacaa

SEQ ID NO: 165 - DNA, 101 bp, Homo sapiens
cgtgtttctaaaaatatacgtaataacttgtataatgatgataaagctctrtattacaattgaataagacaggaaaactatttcaagttatttgctgtgtg

SEQ ID NO: 166 - DNA, 101 bp, Homo sapiens
ctaaagaaagaagcaaaaccaggcacagctgatgggttaaccagatatgayacagaaaacatttccttctgctttttggttttaagcctatatttgaagcc

SEQ ID NO: 167 - DNA, 101 bp, Homo sapiens
ctgacattaatatgaatagagcagattcctttgagttaatatttgtctggkgtgttttatttcatccactgacttctaacttttctgtgttcttagagctg

SEQ ID NO: 168 - DNA, 101 bp, Homo sapiens
gtttgtaaaggacctgctcttacatttaaagaaactttttcgcgagggacrgttcaactgaaacttcgaaagcatcattatttgcagagacaggacctgac

SEQ ID NO: 169 - DNA, 101 bp, Homo sapiens
tggaaaattctttagaatagatcatatgttaaatcacaaaacaaaccttarcaaatttgaaaaaaatggaaacatatcaagtattttttaataccacaatg

SEQ ID NO: 170 - DNA, 101 bp, Homo sapiens
ctaactaactaaataaatgataaataaaggcggtgcatgagcactggtgamgggcactttggctgcattgagcacttgcaaatttgaggtgattaaattct

SEQ ID NO: 171 - DNA, 101 bp, Homo sapiens
tcctaggtacatacacgttcacacagctatacacgaagaatctcagccctygtacttttgcatagtctcatacacgtatcagaagcctccacctggctaac

SEQ ID NO: 172 - DNA, 101 bp, Homo sapiens
caaacctccatattcatgtcattgaatgtgggctagtttcagaagggaatktgaaattggacaaggcagctctctttagcagaagcaattctccaacaggg

SEQ ID NO: 173 - DNA, 101 bp, Homo sapiens
gggcttattccaacttggccgtcacagaaagatcctcttcagctttgttgytgaaggatgtttttctgattttagaattctaggtttgtgttaggtgtaga

SEQ ID NO: 174 - DNA, 101 bp, Homo sapiens
atgtaaaaatacacacacacaaagtggagctgagggcaggatggagaactstcattctcagcccatgacctccatggacttggagaaagactcagcctgga

SEQ ID NO: 175 - DNA, 101 bp, Homo sapiens
caacctacaggccacttgtgtcagaatcacgtgaggtacattttaaactgyagatttctgtgcccctcaccagactacagagttggaatctctgagaggtg

SEQ ID NO: 176 - DNA, 101 bp, Homo sapiens
gaatctggctgagcttgggtggcacccaaggatgcctgcagcccgcccagyggcacgggaagccccctcacccgctggctggaaggggtgggaggcaagtg

SEQ ID NO: 177 - DNA, 101 bp, Homo sapiens
tctccctcctcccatctccctcctcctctccatctccctcttctctcatcyctgtctccttcctcctcctgtatcttctccctcctcccatttccctcctc

SEQ ID NO: 178 - DNA, 101 bp, Homo sapiens
acacatgcaggtttgttacataggtaaactcgtgtcatgggggttcagtgyacaggttatttcatcacctagctactaaatgtagtacctgatagttattt

SEQ ID NO: 179 - DNA, 101 bp, Homo sapiens
tccagtgcctgctgacaagaacgaaggcccgggcgattattctcaatagaytggctttcttctgctgttgctgctgttgtgtgtacatagattttgtcccc

SEQ ID NO: 180 - DNA, 101 bp, Homo sapiens
atcaaagaagtggctgcttcatcacattcacaggccttgtccacagccaaraggaagagatcagaggttctgttcaccggcggggtaggactcttggaatt

SEQ ID NO: 181 - DNA, 101 bp, Homo sapiens
aaggaaaagttcaatatagagattatcattgcctgggagacatttgcttcrccttctgttttcaaatgcttgcaacatagaagactggagtgaatccaaga

SEQ ID NO: 182 - DNA, 101 bp, Homo sapiens
aaagtcaccctttgcttcatcccttctccctttattgtgcacctattatadgttatacaatgtgtttgagcagatgcaaaaaataagactaatttactaac

SEQ ID NO: 183 - DNA, 101 bp, Homo sapiens
atccctggacaagatcgtaagtaaaaataacatttcatcctaggaggcacrgagagattagagccacccttaaggatacaatggatatggagctggtggtc

SEQ ID NO: 184 - DNA, 101 bp, Homo sapiens
ttccatgtttcataagttcttaagtgattcttctttaatcccactaaatcrtgactccagatgagtttaagaaatcttaagactattttttaattattaca

SEQ ID NO: 185 - DNA, 101 bp, Homo sapiens
taagagggaaagctagcaatcccacaatgatttcctttatttcttgccatmcgaatatatccttcttcaccaaagttgtggccccagctttagaaaaagaa

SEQ ID NO: 186 - DNA, 101 bp, Homo sapiens
cctgatacgacctgtccttctcaaaacacctaaacttgggagaacattgtyccccagtgctggggtaggagagtctgcctgttattctgcctctatgcaga

SEQ ID NO: 187 - DNA, 100 bp, Homo sapiens
agttctcacagggcatgggctaacaattatggtactgctctacatgtacwctggaaggggacaagcaagtaaatggatggtggatggtgggacccagatt

SEQ ID NO: 188 - DNA, 101 bp, Homo sapiens
aattatgtaccaatattggttcattggttatgaccaatgcacctcacttayataggatagaaataataggggagattgggtgtgaagtatatgggaattct

SEQ ID NO: 189 - DNA, 101 bp, Homo sapiens
gggagttgtagttttattacataaaattgccagccgaggatagggaaaacrgtatttactagcctcggggaacctcggaatctgcatctcagccttctcca

SEQ ID NO: 190 - DNA, 101 bp, Homo sapiens
gctttacattcctccatgacactaatcaccatctgatgtactgttttcctsatctgtttattgtcatttttccccactagacttcaagttccatgaaagag

SEQ ID NO: 191 - DNA, 101 bp, Homo sapiens
caacatgtcatgtgcaatgaaaccagataacagaagaaagggaaactctcrttttttgtttagatgttattaatgtgtcacacatttatacacatggcact

SEQ ID NO: 192 - DNA, 101 bp, Homo sapiens
gctgggattacgggcatgagccactgtgccaggcctcttttttacacagarttgtttttgtggaaatacgattgtcaggttaacaatgactactgttattc

SEQ ID NO: 193 - DNA, 101 bp, Homo sapiens
acattgcatttgcctccaaagctcaaaaacagaatgaagcatcacatcaaygtcagcttctctttttaaagaaaaattttctctcaaaagtgtcccaatat

SEQ ID NO: 194 - DNA, 101 bp, Homo sapiens
agatggcagttgcagtgagctataatcaagcaactgcactgcaatccagcytgggctggtgagggagactctgtaaaaaaaaaaaatcagctcctcagtgg

SEQ ID NO: 195 - DNA, 101 bp, Homo sapiens
tatgactaccctgtgtgattcaataaattttccaggactctggtatgacayactgtttgcattcgactgtttcctttccctcttaagcatttggcccccag

SEQ ID NO: 196 - DNA, 101 bp, Homo sapiens
aactagattgaaccctcagcctagcaatgtcactatgctacacttttcctrgtgtggtctacccgagatgaggggctgaggtttttttttgtttttgtttc

SEQ ID NO: 197 - DNA, 101 bp, Homo sapiens
tagaaaccagctccttggacagctcaccaaaaggctaggatgttgaagacrtgctccactcctctcatttcctcctgaggctgaatcctcgggttgcgtac

SEQ ID NO: 198 - DNA, 101 bp, Homo sapiens
aacaactatttcccagtttttgtaaaattgttcatttcttagctcctcctyagcctttatttaatccatacactcttaaatctttgcttggatcaataaga

SEQ ID NO: 199 - DNA, 101 bp, Homo sapiens
gtctcttttctggtgattaaaagtcattatcacctagtcattactaccaaygagataattaagacatttcaaacaaacaatttaaaacaagatgtattcct

SEQ ID NO: 200 - DNA, 101 bp, Homo sapiens
acagatgtgcatgcagacatgagcacacgcacaccagaacactagagtcgrccgcatcctcctcacttggctgatgcccccttctgcttgatttcatcaca

SEQ ID NO: 201 - DNA, 101 bp, Homo sapiens
cattcctttttttcacctaaatagcattctctgtcttggccaagctgaccygtccctgtcttccacatgcatcttgcactttttgatgtcctgttattcac

SEQ ID NO: 202 - DNA, 101 bp, Homo sapiens
aatgtcttttcacatatttgaaattacctgaacctatcaccaaggtcataygcatcatccatgtatgactttgccccacttgccaaatggggcagaccaag

SEQ ID NO: 203 - DNA, 101 bp, Homo sapiens
agttttctttcttttcttttcttttcttttttttgtttgtttgtcatgtcrttccctggttttggtactggctttatagaatgatggtgatactggcttta

SEQ ID NO: 204 - DNA, 101 bp, Homo sapiens
gtgtcccaggcactgtgttaggattcactgagttgtcacaaaaatcctgcraagttcattctcgaggacgctctggaatttaaataccttgccaagatcat

SEQ ID NO: 205 - DNA, 101 bp, Homo sapiens
aagcgattctcctgccttagcctttctagtagctgggattacaggcatgcrccaccatgcccagctaattttttatttttagtagagacggggtttctcca

SEQ ID NO: 206 - DNA, 101 bp, Homo sapiens
cagctcattcatgaacaatgactgagtatatgtgatccaaatgcacagggkgttatcctgagaaagcaatcagccttgtgggccagataaatccattataa

SEQ ID NO: 207 - DNA, 101 bp, Homo sapiens
attctccagggatttgctaccatctttattgtctgaaaaagaatttgatabgtcatattctctattctgttcatattttaatatctgaagcctatgctcat

SEQ ID NO: 208 - DNA, 101 bp, Homo sapiens
aactggaactaggttaagcaaagtaacatttcaaaagggaagattcagtgkaagttttctgggattgctcacagaatccaagaatgggctgcaggtatcag

SEQ ID NO: 209 - DNA, 101 bp, Homo sapiens
tatcccatccctgtattatcagtatacgttggacatatatgaggcaaatayctttttcatattgagaggtcttcatattgagagaaattgtataagacaac

SEQ ID NO: 210 - DNA, 101 bp, Homo sapiens
gaacagttaaagatgacttacttttttttttttttcatttataaaaatgcyatggacccttttaagagaatcggcatcatgaaatgagagagaaagtagga

SEQ ID NO: 211 - DNA, 101 bp, Homo sapiens
cagcagttttggagatggaagcaatgccagagatgggactatttcttcttmttgttttagatgtaaacattaaaaaaaaaaaaacaggatgcacacttagt

SEQ ID NO: 212 - DNA, 101 bp, Homo sapiens
gccaaagatcctgaataccaagccctcagaaaatggtaaagcactgtaagyatcttaattgtcagcattatcacaactacaaatggcaaagctgggtggag

SEQ ID NO: 213 - DNA, 101 bp, Homo sapiens
ttttataatttaggaatttccacattattaagtcaggatagccagtatagyagagatacaggtgtccaatatccctttcatccttcttcctttagtaatag

SEQ ID NO: 214 - DNA, 101 bp, Homo sapiens
gggaaaaaattaattgttgttaagaattatggtgattctgctccatagcarcttcattaaaggacctagtctaagttcaagattaaaaggttatatgaggc

SEQ ID NO: 215 - DNA, 101 bp, Homo sapiens
ttaaatataagcccataggcactctgctgcatgcagattctatctcaaaayaaaacactctgaagatgttccaagacccacacatacagattcttttcctt

SEQ ID NO: 216 - DNA, 101 bp, Homo sapiens
tgaccttctgaacacgattgatgacagcacaggtgatgcaactctgacatytgtggtggccccagctgcttttgtacctgcgaggagggcaggctgtgcag

SEQ ID NO: 217 - DNA, 101 bp, Homo sapiens
tgagccagtctcttctctttaagttgatgctagctgccgttttgtgttatytgttacagactaatacaatttgcaataattgaagatgcaatatttattga

SEQ ID NO: 218 - DNA, 101 bp, Homo sapiens
tgcctcctcctcctcctcctcgttaaactggttaattaatggctgctgccygtgggaagcagatgttctggagctgttggcctggggaggcattggtctgg

(19) United States
(12) Patent Application Publication
(10) Pub. No.: US 2021/0287758 A1
(43) Pub. Date: Sep. 16, 2021

(54) ANCESTRY-SPECIFIC GENETIC RISK SCORES

(71) Applicant: Asia Genomics Pte. Ltd., Singapore (SG)

(72) Inventors: Mun Yew WONG, Singapore (SG); Jia Yi HAR, Singapore (SG); Pauline C. NG, Singapore (SG); Chun Meng ONG, Singapore (SG); Robert Keams VALENZUELA, Marshfield, WI (US); Vishweshwaran SRIDHAR, Singapore (SG)

(21) Appl. No.: 17/332,902

(22) Filed: May 27, 2021

Related U.S. Application Data
(63) Continuation of application No. PCT/IB2019/001179, filed on Oct. 31, 2019.
Application No. PCT/IB2019/001179 is a continuation-in-part of application No. 16/216,940, filed on Dec. 11, 2018, now Pat. No. 10,468,141.
(60) Provisional application No. 62/772,565, filed on Nov. 28, 2018.
(60) Provisional application No. 62/772,565, filed on Nov. 28, 2018.

(51) Int. Cl. G16B 20/40; C12Q 1/6827; G16B 10/00; G16B 20/20; G16B 40/20; C12Q 1/6883; G16H 10/40; G16H 50/30

(52) U.S. Cl. CPC G16B 20/40; C12Q 1/6827; G16B 10/00; G16B 20/20; C12Q 2600/158; C12Q 1/6883; G16H 10/40; G16H 50/30; C12Q 2600/156; G16B 40/20

(57) ABSTRACT

Disclosed herein are methods and systems for calculating genetic risk scores (GRS) representing the likelihood that an individual will develop a specific trait based on the ancestry of the individual. Also provided are methods and systems for providing a recommendation to the individual to modify a behavior related to a specific trait, based on the individual's GRS for that trait.

### CROSS-REFERENCE

This application is a continuation of PCT/IB2019/001179, filed Oct. 31, 2019, which claims the benefit of both U.S. Provisional Application No. 62/772,565, filed Nov. 28, 2018, and U.S. patent application Ser. No. 16/216,940, filed Dec. 11, 2018, which claims the benefit of U.S. Provisional Application No. 62/772,565, each of which is hereby incorporated by reference herein in its entirety.

### SEQUENCE LISTING

The instant application contains a Sequence Listing which is hereby incorporated by reference in its entirety. Said ASCII copy, created on Oct. 28, 2019, is named 55075-701_301_SL.txt and is 47.9 KB in size.

### SUMMARY OF THE INVENTION

Genome Wide Association Studies (GWAS) have enabled scientists to identify genetic variations that are associated with a wide range of phenotypic traits. A genetic risk score (GRS) is used to predict whether an individual will develop a trait based on a presence of certain genetic variants detected in a sample obtained from that individual. However, data show that genetic variation and patterns underlying discrete ancestral populations differ. Thus, whether the detected genetic variants confer a risk that the individual will develop the trait depends in large part on the ancestry of that individual. Current genetic risk prediction methods either do not account for the ancestry of the individual at all, or account for ancestry using consumer surveys leading to imprecise, and often, inaccurate genetic risk predictions.

Disclosed herein, in certain embodiments, are methods, media, and systems for calculating a GRS by analyzing the genotype of the individual to determine an ancestry of the individual and calculating a GRS based on the ancestry-specific genetic risk variants derived from GWAS of subjects of the same ancestry as the individual. In some embodiments, genetic variant(s) accounted for in a GRS may include single nucleotide variants (SNVs), insertions or deletions of nucleotide bases (indels), or copy number variants (CNVs). In some embodiments, if a genetic variant detected in a sample obtained from the individual does not correspond to genetic variant reported in the GWAS of the ancestry-specific subject group (unknown genetic variant), a proxy genetic variant is selected based on the non-random association, known as linkage disequilibrium (LD), with the unknown genetic variant within the particular ancestral population, which serves as the basis for risk prediction. Studies show that patterns in LD in the human genome differ across different ancestral populations.

Disclosed herein, in some embodiments, are computer-implemented methods comprising: (a) assigning an ancestry of an individual using a distance-based or a models-based computer program to analyze a genotype of the individual, the genotype comprising one or more individual-specific genetic variants; and (b) detecting in the genotype of the individual, an ancestry-specific variant associated with a specific phenotypic trait, the ancestry-specific variant corresponding to: (i) an individual-specific genetic variant detectable in the genotype of the individual; or (ii) a genetic variant in linkage disequilibrium (LD) with the individual-specific genetic variant as determined by imputing the individual-specific variant missing from ancestry-specific phased haplotypes determined using a reference group of individuals that has the same ancestry as the individual; and (c) calculating a genetic risk score (GRS) for the individual based on the ancestry-specific variant detected in (b), wherein the GRS is indicative of a likelihood that the individual has, or will develop, the specific phenotypic trait. In some embodiments the ancestry-specific genetic variant and the individual-specific genetic variant is selected from the group consisting of a single nucleotide variant (SNV), a copy number variant (CNV), and an indel. In some embodiments imputing in step (ii) comprises: (1) phasing unphased genotype data from the individual to generate ancestry-specific phased haplotypes based on the ancestry of the individual; and (2) imputing individual-specific genotypes not present in the ancestry-specific phased haplotypes using phased haplotype data from the reference group that has the same ancestry as the individual to select the genetic variant in LD with the individual-specific genetic variant. In some embodiments the LD is defined by a D′ value comprising at least about 0.80 or a r2 value comprising at least 0.80. In some embodiments the specific trait comprises a nutritional trait, a clinical trait, a subclinical trait, a physical exercise trait, a skin trait, an allergy trait, or a mental trait, or combination thereof. In some embodiments the genotype of the individual is obtained by subjecting, or having subjected, genetic material obtained from the individual to a genotyping assay. In some embodiments the genotyping assay comprises a deoxyribonucleic acid (DNA) array, ribonucleic acid (RNA) array, sequencing assay, or a combination thereof. In some embodiments, the distance-based computer program is principle component analysis, and wherein the models-based computer program is a maximum likelihood or a Bayesian method. In some embodiments the GRS for the individual based on the ancestry-specific variant is more accurate than a corresponding GRS of the individual based on a variant that is not ancestry-specific. In some embodiments, the computer-implemented methods further comprise providing a notification comprising the GRS for the specific phenotypic trait of the individual. In some embodiments the notification further comprises a behavior recommendation to the individual based on the GRS for the specific phenotypic trait. In some embodiments the behavioral modification related to the specific phenotypic trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption. In some embodiments, the subclinical trait comprises a phenotype of a disease or condition. In some embodiments, the physical exercise trait comprises exercise aversion, aerobic performance, difficulty losing weight, endurance, power, fitness benefits, reduced heart beat response to exercise, lean body mass, muscle soreness, muscle damage risk, muscle repair impairment, stress fracture, overall injury risk, potential for obesity, or resting metabolic rate impairment. In some embodiments, the skin trait comprises collagen breakdown, dryness, antioxidant deficiency, detoxification impairment, skin glycation, pigmented spots, youthfulness, photoaging, dermal sensitivity, or sensitivity to sun. In some embodiments, the hair trait comprises hair thickness, hair thinning, hair loss, baldness, oiliness, dryness, dandruff, or hair volume. In some embodiments, the nutritional trait comprises vitamin deficiency, mineral deficiency, antioxidant deficiency, fatty acid deficiency, metabolic imbalance, metabolic impairment, metabolic sensitivity, allergy, satiety, or the effectiveness of a healthy diet. In some embodiments, the vitamin deficiency comprises a deficiency of a vitamin comprising Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, and Vitamin K. In some embodiments, the mineral deficiency comprises a deficiency of a mineral comprising calcium, iron, magnesium, zinc, or selenium. In some embodiments, the antioxidant deficiency comprises a deficiency of an antioxidant comprising glutathione, or coenzyme Q10 (CoQ10). In some embodiments, the fatty acid deficiency comprises a deficiency in polyunsaturated fatty acids or monounsaturated fatty acids. In some embodiments, the metabolic imbalance comprises glucose imbalance. In some embodiments, the metabolic impairment comprises impaired metabolism of caffeine or drug therapy. In some embodiments, the metabolic sensitivity comprises gluten sensitivity, glycan sensitivity, or lactose sensitivity. In some embodiments, the allergy comprises an allergy to food (food allergy) or environmental factors (environmental allergy). In some embodiments, the methods further comprise administering a treatment to the individual effective to ameliorate or prevent the specific trait in the individual, provided the genetic risk score indicates a high likelihood that the individual has, or will develop, the specific trait. In some embodiments, the treatment comprises a supplement or drug therapy. In some embodiments, the supplement comprises a vitamin, mineral, probiotic, anti-oxidant, anti-inflammatory, or combination thereof. In some embodiments, the behavioral modification related to the specific trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption.

Disclosed herein, in some embodiments, are systems comprising: a computing device comprising at least one processor, a memory, and a software program including instructions executable by at least one processor to assess a likelihood that an individual has, or will develop, a specific phenotypic trait, the instructions comprising the steps of: (a) assigning an ancestry of an individual using a distance-based or a models-based computer program to analyze a genotype of the individual, the genotype comprising one or more individual-specific genetic variants; and (b) detecting in the genotype of the individual, an ancestry-specific variant associated with a specific phenotypic trait, the ancestry-specific variant corresponding to: (i) an individual-specific genetic variant detectable in the genotype of the individual; or (ii) a genetic variant in linkage disequilibrium (LD) with the individual-specific genetic variant as determined by imputing the individual-specific variant missing from ancestry-specific phased haplotypes determined using a reference group of individuals that has the same ancestry as the individual; and (c) calculating a genetic risk score (GRS) for the individual based on the ancestry-specific variant detected in (b), wherein the GRS is indicative of a likelihood that the individual has, or will develop, the specific phenotypic trait. In some embodiments the ancestry-specific genetic variant and the individual specific genetic variant is selected from the group consisting of a single nucleotide variant (SNV), a copy number variant (CNV), and an indel. In some embodiments, the imputing in step (2) comprises: (1) phasing unphased genotype data from the individual to generate ancestry-specific phased haplotypes based on the ancestry of the individual; and (2) imputing individual-specific genotypes not present in the ancestry-specific phased haplotypes using phased haplotype data from the reference group that has the same ancestry as the individual to select the genetic variant in LD with the individual-specific genetic variant. In some embodiments the LD is defined by a D′ value comprising at least about 0.80 or a r2 value comprising at least 0.80. In some embodiments, the specific trait comprises a nutritional trait, a clinical trait, a subclinical trait, a physical exercise trait, a skin trait, an allergy trait, or a mental trait. In some embodiments, the systems further comprise a genotyping assay. In some embodiments, the genotyping assay comprises a deoxyribonucleic acid (DNA) array, ribonucleic acid (RNA) array, sequencing assay, or a combination thereof. In some embodiments, the distance-based computer program is principle component analysis, and wherein the models-based computer program is a maximum likelihood or a Bayesian method. In some embodiments, the GRS for the individual based on the ancestry-specific variant is more accurate than a corresponding GRS of the individual based on a variant that is not ancestry-specific. In some embodiments, the systems further comprise a reporting module configured to generate a report comprising the GRS of the individual for the specific phenotypic trait. In some embodiments, the systems further comprise an output module configured to display the report to the individual. In some embodiments, the report comprises the risk that the individual has, or will develop, the specific trait. In some embodiments, the report further comprises a recommendation of a behavior recommendation to the individual based on the GRS for the specific phenotypic trait. In some embodiments, the behavioral modification related to the specific phenotypic trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption. In some embodiments, the subclinical trait comprises a phenotype of a disease or condition. In some embodiments, the physical exercise trait comprises exercise aversion, aerobic performance, difficulty losing weight, endurance, power, fitness benefits, reduced heart beat response to exercise, lean body mass, muscle soreness, muscle damage risk, muscle repair impairment, stress fracture, overall injury risk, potential for obesity, or resting metabolic rate impairment. In some embodiments, the skin trait comprises collagen breakdown, dryness, antioxidant deficiency, detoxification impairment, skin glycation, pigmented spots, youthfulness, photoaging, dermal sensitivity, or sensitivity to sun. In some embodiments, the hair trait comprises hair thickness, hair thinning, hair loss, baldness, oiliness, dryness, dandruff, or hair volume. In some embodiments, the nutritional trait comprises vitamin deficiency, mineral deficiency, antioxidant deficiency, fatty acid deficiency, metabolic imbalance, metabolic impairment, metabolic sensitivity, allergy, satiety, or the effectiveness of a healthy diet. In some embodiments, the vitamin deficiency comprises a deficiency of a vitamin comprising Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, and Vitamin K. In some embodiments, the mineral deficiency comprises a deficiency of a mineral comprising calcium, iron, magnesium, zinc, or selenium. In some embodiments, the antioxidant deficiency comprises a deficiency of an antioxidant comprising glutathione, or coenzyme Q10 (CoQ10). In some embodiments, the fatty acid deficiency comprises a deficiency in polyunsaturated fatty acids or monounsaturated fatty acids. In some embodiments, the metabolic imbalance comprises glucose imbalance. In some embodiments, the metabolic impairment comprises impaired metabolism of caffeine or drug therapy. In some embodiments, the metabolic sensitivity comprises gluten sensitivity, glycan sensitivity, or lactose sensitivity. In some embodiments, the allergy comprises an allergy to food (food allergy) or environmental factors (environmental allergy). In some embodiments, the systems further comprise administering a treatment to the individual effective to ameliorate or prevent the specific trait in the individual, provided the genetic risk score indicates a high likelihood that the individual has, or will develop, the specific trait. In some embodiments, the treatment comprises a supplement or drug therapy. In some embodiments, the supplement comprises a vitamin, mineral, probiotic, anti-oxidant, anti-inflammatory, or combination thereof. In some embodiments, the behavioral modification related to the specific trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption.

Disclosed herein, in some embodiments, are uses of the system of the present disclosure for recommending a behavior modification or a product to the individual, based on the GRS calculated in (c).

Also disclosed herein, in some embodiments, are non-transitory computer readable storage media, comprising computer-executable code configured to cause at least one processor to perform steps in the methods disclosed herein.

Disclosed herein, in certain embodiments, are computer-implemented methods for recommending a behavioral modification to an individual based on an ancestry and a genotype of the individual, the method comprising: a) providing the genotype of the individual, the genotype comprising one or more individual-specific genetic variants; b) assigning an ancestry to the individual based, at least in part, on the genotype of the individual; c) using a trait-associated variants database comprising ancestry-specific genetic variants derived from subjects with the same ancestry as the individual (subject group) to select one or more ancestry-specific genetic variants based, at least in part, on the ancestry of the individual, wherein each of the one or more ancestry-specific genetic variants correspond to: (i) an individual-specific genetic variant of the one or more individual-specific genetic variants, or (ii) a predetermined genetic variant in linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk; (d) calculating a genetic risk score for the individual based on the selected one or more ancestry-specific genetic variants, wherein the genetic risk score is indicative of the likelihood that the individual has, or will develop the specific trait; and (e) providing a recommendation to the individual comprising a behavioral modification related to the specific trait based on the genetic risk score. In some embodiments, the methods further comprise providing a survey to the individual comprising one or more questions relating to the specific trait. In some embodiments, the methods further comprise receiving, from the individual, one or more answers to one or more questions relating to the specific trait in a survey provided to the individual. In some embodiments, the methods further comprise: a) providing a survey to the individual comprising one or more questions relating to the specific trait; and b) receiving, from the individual, one or more answers to the one or more questions, wherein the recommendation to the individual comprising the behavioral modification related to the specific trait is further based on the one or more answers provided by the individual. In some embodiments, the methods further comprise storing, in a trait-associated variants database, the ancestry-specific genetic variants associated with the specific trait derived from the subject group. In some embodiments, the genetic risk score comprises a percentile or z-score. In some embodiments, the LD is defined by (i) D′ value of at least about 0.20, or (ii) an r² value of at least about 0.70. In some embodiments, the LD is defined by a D′ value comprising between about 0.20 and 0.25, 0.25 and 0.30, 0.30 and 0.35, 0.35 and 0.40, 0.40 and 0.45, 0.45 and 0.50, 0.50 and 0.55, 0.55 and 0.60, 0.60 and 0.65, 0.65 and 0.70, 0.70 and 0.75, 0.75 and 0.80, 0.80 and 0.85, 0.85 and 0.90, 0.90 and 0.95, or 0.95 and 1.0. In some embodiments, the LD is defined by a D′ value comprising at least about 0.20, 0.25, 0.30, 0.35, 0.40, 0.45, 0.50, 0.55, 0.60, 0.65, 0.70, 0.75, 0.80, 0.85, 0.85, 0.90, 0.95 and 1.0. In some embodiments, the LD is defined by a r² value comprising at least about 0.70, 0.75, 0.80, 0.85, 0.90, 0.95 and 1.0. In some embodiments, the LD is defined by a r² value comprising between about 0.70 and 0.75, 0.75 and 0.80, 0.80 and 0.85, 0.85 and 0.90, 0.90 and 0.95, or 0.95 and 1.0. In some embodiments, the genotype of the individual is obtained by subjecting, or having subjected, genetic material obtained from the individual to a genotyping assay. In some embodiments, genotype of the individual is obtained by subjecting the genetic material obtained from the individual to a deoxyribonucleic acid (DNA) array, ribonucleic acid (RNA) array, sequencing assay, or a combination thereof. In some embodiments, the sequencing assay comprises next generation sequencing (NGS). In some embodiments, the methods further comprise updating the trait-associated variants database with the assigned ancestry, a specific trait, and the genotype of the individual. In some embodiments, ancestry is assigned to the individual in (b) using a principle component analysis (PCA), or a maximum likelihood estimation (MLE), or a combination thereof. In some embodiments, the one or more ancestry-specific genetic variants, the one or more individual-specific genetic variants, and the genetic variants in LD with the one or more individual-specific genetic variants comprise a Single Nucleotide Variant (SNV). In some embodiments, the one or more units of risk comprises a risk allele. In some embodiments, the one or more ancestry-specific genetic variants, the one or more individual-specific genetic variants, and the genetic variants in LD with the one or more individual-specific genetic variants comprise an indel characterized by an insertion or a deletion of one or more nucleotides. In some embodiments, the one or more units of risk comprises an insertion (I) or deletion (D) of a nucleotide base. In some embodiments, the one or more ancestry-specific genetic variants, or the one or more individual-specific genetic variants comprise a Copy Number Variant (CNV). In some embodiments, the one or more units of risk comprises a duplication or a deletion of a nucleic acid sequence. In some embodiments, the nucleic acid sequence comprises about two, three, four, five, six, seven, eight, nine, or ten, nucleotides. In some embodiments, the nucleic acid sequence comprises more than three nucleotides. In some embodiments, the nucleic acid sequence comprises an entire gene. In some embodiments, the methods further comprise providing a notification to the individual of the risk that the individual has, or will develop, the specific trait. In some embodiments, the specific trait comprises a nutritional trait, a clinical trait, a subclinical trait, a physical exercise trait, a skin trait, a hair trait, an allergy trait, or a mental trait. In some embodiments, the clinical trait comprises a disease or condition. In some embodiments, the subclinical trait comprises a phenotype of a disease or condition. In some embodiments, the physical exercise trait comprises exercise aversion, aerobic performance, difficulty losing weight, endurance, power, fitness benefits, reduced heart beat response to exercise, lean body mass, muscle soreness, muscle damage risk, muscle repair impairment, stress fracture, overall injury risk, potential for obesity, or resting metabolic rate impairment. In some embodiments, the skin trait comprises collagen breakdown, dryness, antioxidant deficiency, detoxification impairment, skin glycation, pigmented spots, youthfulness, photoaging, dermal sensitivity, or sensitivity to sun. In some embodiments, the hair trait comprises hair thickness, hair thinning, hair loss, baldness, oiliness, dryness, dandruff, or hair volume. In some embodiments, the nutritional trait comprises vitamin deficiency, mineral deficiency, antioxidant deficiency, fatty acid deficiency, metabolic imbalance, metabolic impairment, metabolic sensitivity, allergy, satiety, or the effectiveness of a healthy diet. In some embodiments, the vitamin deficiency comprises a deficiency of a vitamin comprising Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, and Vitamin K. In some embodiments, the mineral deficiency comprises a deficiency of a mineral comprising calcium, iron, magnesium, zinc, or selenium. In some embodiments, the antioxidant deficiency comprises a deficiency of an antioxidant comprising glutathione, or coenzyme Q10 (CoQ10). In some embodiments, the fatty acid deficiency comprises a deficiency in polyunsaturated fatty acids or monounsaturated fatty acids. In some embodiments, the metabolic imbalance comprises glucose imbalance. In some embodiments, the metabolic impairment comprises impaired metabolism of caffeine or drug therapy. In some embodiments, the metabolic sensitivity comprises gluten sensitivity, glycan sensitivity, or lactose sensitivity. In some embodiments, the allergy comprises an allergy to food (food allergy) or environmental factors (environmental allergy). In some embodiments, the methods further comprise administering a treatment to the individual effective to ameliorate or prevent the specific trait in the individual, provided the genetic risk score indicates a high likelihood that the individual has, or will develop, the specific trait. In some embodiments, the treatment comprises a supplement or drug therapy. In some embodiments, the supplement comprises a vitamin, mineral, probiotic, anti-oxidant, anti-inflammatory, or combination thereof. In some embodiments, the behavioral modification related to the specific trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption. In some embodiments, the recommendation is displayed in a report. In some embodiments, the report is displayed to the individual via a user interface of an electronic device. In some embodiments, the report further comprises the genetic risk score for the individual for the specific trait. In some embodiments, the genetic risk score is calculated by: a) calculating a raw score comprising a total number of the one or more units of risk for each ancestry-specific genetic variant for each subject of the subject group, thereby generating an ancestry-specific observed range of raw scores; b) calculating a total number of the one or more units of risk for each of the one or more individual-specific genetic variants, thereby generating an individual raw score; and c) comparing the individual raw score with the ancestry-specific observed range to generate the genetic risk score. In some embodiments, the genetic risk score is calculated by: a) determining an odds ratio for each of the ancestry-specific genetic risk variants; and b) if two or more ancestry-specific genetic variants are selected, then multiplying the odds ratio for each of the two or more ancestry-specific genetic variants together. In some embodiments, the genetic risk score is calculated by: a) determining a relative risk for each of the ancestry-specific genetic risk variants; and b) if two or more ancestry-specific genetic variants are selected, then multiplying the relative risks for each of the two or more ancestry-specific genetic variants together. In some embodiments, the predetermined genetic variant is determined by a) providing unphased genotype data from an individual; b) phasing the unphased genotype data to generate individual-specific phased haplotypes based on the ancestry of the individual; c) imputing individual-specific genotypes not present in the phased individual-specific phased haplotypes using phased haplotype data from a reference group that has the same ancestry as the individual; and d) selecting a genetic variant from the imputed individual-specific genotypes that is in linkage disequilibrium (LD) an individual-specific genetic variant associated with a likelihood that the individual has, or will develop, a specific trait.

Disclosed herein, in certain embodiments, are computer-implemented methods of determining a likelihood that an individual has, or will develop, a specific trait based on the ancestry of the individual, the method comprising: a) providing the genotype of the individual, the genotype comprising one or more individual-specific genetic variants; b) assigning an ancestry to the individual based, at least in part, on the genotype of the individual; c) using a trait-associated variants database comprising ancestry-specific genetic variants derived from subjects with the same ancestry as the individual (subject group) to select one or more ancestry-specific genetic variants based, at least in part, on the ancestry of the individual, wherein each of the one or more ancestry-specific genetic variants correspond to: (i) an individual-specific genetic variant of the one or more individual-specific genetic variants, or (ii) a predetermined genetic variant in a linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk; and (d) calculating a genetic risk score for the individual based on the selected one or more ancestry-specific genetic variants, wherein the genetic risk score is indicative of the likelihood that the individual has, or will develop the specific trait. In some embodiments, the methods further comprise providing a notification to the individual of the risk that the individual has, or will develop, the specific trait. In some embodiments, the notification comprises a recommendation for a behavior modification related to the specific trait. In some embodiments, the behavioral modification related to the specific trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption. In some embodiments, the notification is displayed in a report. In some embodiments, the report is displayed to the individual via a user interface of an electronic device. In some embodiments, the methods further comprise providing a survey to the individual comprising one or more questions relating to the specific trait. In some embodiments, the methods further comprise receiving, from the individual, one or more answers to one or more questions relating to the specific trait in a survey provided to the individual. In some embodiments, the methods further comprise: a) providing a survey to the individual comprising one or more questions relating to the specific trait; and b) receiving, from the individual, one or more answers to the one or more questions, wherein the recommendation to the individual comprising the behavioral modification related to the specific trait is further based on the one or more answers provided by the individual. In some embodiments, the methods further comprise storing, in a trait-associated variants database, the ancestry-specific genetic variants associated with the specific trait derived from the subject group. In some embodiments, the genetic risk score comprises a percentile or z-score. In some embodiments, the LD is defined by (i) D′ value of at least about 0.20, or (ii) an r² value of at least about 0.70. In some embodiments, the LD is defined by a D′ value comprising between about 0.20 and 0.25, 0.25 and 0.30, 0.30 and 0.35, 0.35 and 0.40, 0.40 and 0.45, 0.45 and 0.50, 0.50 and 0.55, 0.55 and 0.60, 0.60 and 0.65, 0.65 and 0.70, 0.70 and 0.75, 0.75 and 0.80, 0.80 and 0.85, 0.85 and 0.90, 0.90 and 0.95, or 0.95 and 1.0. In some embodiments, the LD is defined by a r² value comprising between about 0.70 and 0.75, 0.75 and 0.80, 0.80 and 0.85, 0.85 and 0.90, 0.90 and 0.95, or 0.95 and 1.0. In some embodiments, the LD is defined by a D′ value comprising at least about 0.20, 0.25, 0.30, 0.35, 0.40, 0.45, 0.50, 0.55, 0.60, 0.65, 0.70, 0.75, 0.80, 0.85, 0.85, 0.90, 0.95 and 1.0. In some embodiments, the LD is defined by a r² value comprising at least about 0.70, 0.75, 0.80, 0.85, 0.90, 0.95 and 1.0. In some embodiments, the genotype of the individual is obtained by subjecting, or having subjected, genetic material obtained from the individual to a genotyping assay. In some embodiments, genotype of the individual is obtained by subjecting the genetic material obtained from the individual to a deoxyribonucleic acid (DNA) array, ribonucleic acid (RNA) array, sequencing assay, or a combination thereof. In some embodiments, the sequencing assay comprises next generation sequencing (NGS). In some embodiments, the methods further comprise updating the trait-associated variants database with the assigned ancestry, a specific trait, and the genotype of the individual. In some embodiments, ancestry is assigned to the individual in (b) using a principle component analysis (PCA), or a maximum likelihood estimation (MLE), or a combination thereof. In some embodiments, the one or more ancestry-specific genetic variants, the one or more individual-specific genetic variants, and the genetic variants in LD with the one or more individual-specific genetic variants comprise a Single Nucleotide Variant (SNV). In some embodiments, the one or more units of risk comprises a risk allele. In some embodiments, the one or more ancestry-specific genetic variants, the one or more individual-specific genetic variants, and the genetic variants in LD with the one or more individual-specific genetic variants comprise an indel characterized by an insertion or a deletion of one or more nucleotides. In some embodiments, the one or more units of risk comprises a insertion (I) or a deletion (D) of one or more nucleotides. In some embodiments, the one or more ancestry-specific genetic variants, or the one or more individual-specific genetic variants comprise a Copy Number Variant (CNV). In some embodiments, the one or more units of risk comprises an insertion or a deletion of a nucleic acid sequence. In some embodiments, the nucleic acid sequence comprises about two, three, four, five, six, seven, eight, nine, or ten, nucleotides. In some embodiments, the nucleic acid sequence comprises more than three nucleotides. In some embodiments, the nucleic acid sequence comprises an entire gene. In some embodiments, the methods further comprise providing a notification to the individual of the risk that the individual has, or will develop, the specific trait. In some embodiments, the specific trait comprises a nutritional trait, a clinical trait, a subclinical trait, a physical exercise trait, a skin trait, a hair trait, an allergy trait, or a mental trait. In some embodiments, the clinical trait comprises a disease or condition. In some embodiments, the subclinical trait comprises a phenotype of a disease or condition. In some embodiments, the physical exercise trait comprises exercise aversion, aerobic performance, difficulty losing weight, endurance, power, fitness benefits, reduced heart beat response to exercise, lean body mass, muscle soreness, muscle damage risk, muscle repair impairment, stress fracture, overall injury risk, potential for obesity, or resting metabolic rate impairment. In some embodiments, the skin trait comprises collagen breakdown, dryness, antioxidant deficiency, detoxification impairment, skin glycation, pigmented spots, youthfulness, photoaging, dermal sensitivity, or sensitivity to sun. In some embodiments, the nutritional trait comprises vitamin deficiency, mineral deficiency, antioxidant deficiency, fatty acid deficiency, metabolic imbalance, metabolic impairment, metabolic sensitivity, allergy, satiety, or the effectiveness of a healthy diet. In some embodiments, the hair trait comprises hair thickness, hair thinning, hair loss, baldness, oiliness, dryness, dandruff, or hair volume. In some embodiments, the vitamin deficiency comprises a deficiency of a vitamin comprising Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, and Vitamin K. In some embodiments, the mineral deficiency comprises a deficiency of a mineral comprising calcium, iron, magnesium, zinc, or selenium. In some embodiments, the antioxidant deficiency comprises a deficiency of an antioxidant comprising glutathione, or coenzyme Q10 (CoQ10). In some embodiments, the fatty acid deficiency comprises a deficiency in polyunsaturated fatty acids or monounsaturated fatty acids. In some embodiments, the metabolic imbalance comprises glucose imbalance. In some embodiments, the metabolic impairment comprises impaired metabolism of caffeine or drug therapy. In some embodiments, the metabolic sensitivity comprises gluten sensitivity, glycan sensitivity, or lactose sensitivity. In some embodiments, the allergy comprises an allergy to food (food allergy) or environmental factors (environmental allergy). In some embodiments, the methods further comprise administering a treatment to the individual effective to ameliorate or prevent the specific trait in the individual, provided the genetic risk score indicates a high likelihood that the individual has, or will develop, the specific trait. In some embodiments, the treatment comprises a supplement or drug therapy. In some embodiments, the supplement comprises a vitamin, mineral, probiotic, anti-oxidant, anti-inflammatory, or combination thereof. In some embodiments, the genetic risk score is calculated by: a) calculating a raw score comprising a total number of the one or more units of risk for each ancestry-specific genetic variant for each subject of the subject group, thereby generating an ancestry-specific observed range of raw scores; b) calculating a total number of the one or more units of risk for each of the one or more individual-specific genetic variants, thereby generating an individual raw score; and c) comparing the individual raw score with the ancestry-specific observed range to generate the genetic risk score. In some embodiments, the genetic risk score is calculated by: a) determining an odds ratio for each of the ancestry-specific genetic risk variants; and b) if two or more ancestry-specific genetic variants are selected, then multiplying the odds ratio for each of the two or more ancestry-specific genetic variants together. In some embodiments, the genetic risk score is calculated by: a) determining a relative risk for each of the ancestry-specific genetic risk variants; and b) if two or more ancestry-specific genetic variants are selected, then multiplying the relative risks for each of the two or more ancestry-specific genetic variants together. In some embodiments, the predetermined genetic variant is determined by a) providing unphased genotype data from an individual; b) phasing the unphased genotype data to generate individual-specific phased haplotypes based on the ancestry of the individual; c) imputing individual-specific genotypes not present in the phased individual-specific phased haplotypes using phased haplotype data from a reference group that has the same ancestry as the individual; and d) selecting a genetic variant from the imputed individual-specific genotypes that is in linkage disequilibrium (LD) an individual-specific genetic variant associated with a likelihood that the individual has, or will develop, a specific trait.

Disclosed herein, in certain embodiments, are wellness reporting systems comprising: a) a computing device comprising at least one processor, a memory, and a software program including instructions executable by at least one processor to assess a likelihood that an individual has, or will develop, a specific trait, the instructions comprising the steps of: (i) providing the genotype of the individual, the genotype comprising one or more individual-specific genetic variants; (ii) assigning an ancestry to the individual based, at least in part, on the genotype of the individual; (iii) using a trait-associated variants database comprising ancestry-specific genetic variants derived from subjects with the same ancestry as the individual (subject group) to select one or more ancestry-specific genetic variants based, at least in part, on the ancestry of the individual, wherein each of the one or more ancestry-specific genetic variants correspond to: (1) an individual-specific genetic variant of the one or more individual-specific genetic variants, or (2) a predetermined genetic variant in a linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk; and (iv) calculating a genetic risk score for the individual based on the selected one or more ancestry-specific genetic variants, wherein the genetic risk score is indicative of the likelihood that the individual has, or will develop the specific trait; b) a reporting module generate a report comprising the genetic risk score of the individual for the specific trait; and c) an output module configured to display the report to the individual. In some embodiments, the genetic risk score comprises a percentile or z-score. In some embodiments, the LD is defined by (i) D′ value of at least about 0.20, or (ii) an r² value of at least about 0.70. In some embodiments, the LD is defined by a D′ value comprising between about 0.20 and 0.25, 0.25 and 0.30, 0.30 and 0.35, 0.35 and 0.40, 0.40 and 0.45, 0.45 and 0.50, 0.50 and 0.55, 0.55 and 0.60, 0.60 and 0.65, 0.65 and 0.70, 0.70 and 0.75, 0.75 and 0.80, 0.80 and 0.85, 0.85 and 0.90, 0.90 and 0.95, or 0.95 and 1.0. In some embodiments, the LD is defined by a r² value comprising between about 0.70 and 0.75, 0.75 and 0.80, 0.80 and 0.85, 0.85 and 0.90, 0.90 and 0.95, or 0.95 and 1.0. In some embodiments, the LD is defined by a D′ value comprising at least about 0.20, 0.25, 0.30, 0.35, 0.40, 0.45, 0.50, 0.55, 0.60, 0.65, 0.70, 0.75, 0.80, 0.85, 0.85, 0.90, 0.95 and 1.0. In some embodiments, the LD is defined by a r² value comprising at least about 0.70, 0.75, 0.80, 0.85, 0.90, 0.95 and 1.0. In some embodiments, the genotype of the individual is obtained by subjecting, or having subjected, genetic material obtained from the individual to a genotyping assay. In some embodiments, genotype of the individual is obtained by subjecting the genetic material obtained from the individual to a deoxyribonucleic acid (DNA) array, ribonucleic acid (RNA) array, sequencing assay, or a combination thereof. In some embodiments, the sequencing assay comprises next generation sequencing (NGS). In some embodiments, the methods further comprise updating the trait-associated variants database with the assigned ancestry, a specific trait, and the genotype of the individual. In some embodiments, ancestry is assigned to the individual in (b) using a principle component analysis (PCA), or a maximum likelihood estimation (MLE), or a combination thereof. In some embodiments, the one or more ancestry-specific genetic variants, the one or more individual-specific genetic variants, and the genetic variants in LD with the one or more individual-specific genetic variants comprise a Single Nucleotide Variant (SNV). In some embodiments, the one or more units of risk comprises a risk allele. In some embodiments, the one or more ancestry-specific genetic variants, the one or more individual-specific genetic variants, and the genetic variants in LD with the one or more individual-specific genetic variants comprise an indel characterized by an insertion or a deletion of one or more nucleotides. In some embodiments, the one or more units of risk comprises a insertion (I) or a deletion (D) of one or more nucleotides. In some embodiments, the one or more ancestry-specific genetic variants, or the one or more individual-specific genetic variants comprise a Copy Number Variant (CNV). In some embodiments, the one or more units of risk comprises an insertion or a deletion of a nucleic acid sequence. In some embodiments, the nucleic acid sequence comprises about two, three, four, five, six, seven, eight, nine, or ten, nucleotides. In some embodiments, the nucleic acid sequence comprises more than three nucleotides. In some embodiments, the nucleic acid sequence comprises an entire gene. In some embodiments, the methods further comprise providing a notification to the individual of the risk that the individual has, or will develop, the specific trait. In some embodiments, the specific trait comprises a nutritional trait, a clinical trait, a subclinical trait, a physical exercise trait, a skin trait, a hair trait, an allergy trait, or a mental trait. In some embodiments, the clinical trait comprises a disease or condition. In some embodiments, the subclinical trait comprises a phenotype of a disease or condition. In some embodiments, the physical exercise trait comprises exercise aversion, aerobic performance, difficulty losing weight, endurance, power, fitness benefits, reduced heart beat response to exercise, lean body mass, muscle soreness, muscle damage risk, muscle repair impairment, stress fracture, overall injury risk, potential for obesity, or resting metabolic rate impairment. In some embodiments, the skin trait comprises collagen breakdown, dryness, antioxidant deficiency, detoxification impairment, skin glycation, pigmented spots, youthfulness, photoaging, dermal sensitivity, or sensitivity to sun. In some embodiments, the hair trait comprises hair thickness, hair thinning, hair loss, baldness, oiliness, dryness, dandruff, or hair volume. In some embodiments, the nutritional trait comprises vitamin deficiency, mineral deficiency, antioxidant deficiency, fatty acid deficiency, metabolic imbalance, metabolic impairment, metabolic sensitivity, allergy, satiety, or the effectiveness of a healthy diet. In some embodiments, the vitamin deficiency comprises a deficiency of a vitamin comprising Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, and Vitamin K. In some embodiments, the mineral deficiency comprises a deficiency of a mineral comprising calcium, iron, magnesium, zinc, or selenium. In some embodiments, the antioxidant deficiency comprises a deficiency of an antioxidant comprising glutathione, or coenzyme Q10 (CoQ10). In some embodiments, the fatty acid deficiency comprises a deficiency in polyunsaturated fatty acids or monounsaturated fatty acids. In some embodiments, the metabolic imbalance comprises glucose imbalance. In some embodiments, the metabolic impairment comprises impaired metabolism of caffeine or drug therapy. In some embodiments, the metabolic sensitivity comprises gluten sensitivity, glycan sensitivity, or lactose sensitivity. In some embodiments, the allergy comprises an allergy to food (food allergy) or environmental factors (environmental allergy). In some embodiments, the methods further comprise administering a treatment to the individual effective to ameliorate or prevent the specific trait in the individual, provided the genetic risk score indicates a high likelihood that the individual has, or will develop, the specific trait. In some embodiments, the treatment comprises a supplement or drug therapy. In some embodiments, the supplement comprises a vitamin, mineral, probiotic, anti-oxidant, anti-inflammatory, or combination thereof. In some embodiments, the instructions further comprise a survey to the individual comprising one or more questions relating to the specific trait. In some embodiments, the instructions further comprise receiving, from the individual, one or more answers to one or more questions relating to the specific trait in a survey provided to the individual. In some embodiments, the instructions further comprise: (i) providing a survey to the individual comprising one or more questions relating to the specific trait; and (ii) receiving, from the individual, one or more answers to the one or more questions. In some embodiments, the instructions further comprise storing, in a trait-associated variants database, the ancestry-specific genetic variants associated with the specific trait derived from the subject group. In some embodiments, the output module is configured to display the report on a user interface of a personal electronic device. In some embodiments, the system further comprises a personal electronic device with an application configured to communicate with the output module via a computer network to access the report. In some embodiments, the genetic risk score is calculated by: (1) calculating a raw score comprising a total number of the one or more units of risk for each ancestry-specific genetic variant for each subject of the subject group, thereby generating an ancestry-specific observed range of raw scores; (2) calculating a total number of the one or more units of risk for each of the one or more individual-specific genetic variants, thereby generating an individual raw score; and (3) comparing the individual raw score with the ancestry-specific observed range to generate the genetic risk score. In some embodiments, the genetic risk score is calculated by: (1) determining an odds ratio for each of the ancestry-specific genetic risk variants; and (2) if two or more ancestry-specific genetic variants are selected, then multiplying the odds ratio for each of the two or more ancestry-specific genetic variants together. In some embodiments, the system further comprises the steps of determining the predetermined genetic variant by: a) providing unphased genotype data from an individual; b) phasing the unphased genotype data to generate individual-specific phased haplotypes based on the ancestry of the individual; c) imputing individual-specific genotypes not present in the phased individual-specific phased haplotypes using phased haplotype data from a reference group that has the same ancestry as the individual; and d) selecting a genetic variant from the imputed individual-specific genotypes that is in linkage disequilibrium (LD) the individual-specific genetic variant associated with a likelihood that the individual has, or will develop, a specific trait.

Disclosed herein, in certain embodiments, are non-transitory computer readable storage media, comprising computer-executable code configured to cause at least one processor to perform steps of: a) providing the genotype of the individual, the genotype comprising one or more individual-specific genetic variants; b) assigning an ancestry to the individual based, at least in part, on the genotype of the individual; c) using a trait-associated variants database comprising ancestry-specific genetic variants derived from subjects with the same ancestry as the individual (subject group) to select one or more ancestry-specific genetic variants based, at least in part, on the ancestry of the individual, wherein each of the one or more ancestry-specific genetic variants correspond to: (ii) an individual-specific genetic variant of the one or more individual-specific genetic variants, or (ii) a predetermined genetic variant in a linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk; and d) calculating a genetic risk score for the individual based on the selected one or more ancestry-specific genetic variants, wherein the genetic risk score is indicative of the likelihood that the individual has, or will develop the specific trait. In some embodiments, the media further comprises providing a survey to the individual comprising one or more questions relating to the specific trait. In some embodiments, the media further comprises receiving, from the individual, one or more answers to one or more questions relating to the specific trait in a survey provided to the individual. In some embodiments, the media further comprises: a) providing a survey to the individual comprising one or more questions relating to the specific trait; and c) receiving, from the individual, one or more answers to the one or more questions. In some embodiments, the media further comprising storing, in a trait-associated variants database, the ancestry-specific genetic variants associated with the specific trait derived from the subject group. In some embodiments, the genetic risk score comprises a percentile or z-score. In some embodiments, the LD is defined by (i) D′ value of at least about 0.20, or (ii) an r² value of at least about 0.70. In some embodiments, the LD is defined by a D′ value comprising between about 0.20 and 0.25, 0.25 and 0.30, 0.30 and 0.35, 0.35 and 0.40, 0.40 and 0.45, 0.45 and 0.50, 0.50 and 0.55, 0.55 and 0.60, 0.60 and 0.65, 0.65 and 0.70, 0.70 and 0.75, 0.75 and 0.80, 0.80 and 0.85, 0.85 and 0.90, 0.90 and 0.95, or 0.95 and 1.0. In some embodiments, the LD is defined by a r² value comprising between about 0.70 and 0.75, 0.75 and 0.80, 0.80 and 0.85, 0.85 and 0.90, 0.90 and 0.95, or 0.95 and 1.0. In some embodiments, the LD is defined by a D′ value comprising at least about 0.20, 0.25, 0.30, 0.35, 0.40, 0.45, 0.50, 0.55, 0.60, 0.65, 0.70, 0.75, 0.80, 0.85, 0.85, 0.90, 0.95 and 1.0. In some embodiments, the LD is defined by a r² value comprising at least about 0.70, 0.75, 0.80, 0.85, 0.90, 0.95 and 1.0. In some embodiments, the genotype of the individual is obtained by subjecting, or having subjected, genetic material obtained from the individual to a genotyping assay. In some embodiments, genotype of the individual is obtained by subjecting the genetic material obtained from the individual to a deoxyribonucleic acid (DNA) array, ribonucleic acid (RNA) array, sequencing assay, or a combination thereof. In some embodiments, the sequencing assay comprises next generation sequencing (NGS). In some embodiments, the methods further comprise updating the trait-associated variants database with the assigned ancestry, a specific trait, and the genotype of the individual. In some embodiments, ancestry is assigned to the individual in (b) using a principle component analysis (PCA), or a maximum likelihood estimation (MLE), or a combination thereof. In some embodiments, the one or more ancestry-specific genetic variants, the one or more individual-specific genetic variants, and the genetic variants in LD with the one or more individual-specific genetic variants comprise a Single Nucleotide Variant (SNV). In some embodiments, the one or more units of risk comprises a risk allele. In some embodiments, the one or more ancestry-specific genetic variants, the one or more individual-specific genetic variants, and the genetic variants in LD with the one or more individual-specific genetic variants comprise an indel characterized by an insertion or a deletion of one or more nucleotides. In some embodiments, the one or more units of risk comprises a insertion (I) or a deletion (D) of one or more nucleotides. In some embodiments, the one or more ancestry-specific genetic variants, or the one or more individual-specific genetic variants comprise a Copy Number Variant (CNV). In some embodiments, the one or more units of risk comprises an insertion or a deletion of a nucleic acid sequence. In some embodiments, the nucleic acid sequence comprises about two, three, four, five, six, seven, eight, nine, or ten, nucleotides. In some embodiments, the nucleic acid sequence comprises more than three nucleotides. In some embodiments, the nucleic acid sequence comprises an entire gene. In some embodiments, the methods further comprise providing a notification to the individual of the risk that the individual has, or will develop, the specific trait. In some embodiments, the specific trait comprises a nutritional trait, a clinical trait, a subclinical trait, a physical exercise trait, a skin trait, a hair trait, an allergy trait, or a mental trait. In some embodiments, the clinical trait comprises a disease or condition. In some embodiments, the subclinical trait comprises a phenotype of a disease or condition. In some embodiments, the physical exercise trait comprises exercise aversion, aerobic performance, difficulty losing weight, endurance, power, fitness benefits, reduced heart beat response to exercise, lean body mass, muscle soreness, muscle damage risk, muscle repair impairment, stress fracture, overall injury risk, potential for obesity, or resting metabolic rate impairment. In some embodiments, the skin trait comprises collagen breakdown, dryness, antioxidant deficiency, detoxification impairment, skin glycation, pigmented spots, youthfulness, photoaging, dermal sensitivity, or sensitivity to sun. In some embodiments, the hair trait comprises hair thickness, hair thinning, hair loss, baldness, oiliness, dryness, dandruff, or hair volume. In some embodiments, the nutritional trait comprises vitamin deficiency, mineral deficiency, antioxidant deficiency, fatty acid deficiency, metabolic imbalance, metabolic impairment, metabolic sensitivity, allergy, satiety, or the effectiveness of a healthy diet. In some embodiments, the vitamin deficiency comprises a deficiency of a vitamin comprising Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, and Vitamin K. In some embodiments, the mineral deficiency comprises a deficiency of a mineral comprising calcium, iron, magnesium, zinc, or selenium. In some embodiments, the antioxidant deficiency comprises a deficiency of an antioxidant comprising glutathione, or coenzyme Q10 (CoQ10). In some embodiments, the fatty acid deficiency comprises a deficiency in polyunsaturated fatty acids or monounsaturated fatty acids. In some embodiments, the metabolic imbalance comprises glucose imbalance. In some embodiments, the metabolic impairment comprises impaired metabolism of caffeine or drug therapy. In some embodiments, the metabolic sensitivity comprises gluten sensitivity, glycan sensitivity, or lactose sensitivity. In some embodiments, the allergy comprises an allergy to food (food allergy) or environmental factors (environmental allergy). In some embodiments, the methods further comprise administering a treatment to the individual effective to ameliorate or prevent the specific trait in the individual, provided the genetic risk score indicates a high likelihood that the individual has, or will develop, the specific trait. In some embodiments, the treatment comprises a supplement or drug therapy. In some embodiments, the supplement comprises a vitamin, mineral, probiotic, anti-oxidant, anti-inflammatory, or combination thereof. In some embodiments, the genetic risk score is calculated by: (1) calculating a raw score comprising a total number of the one or more units of risk for each ancestry-specific genetic variant for each subject of the subject group, thereby generating an ancestry-specific observed range of raw scores; (2) calculating a total number of the one or more units of risk for each of the one or more individual-specific genetic variants, thereby generating an individual raw score; and (3) comparing the individual raw score with the ancestry-specific observed range to generate the genetic risk score. In some embodiments, the genetic risk score is calculated by: (1) determining an odds ratio for each of the ancestry-specific genetic risk variants; and (2) if two or more ancestry-specific genetic variants are selected, then multiplying the odds ratio for each of the two or more ancestry-specific genetic variants together. In some embodiments, the wherein the computer-executable code is further configured to cause at least one processor to perform step of determining the predetermined genetic variant by: a) providing unphased genotype data from an individual; b) phasing the unphased genotype data to generate individual-specific phased haplotypes based on the ancestry of the individual; c) imputing individual-specific genotypes not present in the phased individual-specific phased haplotypes using phased haplotype data from a reference group that has the same ancestry as the individual; and d) selecting a genetic variant from the imputed individual-specific genotypes that is in linkage disequilibrium (LD) the individual-specific genetic variant associated with a likelihood that the individual has, or will develop, a specific trait.

### BRIEF DESCRIPTION OF THE DRAWINGS

FIG. 1 is a block diagram illustrating an exemplary system for determining the ancestry-specific genetic risk score for an individual.

FIG. 2 is a flowchart illustrating an exemplary process for determining a genetic risk score for an individual.

FIG. 3 is a flow chart illustrating an exemplary process for determining the ancestry-specific genetic risk score for an individual using one or more reference genetic variants.

FIG. 4 is a flow chart illustrating an exemplary process for determining the ancestry-specific genetic risk score for an individual using one or more ancestry-specific genetic variants from the trait-associated database.

FIG. 5 is a flow chart illustrating an exemplary process for determining the ancestry-specific genetic risk score for an individual using one or more ancestry-specific genetic variants from the trait-associated database.

FIG. 6A-FIG. 6F exemplifies a report according to the present embodiments, in which the GRS for multiple specific phenotypic traits is displayed to the subject. FIG. 6A exemplifies a report with a summary of fitness phenotypic traits. FIG. 6B exemplifies behavior recommendations related to the fitness phenotype trait: potential for obesity. FIG. 6C exemplifies a report with a summary of skin phenotypic traits. FIG. 6D exemplifies behavior recommendations related to the skin phenotype trait: antioxidant deficiency. FIG. 6E exemplifies a report with a summary of nutrition phenotypic traits. FIG. 6F exemplifies behavior recommendations related to the nutrition phenotype trait: impaired satiety.

FIG. 7A-FIG. 7D exemplifies a nutrient report focusing on nutrition and health according to the present embodiments, in which the GRS for multiple specific phenotypic traits is displayed to the subject. FIG. 7A exemplifies a report with a summary of food sensitivities. FIG. 7B exemplifies a report with a summary of mineral and nutrient deficiencies. FIG. 7C exemplifies a report with a summary of diet management phenotypes. FIG. 7D exemplifies a report with a summary of vitamin deficiencies.

### DETAILED DESCRIPTION OF THE INVENTION

It is believed that differences in haplotype heterogeneity, as well as recombination rates, contribute significantly to the variance found in linkage disequilibrium (LD) between different ancestral populations. Current genetic risk prediction methods fail to account for the ancestry of the subject group when selecting a proxy genetic variant, which results in selection of a poor indicator of risk in given population. The methods, media, and systems disclosed herein, provide a solution to this problem, by selecting a proxy genetic variant based on LD within the particular ancestral population of which the individual belongs. Further, the methods, media, and systems disclosed herein utilize a software program configured to use predetermined LD patterns, which may be leveraged when calculating a genetic risk score (GRS) for which an individual-specific genetic variant was previously undisclosed. Thus, the present solution, disclosed herein, increases the accuracy and efficiency of a genetic risk prediction, as compared to existing methods.

Current risk prediction methods do not utilize ancestry-specific LD information. However, whether a genetic variant is in LD with another genetic variant is heavily influenced by what ancestral population is studied. In a non-limiting example, two genetic variants that are in LD in a predominantly Caucasian population may not necessarily be in LD in, for example, a Chinese population. The inverse may also be true. Taking into account ancestry-specific LD patterns when calculating a GRS for an individual is advantageous over the state of the art for many reasons including, but not limited to, (i) avoidance of errors (e.g., the two genetic variants are not in LD within that population at all), and (ii) avoidance of counting of a genetic variant more than once. Taking into account ancestry-specific LD patterns yields more accurate GRS predictions by ensuring genetic risk variants in LD are identified, and preventing inflation of a GRS caused by counting a single genetic variant more than once.

Disclosed herein in some embodiments are genetic risk prediction methods, media, and systems for calculating a genetic risk score (GRS) representing a likelihood that an individual will develop a specific phenotype trait, based on the ancestry of the individual. In some embodiments, the GRS is calculated based on a number and type of genetic variants making up the genotype of the individual detected in a sample obtained from the individual, as compared to a subject population of the same ancestry as the individual. In some embodiments, ancestry of the individual is determined by analysis of the genotype of the individual. Also disclosed herein, are methods, media, and systems for recommending a behavioral modification related to the specific phenotypic trait to the individual, based on the calculated GRS for that trait.

### Genotypes and Genetic Variants

Genome-wide association studies (GWAS) consider hundreds of thousands of genetic variants, including single nucleotide variants, (SNVs), insertions/deletions (indels), and copy-number variants (CNVs) to identify associations between genetic variants within a population and complex clinical conditions and phenotypic traits. Detecting genetic variants associated with specific phenotypic traits in a sample obtained from an individual is considered indicative that the individual has, or will develop, the specific phenotypic trait. In some embodiments, the individual obtains his or her own sample, and provides the sample to a laboratory for processing and analysis. In some embodiments, genetic material is extracted from the sample obtained from the subject. In some embodiments, genetic variants are detected in the genetic material from the sample obtained from an individual using a genotyping assay (e.g., genotyping array, quantitative polymerase chain reaction (qPCR), and/or fluorogenic qPCR). In some embodiments, the genetic information is analyzed to determine the ancestry of the individual.

A genetic variant (e.g., SNV, SNP, indel, CNV) may fall within coding regions of a gene, a non-coding region of a gene, or in an intergenic region between genes. A genetic variant within a coding region of a gene may, or may not, result in a different protein isoform produced due to redundancy in the genetic code. A genetic variant within a non-coding region or intergenic region of a gene may influence the expression and/or activity of the gene, or gene expression products expressed from the gene.

Disclosed herein in some embodiments are methods and systems for determining the genotype of an individual. In some embodiments, the individual is suffering from a disease or condition, or symptoms related to the disease or condition. In some embodiments, the disease or condition comprises a deficiency disease, a hereditary disease, or psychological disease. In some embodiments the disease or condition comprises an immunological disease and/or a metabolic disease. In some embodiments, the immunological disease comprises an autoimmune disease or disorder. Non-limiting examples of an autoimmune disease or disorder include Grave's disease, Hashimoto's thyroiditis, systemic lupus erythematosus (lupus), multiple sclerosis, rheumatoid arthritis, inflammatory bowel disease, Crohn's disease, ulcerative colitis, and cancer. Non-limiting examples of metabolic diseases or conditions include Type 1 diabetes, Type 2, diabetes, diseases affecting absorption of macronutrients (e.g., amino acids, carbohydrates, or lipids), diseases affecting absorption of micronutrients (e.g., vitamins or minerals), diseases affecting mitochondrial function, diseases affecting liver function (e.g., nonalcoholic fatty liver diseases), and diseases affecting kidney function.

Disclosed herein in some embodiments are methods and systems for calculating a genetic risk score (GRS) representing a likelihood that an individual has, or will develop, a specific phenotypic trait, using the genotype and/or genetic variants disclosed herein. In some embodiments, a single genetic variant is used. In some embodiments, two genetic variants are used. In some embodiments, three genetic variants are used. In some embodiments, four genetic variants are used. In some embodiments, five genetic variants are used. In some embodiments, six genetic variants are used. In some embodiments, seven genetic variants are used. In some embodiments, eight genetic variants are used. In some embodiments, nine genetic variants are used. In some embodiments, ten genetic variants are used. In some embodiments, at least about two genetic variants are used. In some embodiments, at least about three genetic variants are used. In some embodiments, at least about four genetic variants are used. In some embodiments, at least about five genetic variants are used. In some embodiments, at least about six genetic variants are used. In some embodiments, at least about seven genetic variants are used. In some embodiments, at least about eight genetic variants are used. In some embodiments, at least about nine genetic variants are used. In some embodiments, at least about ten genetic variants are used. In some embodiments, two genetic variants are used.

Disclosed herein, in some embodiments, are genotypes comprising one or more genetic variants (e.g., indel, SNV, SNP) provided in one or more of SEQ ID NOS: 1-218 that are used in the methods, systems and kits described herein. In some embodiments, the genotypes described herein comprise a single genetic variant. In some embodiments, the genotypes comprise two genetic variants. In some embodiments, the genotypes comprise three genetic variants. In some embodiments, the genotypes comprise four genetic variants. In some embodiments, the genotypes comprise five genetic variants. In some embodiments, the genotypes comprise six genetic variants. In some embodiments, the genotypes comprise seven genetic variants. In some embodiments, the genotypes comprise eight genetic variants. In some embodiments, the genotypes comprise nine genetic variants. In some embodiments, the genotypes comprise ten genetic variants. In some embodiments, the genotypes comprise more than ten genetic variants.

In some embodiments, the genotypes comprise at least about two genetic variants. In some embodiments, the genotypes comprise at least about three genetic variants. In some embodiments, the genotypes comprise at least about four genetic variants. In some embodiments, the genotypes comprise at least about five genetic variants. In some embodiments, the genotypes comprise at least about six genetic variants. In some embodiments, the genotypes comprise at least about seven genetic variants. In some embodiments, the genotypes comprise at least about eight genetic variants. In some embodiments, the genotypes comprise at least about nine genetic variants. In some embodiments, the genotypes comprise at least about ten genetic variants.

In some embodiments, at least one genetic variant listed in any one of Table 1-Table 44 is used. In some embodiments, the genetic variants are used using the methods of detection disclosed herein. In some embodiments, the methods and systems described herein use (e.g., detect, analyze) one or more genetic variants provided in Table 1. In some embodiments, the methods and systems described herein use one or more genetic variants provided in Table 2. In some embodiments, the methods and systems described herein use one or more genetic variants provided in Table 3. In some embodiments, the methods and systems described herein use one or more genetic variants provided in Table 4. In some embodiments, the methods and systems described herein use one or more genetic variants provided in Table 5. In some embodiments, the methods and systems described herein use one or more genetic variants provided in Table 6. In some embodiments, the methods and systems described herein use one or more genetic variants provided in Table 7. In some embodiments, the methods and systems described herein use one or more genetic variants provided in Table 8. In some embodiments, the methods and systems described herein use one or more genetic variants provided in Table 9. In some embodiments, the methods and systems described herein use one or more genetic variants provided in Table 10. In some embodiments, the methods and systems described herein use one or more genetic variants provided in Table 11. In some embodiments, the methods and systems described herein use one or more genetic variants provided in Table 12. In some embodiments, the methods and systems described herein use one or more genetic variants provided in Table 13. In some embodiments, the methods and systems described herein use one or more genetic variants provided in Table 14. In some embodiments, the methods and systems described herein use one or more genetic variants provided in Table 15. In some embodiments, the methods and systems described herein use one or more genetic variants provided in Table 16. In some embodiments, the methods and systems described herein use one or more genetic variants provided in Table 17. In some embodiments, the methods and systems described herein use one or more genetic variants provided in Table 18. In some embodiments, the methods and systems described herein use one or more genetic variants provided in Table 19. In some embodiments, the methods and systems described herein use one or more genetic variants provided in Table 20. In some embodiments, the methods and systems described herein use one or more genetic variants provided in Table 21. In some embodiments, the methods and systems described herein use one or more genetic variants provided in Table 22. In some embodiments, the methods and systems described herein use one or more genetic variants provided in Table 23. In some embodiments, the methods and systems described herein use one or more genetic variants provided in Table 24. In some embodiments, the methods and systems described herein use one or more genetic variants provided in Table 25. In some embodiments, the methods and systems described herein use one or more genetic variants provided in Table 26. In some embodiments, the methods and systems described herein use one or more genetic variants provided in Table 27. In some embodiments, the methods and systems described herein use one or more genetic variants provided in Table 28. In some embodiments, the methods and systems described herein use one or more genetic variants provided in Table 29. In some embodiments, the methods and systems described herein use one or more genetic variants provided in Table 30. In some embodiments, the methods and systems described herein use one or more genetic variants provided in Table 31. In some embodiments, the methods and systems described herein use one or more genetic variants provided in Table 32. In some embodiments, the methods and systems described herein use one or more genetic variants provided in Table 33. In some embodiments, the methods and systems described herein use one or more genetic variants provided in Table 34. In some embodiments, the methods and systems described herein use one or more genetic variants provided in Table 35. In some embodiments, the methods and systems described herein use one or more genetic variants provided in Table 36. In some embodiments, the methods and systems described herein use one or more genetic variants provided in Table 37. In some embodiments, the methods and systems described herein use one or more genetic variants provided in Table 38. In some embodiments, the methods and systems described herein use one or more genetic variants provided in Table 39. In some embodiments, the methods and systems described herein use one or more genetic variants provided in Table 40. In some embodiments, the methods and systems described herein use one or more genetic variants provided in Table 41. In some embodiments, the methods and systems described herein use one or more genetic variants provided in Table 42. In some embodiments, the methods and systems described herein use one or more genetic variants provided in Table 43. In some embodiments, the methods and systems described herein use one or more genetic variants provided in Table 44.

In some embodiments, the methods, systems, and kits utilize the major or the minor allele in the one or more genetic variants. In some embodiments, the minor allele is used. In some embodiments, the major allele is used. In some embodiments, the methods, systems, and kits utilize the nucleotide represented by a non-nucleic acid letter or code. In some cases, the non-nucleic acid letter or code is an International Union of Pure and Applied Chemistry (IUPAC) nucleotide code. provided in any one or SEQ ID NOS: 1-218. A genetic variant provided in any one of Table 1-Table 44 has a corresponding SEQ ID NO that provides a nucleic acid sequence comprising the nucleotide or polynucleotide sequence that is associated with a risk of having or developing the specific phenotypic trait.

Methods and systems disclosed herein are generally suitable for analyzing a sample obtained from an individual. Similarly, methods disclosed herein comprises processing and/or analysis of the sample. In some instances, the sample is obtained directly, or indirectly, from the individual. In some instances, the sample is obtained by a fluid draw, swab or fluid collection. In some instances, the sample comprises whole blood, peripheral blood, plasma, serum, saliva, cheek swab, urine, or other bodily fluid or tissue.

In some embodiments, the genotype of the individual is determined by subjecting a sample obtained from the individual to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™ or SYBR green), which involves a nucleic acid amplification reaction with a specific primer pair, and hybridization of the amplified nucleic acid probes comprising a detectable moiety or molecule that is specific to a target nucleic acid sequence. An additional exemplary nucleic acid-based detection assay comprises the use of nucleic acid probes conjugated or otherwise immobilized on a bead, multi-well plate, array, or other substrate, wherein the nucleic acid probes are configured to hybridize with a target nucleic acid sequence. In some instances, the nucleic acid probe is specific to a genetic variant (e.g., SNP, SNV, CNV, or indel) is used. In some instances, the nucleic acid probe specific to a SNP or SNV comprises a nucleic acid probe sequence sufficiently complementary to a risk or protective allele of interest, such that hybridization is specific to the risk or protective allele. In some instances, the nucleic acid probe specific to an indel comprises a nucleic acid probe sequence sufficiently complementary to an insertion of a nucleobase within a polynucleotide sequence flanking the insertion, such that hybridization is specific to the indel. In some instances, the nucleic acid probe specific to an indel comprises a probe sequence sufficiently complementary to a polynucleotide sequence flanking a deletion of a nucleobase within the polynucleotide sequence, such that hybridization is specific to the indel. In some instances, a plurality of nucleic acid probes are required to detect a CNV, specific to various regions within a polynucleotide sequence comprising the CNV. In a non-limiting example, a plurality of nucleic acid probes specific to a single exon CNV within a gene may comprise a high-density of between 2 and 3, 3 and 4, 4 and 5, 5 and 6, and 6 and 7 nucleic acid probes, each nucleic acid probe sufficiently complementary to exonic regions of the gene may be used. In another non-limiting example, long CNVs may be detected utilizing a plurality of nucleic acid probes dispersed throughout the genome of the individual.

Exemplary nucleic acid probes useful for detecting the genotypes described here are risk-allele-specific and comprise an oligonucleotide sequence provided in any one of SEQ ID NOS: 1-218. In some cases, the nucleic acid probe is at least 10 but not more than 50 contiguous nucleotides in length. In some cases, the nucleic acid probe is between about 15 and about 55 nucleotides long. In some cases, the nucleic acid probe is between about 10 and about 100 nucleotides long. In some cases, the nucleic acid probe is between about 10 and about 90 nucleotides long. In some cases, the nucleic acid probe is between about 10 and about 80 nucleotides long. In some cases, the nucleic acid probe is between about 10 and about 70 nucleotides long. In some cases, the nucleic acid probe is between about 10 and about 60 nucleotides long. In some cases, the nucleic acid probe is between about 10 and about 50 nucleotides long. In some cases, the nucleic acid probe is between about 10 and about 40 nucleotides long. In some cases, the nucleic acid probe is between about 10 and about 30 nucleotides long. In some cases, the nucleic acid probe is between about 20 and about 60 nucleotides long. In some cases, the nucleic acid probe is between about 25 and about 65 nucleotides long. In some cases, the nucleic acid probe is between about 30 and about 70 nucleotides long. In some cases, the nucleic acid probe is between about 35 and about 75 nucleotides long. In some cases, the nucleic acid probe is between about 40 and about 70 nucleotides long.

In some embodiments, the methods of detecting a genotype of an individual comprise subjecting a sample obtained from the individual to a nucleic acid amplification assay. In some instances, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising the risk variant (e.g., SNP, SNV, CNV, or indel). In some instances, the amplification assays requires primers. The known nucleic acid sequence for the genes, or genetic variants, within the genotype is sufficient to enable one of skill in the art to select primers to amplify any portion of the gene or genetic variants. A DNA sample suitable as a primer may be obtained, e.g., by PCR amplification of genomic DNA, fragments of genomic DNA, fragments of genomic DNA ligated to adaptor sequences or cloned sequences. Any suitable computer program can be used to design of primers with the desired specificity and optimal amplification properties, such as Oligo version 7.0 (National Biosciences). Exemplary primers useful for amplifying the genotypes described herein are least 10 and not more than 30 nucleotides in length and comprise a nucleic acid sequence that flanks the indel, SNV, SNP, or CNV of interest provided in one or more of SEQ ID NOS: 1-218.

In some embodiments, detecting the presence or absence of a genotype comprises sequencing genetic material from a sample obtained from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modem sequencing technologies such as Illumina sequencing (e.g., Solexa), Roche 454 sequencing, Ion Torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In some instances, the nucleic acid sequence of the genotype comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Determining a Likelihood that an Individual has, or Will Develop a Specific Phenotypic Trait

Aspects disclosed herein provide methods, media, and systems of calculating a genetic risk score (GRS) representing the likelihood that an individual will develop a specific phenotypic trait. In some embodiments, the specific phenotypic trait comprises a phenotypic trait discussed herein, including, but not limited to a clinical trait, a subclinical trait, a physical exercise trait, or a mental trait.

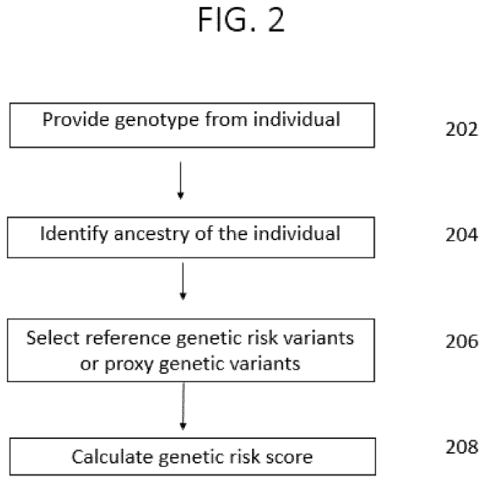

FIG. 2 describes an exemplary workflow to determine a likelihood that an individual has, or will develop, a specific trait by calculating a genetic risk score (GRS). The genotype of the individual is provided 202; the genotype comprising one or more individual-specific genetic variants. Next, the ancestry of the individual is assigned 204 based, at least in part, on the genotype of the individual. Next, one or more reference genetic variants based is selected 206, wherein each of the one or more reference genetic variants correspond to an individual-specific genetic variant of the one or more individual-specific genetic variants or a predetermined genetic variant in a linkage disequilibrium (LD) with an individual-specific genetic variant of the one or more individual-specific genetic variants in a subject population. Next, calculating a genetic risk score for the individual 208 based on the selected one or more reference genetic variants within a subject population, wherein the genetic risk score is indicative of the likelihood that the individual has, or will develop the specific trait. In some instances, the GRS is calculated using any one of the methods disclosed herein.

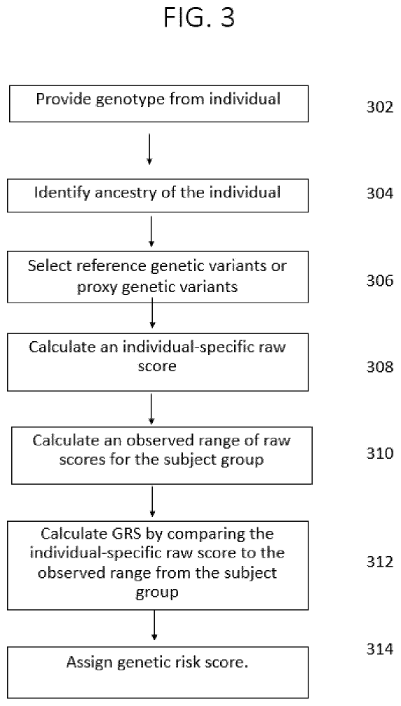

FIG. 3 describes an exemplary workflow to determine a likelihood that an individual has, or will develop, a specific trait based by calculating a GRS as compared to a subject population that is not ancestry specific. The genotype of the individual is provided 302; the genotype comprising one or more individual-specific genetic variants. Next, the ancestry of the individual is assigned based, at least in part, on the genotype of the individual 304. Next, one or more reference genetic variants based is selected 306, wherein each of the one or more reference genetic variants corresponds to an individual-specific genetic variant of the one or more individual-specific genetic variants or a predetermined genetic variant in a linkage disequilibrium (LD) with an individual-specific genetic variant of the one or more individual-specific genetic variants in a subject population. Next an individual-specific raw score is calculated 308. Numerical values are assigned to units of risk within the individual-specific genetic variants, and all numerical values for each individual-specific genetic variant are added together to generate a individual-specific raw score. The same calculations are performed to generate a raw score for each individual within the subject group, thereby generating an observed range of raw scores (observed range) 310. Next, the individual-specific raw score is compared to the observed range to calculate a percentage of risk relative to the subject population 312. Next, a genetic risk score (GRS) is assigned to the individual 314. In some instances, the GRS is in the form as a percentile. In some instances, the percentile is in the form of a z-score.

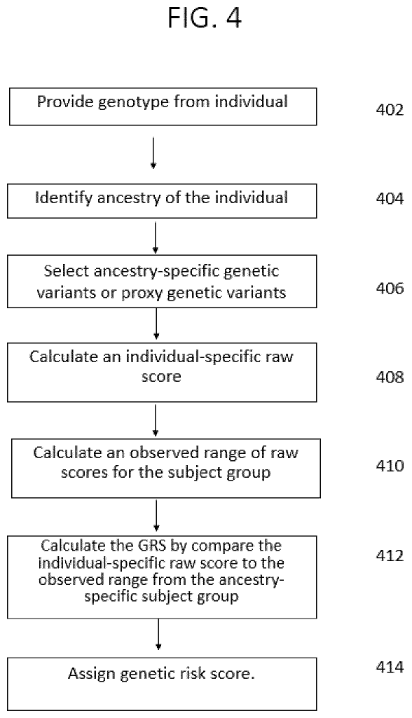

FIG. 4 describes an exemplary workflow to determine a likelihood that an individual has, or will develop, a specific trait based on the ancestry of the individual. The genotype of the individual is provided 402; the genotype comprising one or more individual-specific genetic variants. Next, the ancestry to the individual is assigned 404 based, at least in part, on the genotype of the individual. Next, ancestry-specific genetic variants derived from subjects with the same ancestry as the individual (ancestry-specific subject group) are selected from a trait-associated variants database 406, selected based, at least in part, on the ancestry of the individual, wherein each of the one or more ancestry-specific genetic variants correspond to: (i) an individual-specific genetic variant of the one or more individual-specific genetic variants, or (ii) a predetermined genetic variant in a linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk. Next an individual-specific raw score is calculated 408. Numerical values are assigned to units of risk within the individual-specific genetic variants, and all numerical values for each individual-specific genetic variant are added together to generate an individual-specific raw score. The same calculations are performed to generate a raw score for each individual within the ancestry-specific subject group, thereby generating an observed range of raw scores (observed range) 410. Next, the individual-specific raw score is compared to the ancestry-specific observed range to calculate a percentage of risk relative to the ancestry-specific subject population 412. Next, a genetic risk score (GRS) is assigned to the individual 414. In some instances, the GRS is in the form as a percentile. In some instances, the percentile is in the form of a z-score.

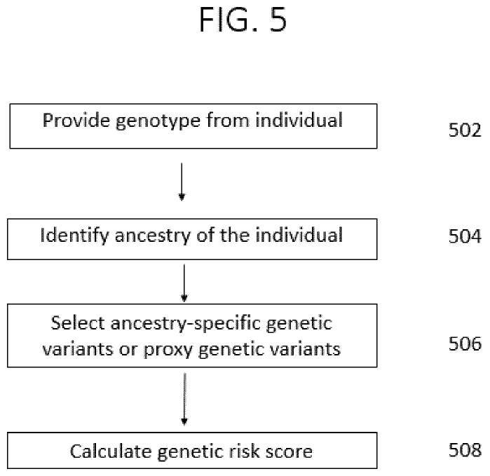

FIG. 5 describes an exemplary workflow to determine a likelihood that an individual has, or will develop, a specific trait based on the ancestry of the individual. The genotype of the individual is provided 502; the genotype comprising one or more individual-specific genetic variants. Next, the ancestry to the individual is assigned 504 based, at least in part, on the genotype of the individual. Next, ancestry-specific genetic variants derived from subjects with the same ancestry as the individual (ancestry-specific subject group) are selected from a trait-associated variants database 506, selected based, at least in part, on the ancestry of the individual, wherein each of the one or more ancestry-specific genetic variants correspond to: (i) an individual-specific genetic variant of the one or more individual-specific genetic variants, or (ii) a predetermined genetic variant in a linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk. Next, a genetic risk score (GRS) for the individual is calculated based on the selected one or more ancestry-specific genetic variants 508, wherein the genetic risk score is indicative of the likelihood that the individual has, or will develop the specific trait. In some instances, the GRS is calculating using any one of the methods disclosed herein.

### Assigning Ancestry of the Individual

In some instances, ancestry is assigned to the individual by analyzing the genotype of the individual. In some instances, the genotype of the individual is analyzed using a method comprising: maximum likelihood or principal component analysis (PCA). In some instances, a computer program comprising SNPRelate, ADMIXTURE, PLINK, or STRUCTURE is used. For example, after PCA has been performed by SNPRelate, the first two principal components (PC1 and PC2) from populations of known ancestry are each combined into a single data point or centroid. An individual ancestry is classified by its proximity to the nearest centroid of known ancestry. This method relies upon the nearest centroid classification model

### Trait-Associated Database

In some embodiments, a trait-associated database is used. In some instances, the trait-associated database comprises a genotype, a phenotype, and/or an ancestry data of the subject group. In some instances, the subject group is derived from a published genome wide association study (GWAS). In some instances, the published GWAS is recorded in a peer-reviewed journal. In some instances, the trait-associated database enables selection of genetic variants present in a subject group of the same ancestry as the individual. In some instances, the trait-associated database is updated with the genotype, phenotype, and/or ancestry data from the individual. Many databases are suitable for storage and retrieval of genotypic, phenotypic data, and ancestry data. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. In some embodiments, a database is connected to a distributed ledger. In some embodiments, the distributed ledger comprises a blockchain. A database may be based on one or more local computer storage devices.

### Selecting One or More Reference Genetic Variants or Ancestry-Specific Genetic Variants

In some embodiments, reference genetic variants or ancestry-specific genetic variants are used to calculate a GRS for an individual. In some instances, the one or more genetic variants comprise reference genetic variants from a subject group of any ancestry. In some embodiments, the subject group comprises individuals of one or more ancestries comprising Japanese, German, Irish, African, South African, English, Mexican, Italian, Polish, French, Native American, Scottish, Dutch, Norwegian, Scotch-Irish, Swedish, Puerto Rican, Russian, Hispanic, French Canadian, Filipino, South Korean, North Korean, Indonesian, Chinese, Taiwanese, Malaysian, Afro-Caribbean, Caucasian, American Indian/Alaskan Native (includes people of Central and South American origin with tribal affiliation), Pacific Islander (includes Hawaii, Guam, Samoa, etc.), South Asian (includes people from Afghanistan, India, Pakistan, Bangladesh, Sri Lanka and Nepal), Japanese, Thai, Indigenous Australian (Aboriginal, Torres Strait Islander). In some instances, the one or more reference genetic variant comprises an ancestry-specific genetic variant derived from a subject group comprising individuals of the same ancestry as the individual (ancestry-specific genetic variants).

In some instances, the reference genetic variants are selected, at least in part, because they are derived from a subject group of the same ancestry as the individual (ancestry-specific genetic variants). In some instances, the ancestry of the individual is determined by analyzing the genotype of the individual using the methods disclosed herein. In some instances, the ancestry-specific genetic variants are selected from the trait-associated variants database disclosed herein.

In some instances, the ancestry-specific genetic variants correspond to the individual-specific genetic variant within the genotype of the individual. In some instances, a corresponding individual-specific genetic variant is unknown, in which case another genetic variant is selected to serve as a proxy for the unknown individual-specific genetic variant.

### Selecting a Proxy Genetic Variant

In some embodiments, proxy genetic variants are used to calculate a GRS when an individual-specific genetic variant is unknown. In some instances, a predetermined genetic variant is selected to serve as the proxy is provided. Disclosed herein, in some embodiments, are methods of predetermining a proxy genetic variant corresponding to an unknown individual-specific genetic variant, the method comprising: (i) providing unphased genotype data from an individual; (ii) phasing the unphased genotype data to generate individual-specific phased haplotypes based on the ancestry of the individual; (iii) imputing individual-specific genotypes not present in the phased individual-specific phased haplotypes using phased haplotype data from a reference group that has the same ancestry as the individual; and (iv) selecting a genetic variant from the imputed individual-specific genotypes that is in linkage disequilibrium (LD) an individual-specific genetic variant associated with a likelihood that the individual has, or will develop, a specific trait.

In some instances, methods comprise selecting an indel (insertion/deletion) as a proxy for an unknown individual-specific indel. In some instances, methods comprises selecting a copy-number variant (CNV) as a proxy for an unknown individual-specific CNV.

“Linkage disequilibrium,” or “LD,” as used herein refers to the non-random association of units of risk with genetic risk variants in a given population. LD may be defined by a D′ value corresponding to the difference between an observed and expected unit of risk frequencies in the population (D=Pab−PaPb), which is scaled by the theoretical maximum value of D. LD may be defined by an r² value corresponding to the difference between an observed and expected unit of risk frequencies in the population (D=Pab−PaPb), which is scaled by the individual frequencies of the different loci. In some embodiments, D′ comprises at least 0.20. In some embodiments, r² comprises at least 0.70. In some embodiments, LD is defined by a D′ value comprising at least about 0.20, 0.25, 0.30, 0.35, 0.40, 0.45, 0.50, 0.55, 0.60, 0.65, 0.70, 0.75, 0.80, 0.85, 0.90, 0.95 or 1. In some embodiments, LD is defined by an r² value comprising at least about 0.70, 0.75, 0.75, 0.80, 0.85, 0.90, 0.95, or 1.0. LD differs amongst subject populations belonging to different ancestries. In a non-limiting example, a SNV in LD with a proxy SNV in a subject population of Chinese individuals may not necessarily be in LD within a subject population of Caucasian individuals. Thus, predetermination of a proxy genetic variant based on ancestry-specific phased haplotype data provides increases accuracy of genetic risk predictions based, at least in part, on the proxy.

### Calculating a Genetic Risk Score

In some embodiments, methods of calculating a genetic risk score (GRS) for the individual based on the ancestry of the individual are provided. The genetic variants disclosed herein comprise SNVs, indels, and/or CNVs. Each genetic variant comprises units of risk used to calculate a GRS. In some instances, a unit of risk within an SNV comprises the risk allele. In some instances, a unit of risk within an indel comprises the insertion or deletion. In some instances, a unit of risk within a CNV comprises an increase or a decrease in a number of copies of a gene or segment of a gene as compared to a wild-type copy number. A person of skill in the art would understand that many methods of calculating a GRS may be used to calculate the GRS of the individual according to the present methods and systems.

Disclosed herein, in some embodiments, are methods of calculating a GRS of an individual. In some instances, the units of risk within an SNV (e.g., risk allele), an Indel (e.g., insertion or deletion), and/or CNV (e.g., copy number) may be assigned an arbitrary numerical value. In a non-limiting example of calculating a GRS involving SNVs, a homozygous genotype for a risk allele within a SNV (RR) is assigned a numerical value 2; a heterozygous genotype for a risk allele within a SN (R) is assigned a numerical value 1; a genotype that is nonrisk (N) is assigned a numerical value 0. Next, each numerical value for all individual SNVs corresponding to an ancestry-specific SNV, are added together, a divided by a total number of genetic variants used in the model, to generate a raw score for the individual (individual raw score). The same calculations are performed for each individual belonging to the subject group, thereby generating a range of raw scores (observed range). In some instances, the subject group comprises individuals with the same ancestry as the individual. Next, the individual raw score is compared to the observed range to calculate a percentage of risk relative to the subject population.

In another non-limiting example of calculating a GRS involving SNVs, an allelic odds ratio (OR) of each selected ancestry-specific SNV corresponding to an individual-specific SNV is provided and multiplied together. In some instances, the OR is obtained from a replicated, published, and/or peer reviewed GWAS. In some instances, an OR of each selected ancestry-specific SNV corresponding to an individual-specific SNV is provided. Next, the genotypic ORs for each ancestry-specific SNV are added together; the genotypic ORs for the individual are multiplied together. The genotypic ORs for the individual and the subject group are compared, and a percentile GRS is calculated.

In another non-limiting example of calculating a GRS involving an indel, a homozygous genotype for an insertion within the indel (II) is assigned a numerical value 2; a heterozygous genotype for an insertion within the indel (I) is assigned a numerical value 1; a genotype that is nonrisk (N) is assigned a numerical value 0. Next, each numerical value for all individual indels corresponding to an ancestry-specific indel, are added together, a divided by a total number of genetic variants used in the model, to generate a raw score for the individual (individual raw score). The same calculations are performed for each individual belonging to the subject group, thereby generating a range of raw scores (observed range). In some instances, the subject group comprises individuals with the same ancestry as the individual. Next, the individual raw score is compared to the observed range to calculate a risk percentile relative to the subject population.

In another non-limiting example of calculating a GRS involving indels, an odds ratio (OR) of each selected ancestry-specific indel corresponding to an individual-specific indel is provided and multiplied together. In some instances, the OR is obtained from a replicated, published, and/or peer reviewed GWAS. In some instances, an OR of each selected ancestry-specific indel corresponding to an individual-specific indel is provided and the ORs for each risk indel allele are multiplied to generate a genotypic OR for each subject in the subject group. Next, the same calculations are performed for the individual, to generate a genotypic OR for the individual. The genotypic ORs for the individual and the subject group are compared, and a percentile GRS is calculated.

In a non-limiting example of calculating a GRS involving CNVs, a genotype that is nonrisk (e.g., copy number is the same as wild-type, or a normal control) is assigned a numerical value 0, a genotype which comprises of 1 CNV is assigned a numerical value 1, a genotype which comprises of 2 CNVs is assigned a numerical value 2. Next, each numerical value for all individual CNVs corresponding to an ancestry-specific CNV, are added together, a divided by a total number of genetic variants used in the model, to generate a raw score for the individual (individual raw score). The same calculations are performed for each individual belonging to the subject group, thereby generating a range of raw scores (observed range). In some instances, the subject group comprises individuals with the same ancestry as the individual. Next, the individual raw score is compared to the observed range to calculate a risk percentile relative to the subject population.

In another non-limiting example of calculating a GRS involving CNVs, an odds ratio (OR) of each selected ancestry-specific CNV corresponding to an individual-specific CNV is provided and multiplied together. In some instances, the OR is obtained from a replicated, published, and/or peer reviewed GWAS. In some instances, an OR of each selected ancestry-specific CNV corresponding to an individual-specific CNV is provided and the ORs for each CNV are multiplied together to generate a genotypic OR for each subject in the subject group. Next, the same calculations are performed for the individual, to generate a genotypic OR for the individual. The genotypic ORs for the individual and the subject group are compared, and a percentile GRS is calculated.

Disclosed herein, in some embodiments, are methods, media, and systems for calculating a genetic risk score (GRS) using the methods disclosed above involving one or more SNVs and one or more CNVs, one or more SNVs and one or more indels, one or more CNVs and one or more indels, or one or more SNVs, one or more CNVs, and one or more indels.

### Phenotypic Traits

The majority of phenotypic traits and complex disease are the result of a combination of genetic and environmental factors, each of which increases or decreases susceptibility to developing the phenotypic trait. An ability to predict whether an individual has, or will develop, a phenotypic trait is useful for a variety of purposes, including, but not limited to, selecting a treatment regimen for the individual, proscribing a diet to the individual, recommending a product (e.g., skin care, hair care, cosmetics, supplements, vitamins, exercise, and the like).

The terms “phenotypic trait,” and “specific phenotypic trait” are used interchangeably herein to refer to an observable characteristic of an individual resulting from, at least, the genotype of the individual. The genetic risk prediction methods, media, and systems disclosed herein quantify the load of genetic variation in an individuals' genotype by analyzing the number and type of genetic variants, as compared to a reference population. The number and type of genetic variants present in a sample obtained from an individual can tell you whether the individual has an increased or decreased likelihood (or risk) of developing a certain phenotypic trait. In some cases, the specific phenotypic trait adversely affects the health or wellness of the individual. Disclosed herein, in some embodiments are methods, systems, and media for recommending behavioral change to prevent, mitigate, or ameliorate adverse effects of the specific phenotypic trait in an individual.

Aspects disclosed herein provide methods and systems of calculating a genetic risk score (GRS) representing the likelihood that an individual will develop a specific phenotypic trait. The GRS is based one or more genetic variants present in the genome of the individual, or genotype. In some embodiments, the one or more genetic variants is detected in a sample obtained from the individual using the methods disclosed herein. In some embodiments, the one or more genetic variants comprise a SNV, an indel, and/or a CNV. In some embodiments, the one or more genetic variants present in the genotype of the individual are associated with an increased likelihood that the individual has, or will develop, a specific phenotypic trait. In some embodiments, the one or more genetic variants present in the genotype of the individual are associated with a decreased likelihood that the individual has, or will develop, a specific phenotypic trait. In some embodiments, the phenotypic trait comprises a clinical trait, a subclinical trait, a physical exercise trait, a skin trait, a hair trait, an allergy trait, a nutrition trait, or a mental trait.

### Clinical and Subclinical Traits

In some embodiments, a clinical trait comprises a disease or condition, or subclinical trait of the disease or condition. In some embodiments, the clinical trait comprises a diagnosable disease or condition. In some embodiments, the subclinical trait comprises a sub-diagnosable disease, condition, or other phenotype associated with a disease or condition. In some embodiments, the disease or condition comprises a deficiency disease, a hereditary disease, or psychological disease. In some embodiments the disease or condition comprises an immunological disease and/or a metabolic disease cataract risk, glaucoma risk, joint inflammation risk, kidney stone risk, overall inflammation risk, pelvic floor dysfunction, inflammatory biomarker CRP, ESR, IL18, age-related cognitive decline, age-related hearing loss, vitiligo, elevated homocysteine risk. Non-limiting examples include insomnia risk, kidney stone risk, and periodontitis. In some embodiments, the immunological disease comprises autoimmune disease or disorders. Non-limiting examples of autoimmune diseases or disorders include Grave's disease, Hashimoto's thyroiditis, systemic lupus erythematosus (lupus), multiple sclerosis, rheumatoid arthritis, inflammatory bowel disease, Crohn's disease, ulcerative colitis, and cancer. Non-limiting examples of metabolic diseases or conditions include Type 1 diabetes, Type 2 diabetes, diseases affecting absorption of macronutrients (e.g., amino acids, carbohydrates, or lipids), diseases affecting absorption of micronutrients (e.g., vitamins or minerals), diseases affecting mitochondrial function, diseases affecting liver function (e.g., nonalcoholic fatty liver diseases), and diseases affecting kidney function. A subclinical trait may include a subdiagnosable condition or disorder associated with the disease or conditions disclosed herein.

### Skin Traits

In some embodiments, the phenotypic trait comprises a trait related to the skin of the individual (skin trait). In some embodiments, the skin trait comprises a rate of collagen breakdown. The rate of collagen breakdown may be affected by genetic variations within genes encoding MMP, MMP-3, MMP-1 collagen breakdown enzymes. Non-limiting examples of genetic variations within genes encoding collagen breakdown enzymes includes the single nucleotide variants (SNVs) disclosed in Table 1.

TABLE 1

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 1 | rs495366 | 11 | 102695108 | G | A | MMP | 0.64 | 6E−34 | 0.44 |
| 2 | rs11226373 | 11 | 104334239 | G | A | MMP-3, | 0.15 | 1E−18 | 0.44 |
| | | | | | | MMP-1 | | | |

In some embodiments, the skin trait comprises a level of dryness. Skin hydration, and therefore level of dryness, may be affected by genetic variations within the gene encoding aquaporin 3. A non-limiting example of a genetic variation within the gene encoding aquaporin 3 includes the SNV disclosed in Table 2.

TABLE 2

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 3 | rs17553719 | 9 | 33447579 | G | A | aquaporin 3 | 0.3 | NR | NR |

In some embodiments, the skin trait comprises an antioxidant deficiency of the skin. Antioxidant deficiency of the skin may be affected by genetic variations within genes encoding NQO1, SOD2, NFE2L2, GPX1, and/or CAT. Non-limiting examples of genetic variations within genes encoding NQO1, SOD2, NFE2L2, GPX1, and CAT includes the SNVs disclosed in Table 3.

TABLE 3

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 4 | rs1800566 | 16 | 69745145 | T | C | NAD(P)H | NR | NR | NR |
| | | | | | | dehydrogenase[quinone] 1 | | | |
| 5 | rs4880 | 6 | 160113872 | T | C | Superoxide dismutase II | NR | NR | NR |
| 6 | rs6706649 | 2 | 178130071 | T | C | Nuclear factor erythroid | NR | NR | NR |
| | | | | | | 2-related factor 2 | | | |
| 7 | rs6721961 | 2 | 178130037 | T | G | Nuclear factor erythroid | NR | NR | NR |
| | | | | | | 2-related factor 2 | | | |
| 8 | rs1050450 | 3 | 49394834 | C | T | Glutathione peroxidase 1 | NR | NR | NR |
| 9 | rs1001179 | 11 | 34460231 | G | A | Catalase | NR | NR | NR |

In some embodiments, the skin trait comprises an impairment to detoxify the skin. The skins ability to detoxify may be affected by genetic variations within genes encoding LOC157273, SGOL1, TB3C1D22B3, FST, MIR4432, RNASEH2C, and/or TGFB32. Non-limiting examples of genetic variations within genes encoding LOC157273, SGOL1, TB3C1D22B3, FST, MIR4432, RNASEH2C, and TGFB32 includes the SNVs disclosed in Table 4.

TABLE 4

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 10 | rs330071 | 8 | 9159895 | G | A | LOC157273 | 0.65 | 9E−07 | 0.21 |
| 11 | rs75430906 | 3 | 20717929 | A | G | SGOL1 | 0.00 | 1E−07 | 1.24 |
| 12 | rs149709 | 6 | 37278933 | C | T | TBC1D22B | 0.20 | 2E−06 | 0.17 |
| 13 | rs38055 | 5 | 52560644 | A | G | FST | 0.32 | 5E−09 | 0.17 |
| 14 | rs4671386 | 2 | 60514993 | C | A | MIR4432 | 0.43 | 2E−06 | 0.17 |
| 15 | rs478304 | 11 | 65494260 | T | G | RNASEH2C | 0.55 | 3E−11 | 0.18 |
| 16 | rs1159268 | 1 | 218844906 | A | G | TGFB2 | 0.35 | 4E−08 | 0.16 |

In some embodiments, the skin trait comprises skin glycation. Glycation may be affected by genetic variations within genes encoding SLC24A5, SLC45A2, BCN2, MC1R, C16orf55, SPATA33, ASIP, RALY, and/or NAT2. Non-limiting examples of genetic variations within genes encoding SLC24A5, SLC45A2, BCN2, MC1R, C16orf55, SPATA33, ASIP, RALY, and NAT2 include the SNVs disclosed in Table 5.

TABLE 5

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 17 | rs1834640 | 15 | 48392165 | G | A | SLC24A5 | 0.08 | 1E−50 | 2.53 |
| 18 | rs16891982 | 5 | 33951693 | C | G | SLC45A2 | 0.83 | 3E−11 | 1.58 |
| 19 | rs62543565 | 9 | 16901067 | A | C | BCN2 | 0.63 | 2E−07 | 0.15 |
| 20 | rs35063026 | 16 | 89736157 | T | C | MC1R, | 0.07 | 9E−15 | 0.33 |
| | | | | | | C16orf55, | | | |
| | | | | | | SPATA33 | | | |
| 21 | rs6059655 | 20 | 32665748 | A | G | ASIP, RALY | 0.08 | 3E−09 | 0.30 |
| 22 | rs4921914 | 8 | 18272438 | T | C | NAT2 | 0.81 | 8E−42 | 0.11 |

In some embodiments, the skin trait comprises pigmented spots. Pigmented spots of the skin may be affected by genetic variations in genes encoding SEC5L1, IRF4, MC1R, SLC45A2, TYR, NTM, ASIP, RALY. Non-limiting examples of genetic variations within genes encoding SEC5L1, IRF4, MC1R, SLC45A2, TYR, NTM, ASIP, RALY includes the SNVs disclosed in Table 6.

TABLE 6

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 23 | rs1805007 | 16 | 89986117 | T | C | MC1R | 0.05 | 1E−96 | 1.47 |
| 24 | rs12931267 | 16 | 89818732 | C | G | MC1R | 0.91 | 8E−23 | 0.44 |
| 25 | rs1540771 | 6 | 466033 | A | G | SEC5L1, | 0.42 | 4E−18 | 0.34 |
| | | | | | | IRF4 | | | |
| 26 | rs4268748 | 16 | 90026512 | T | C | MC1R | 0.72 | 3E−15 | 0.01 |
| 27 | rs16891982 | 5 | 33951693 | C | G | SLC45A2 | 0.83 | 3E−11 | 1.58 |
| 28 | rs1126809 | 11 | 89017961 | A | G | TYR | NR | 2E−08 | 0.60 |
| 29 | rs6059655 | 20 | 32665748 | G | A | ASIP, RALY | 0.90 | 1E−07 | 0.22 |

In some embodiments, the skin trait comprises youthfulness. “Youthfulness” as disclosed herein refers to a quality of the skin comprising a slow rate of aging, or appears newer or younger than it is. Youthfulness may be affected by genetic variations within genes encoding EDEM1. A non-limiting example of a genetic variation within the gene encoding EDEM1 includes the SNV disclosed in Table 7. In some embodiments, Youthfulness refers to a quality of the skin comprising a rate of aging that is slower by 1 month, 2 months, 3 months, 4 months, 5 months, 6 months, 7 months, 8 months, 9 months, 10 months, 11 months, 12 months, 1 year, 2 years 3 years, 4 years or 5 years, as compared to a rate of aging in an individual who does not express the SNV disclosed in Table 7.

TABLE 7

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 30 | rs7616661 | 3 | 5965543 | G | T | EDEM1 | 0.04 | 5E−08 | NR |

In some embodiments, the skin trait comprises photoaging. “Photoaging” as disclosed herein refers to the damage to the skin due to ultraviolet radiation and is a major contributor to premature aging. Photoaging may be affected by genetic variations within genes encoding MC1R, NTM, TYR, FBXO40, STXBP5L, ASIP, RALY, FANCA, ID4—RPL29P17. Non-limiting examples of genetic variations within genes encoding MC1R, NTM, TYR, FBXO40, STXBP5L, ASIP, RALY, FANCA, ID4—RPL29P17 include the SNVs disclosed in Table 8. PGP-28J′12

TABLE 8

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 31 | rs1805007 | 16 | 89986117 | T | C | MC1R | 0.14 | 2e−55 | 1.08 |
| 32 | rs12421680 | 11 | 131350968 | A | G | NTM | NR | 6e−06 | 0.41 |
| 33 | rs1126809 | 11 | 89017961 | A | G | TYR | NR | 2e−08 | 0.60 |
| 34 | rs322458 | 3 | 120585315 | G | A | FBXO40, | NR | 2e−08 | NR |
| | | | | | | STXBP5L | | | |
| 35 | rs6059655 | 20 | 32665748 | G | A | ASIP, RALY | 0.10 | 1e−07 | 0.22 |
| 36 | rs12931267 | 16 | 89818732 | C | G | FANCA | 0.91 | 8e−23 | 0.44 |
| 37 | rs9350204 | 6 | 19996808 | C | A | ID4 | 0.15 | 2e−06 | NR |
| | | | | | | RPL29P17 | | | |

In some embodiments, the skin trait comprises dermal sensitivity. “Dermal sensitivity” as disclosed herein refers to genetic variations that may cause skin barrier defects and promote skin sensitivity and irritation. Dermal sensitivity may be affected by genetic variations within genes encoding RNASEH2C, DDB32, C11orf49, SELL, TGFB32, SGOL1, ERI1, LOC157273, MFHAS1, MIR597, MIR4660, PPP1R31B, U6, TNKS, BC017578, T 3C1D22, AL833181, BCL11A, J153659, PAPOLG, MIR4432, Mir 562. Non-limiting examples of genetic variations within genes encoding RNASEH2C, DD12, C11orf49, SELL, TGFB32, SGOL1, ERI1, LOC157273, MFHAS1, MIR597, MIR4660, PPP1R31B, U6, TNKS, BC017578, TBC1D221B, AL833181, BCL11A, J1B153659, PAPOLG, MIR4432, Mir_562 include the SNVs disclosed in Table 9.

TABLE 9

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 38 | rs478304 | 11 | 65494260 | T | G | RNASEH2C | 0.55 | 3.00E−11 | 0.18 |
| 39 | rs747650 | 11 | 47176005 | G | A | DDB2 | 0.32 | 4.00E−09 | 0.22 |
| 40 | rs38055 | 5 | 52560644 | A | G | C11orf49 | 0.32 | 5.00E−09 | 0.17 |
| 41 | rs7531806 | 1 | 169651044 | A | G | SELL | 0.42 | 1.00E−08 | 0.20 |
| 42 | rs1159268 | 1 | 218844906 | A | G | TGFB2 | 0.35 | 4.00E−08 | 0.16 |
| 43 | rs75430906 | 3 | 20717929 | A | G | SGOL1 | 0.00 | 1.00E−07 | 1.24 |
| 44 | rs330071 | 8 | 9159895 | G | A | ERI1, LOC157273, | 0.65 | 9.00E−07 | 0.21 |
| | | | | | | MFHAS1, MIR597, | | | |
| | | | | | | MIR4660, PPP1R3B, | | | |
| | | | | | | U6, TNKS, BC017578 | | | |

In some embodiments, the skin trait comprises a sensitivity to the sun. Sensitivity to the sun refers to the predisposition of some skin types to damage as a result of moderate sun exposure. Sensitivity to the sun may be affected by genetic variations within genes encoding NTM, TYR, MC1R. Non-limiting examples of genetic variations within genes encoding NTM, TYR, MC1R include the SNV disclosed in Table 10.

TABLE 10

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 45 | rs12421680 | 11 | 131350968 | A | G | NTM | NR | 6.00E−06 | 0.41 |
| 46 | rs1126809 | 11 | 89017961 | A | G | TYR | NR | 2.00E−08 | 0.60 |
| 47 | rs1805007 | 16 | 89986117 | T | C | MC1R | NR | 2.00E−19 | 1.66 |

### Physical Exercise Trait

Disclosed herein, in some embodiments are physical exercise traits comprising a trait related to the fitness of the individual (fitness trait). In some embodiments, the fitness trait comprises exercise aversion. “Exercise aversion” refers to avoidance and/or or dislike of experience exercise. Exercise aversion may be affected by genetic variations within genes encoding PAPSS2, C18orf2, DNAPTP6, TMEM18, LEP, MC4R. Non-limiting examples of genetic variations within genes encoding PAPSS2, C18orf2, DNAPTP6, TMEM18, LEP, MC4R include the single nucleotide variants (SNVs) disclosed in Table 11.

TABLE 11

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 48 | rs10887741 | 10 | 89443310 | C | T | PAPSS2 | NR | 4E−06 | 0.28 |
| 49 | rs8097348 | 18 | 1595021 | A | G | C18orf2 | NR | 7E−06 | 0.31 |
| 50 | rs12612420 | 2 | 201158122 | G | A | DNAPTP6 | NR | 8E−06 | 0.36 |
| 51 | rs6548238 | 2 | 634905 | T | C | TMEM18 | 0.18 | 1E−02 | 11.80 |
| 52 | rs2167270 | 7 | 127881349 | A | G | LEP | NR | 2E−02 | NR |
| 53 | rs17782313 | 18 | 57851097 | C | T | MC4R | 0.79 | 2E−02 | 10.10 |

In some embodiments, the fitness trait comprises aerobic performance. Aerobic performance may e affected by genetic variations within genes encoding TSHR, ACSL1, PRDM1, DBX1, GRIN3A, ESRRB, ZIC4, CDH13. Non-limiting examples of genetic variations within genes TSHR, ACSL1, PRDM1, DBX1, GRIN3A, ESRRB, ZIC4, CDH13 include the SNVs disclosed in Table 12.

TABLE 12

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 54 | rs7144481 | 14 | 81610942 | C | T | TSHR | NR | 9E−08 | NR |
| 55 | rs6552828 | 4 | 185725416 | G | A | ACSL1 | NR | 1E−06 | NR |
| 56 | rs10499043 | 6 | 106247137 | A | G | PRDM1 | 0.13 | 4E−06 | NR |
| 57 | rs10500872 | 11 | 20245723 | A | G | DBX1 | NR | 6E−06 | NR |
| 58 | rs1535628 | 9 | 105016749 | G | A | GRIN3A | 0.09 | 7E−06 | NR |
| 59 | rs12893597 | 14 | 76812695 | T | C | ESRRB | NR | 7E−06 | NR |
| 60 | rs11715829 | 3 | 146957166 | A | G | ZIC4 | 0.08 | 9E−06 | NR |

In some embodiments, the fitness trait comprises difficulty losing weight. Difficulty losing weight may be affected by genetic variations within genes encoding FTO, TMEM18, MC4R, KCTD15, CHST8, PPARG, NEGR1, IRS1, SFRS10, ETV5, DGKG, ATP2A1, SH2B1, BDNF, SEC16B, RASAL2, NOS1AP, AIF1, NCR3, MSRA, TNKS, SPRY2, SH3PXD2B, NEURL1B, BCDIN3D, FAIM2, CHRNA9, RBM47, RGMA, MCTP2, MIR4275, PCDH7, TENM2, PRR16, FTMT, SLC24A5, SDCCAG8, COL25A1, NEURL1B, SH3PXD2B, ERBB4, MIR4776-2, STXBP6, NOVA1, DEFB1112, TFAP2D, EEF1A1P11—LOC105378866, MTIF3—RNU6-63P, NRXN3, CEP120, and/or LOC105378866—RN7SL831P. Non-limiting examples of genetic variations within genes encoding FTO, TMEM18, MC4R, KCTD15, CHST8, PPARG, NEGR1, IRS1, SFRS10, ETV5, DGKG, ATP2A1, SH2B1, BDNF, SEC16B, RASAL2, NOS1AP, AIF1, NCR3, MSRA, TNKS, SPRY2, SH3PXD2B, NEURL1B, BCDIN3D, FAIM2, CHRNA9, RBM47, RGMA, MCTP2, MIR4275, PCDH7, TENM2, PRR16, FTMT, SLC24A5, SDCCAG8, COL25A1, NEURL1B, SH3PXD2B, ERBB4, MIR4776-2, STXBP6, NOVA1, DEFB1112, TFAP2D, EEF1A1P11—LOC105378866, MTIF3—RNU6-63P, NRXN3, CEP120, and/or LOC105378866—RN7SL831P include SNVs disclosed in Table 13.

TABLE 13

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 61 | rs9939609 | 16 | 53820527 | A | T | FTO | 0.41 | 4E−51 | 0.33 |
| 62 | rs8050136 | 16 | 53816275 | A | C | FTO | 0.41 | 1E−47 | 8.04 |
| 63 | rs7561317 | 2 | 644953 | G | A | TMEM18 | 0.84 | 2E−18 | 6.47 |
| 64 | rs6499640 | 16 | 53769677 | A | G | FTO | 0.65 | 6E−14 | 5.50 |
| 65 | rs12970134 | 18 | 57884750 | A | G | MC4R | 0.30 | 5E−13 | 4.66 |
| 66 | rs9941349 | 16 | 53825488 | T | C | FTO | 0.43 | 6E−12 | 0.40 |
| 67 | rs29941 | 19 | 34309532 | C | T | KCTD15, CHST8 | 0.69 | 7E−12 | 4.18 |

In some embodiments, the fitness trait comprises endurance. Endurance may be affected by genetic variations within genes encoding PPARGC1A, PPAR-a, TSHR, ESRRB, and/or CDH13. Non-limiting examples of genetic variations within genes encoding PPARGC1A, PPAR-a, TSHR, ESRRB, and CDH13 include the SNVs disclosed in Table 14.

TABLE 14

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 68 | rs8192678 | 4 | 23815662 | G | A | PPARGC1A | 0.59 | 3E−03 | NR |
| 69 | rs4253778 | 22 | 46630634 | G | C | PPAR-a | 0.63 | 1E−03 | 0.81 |
| 70 | rs7144481 | 14 | 81610942 | C | T | TSHR | NR | 9E−08 | NR |
| 71 | rs12893597 | 14 | 76812695 | T | C | ESRRB | NR | 7E−06 | NR |
| 72 | rs9922134 | 16 | 83143453 | C | T | CDH13 | NR | 9E−06 | NR |

In some embodiments, the fitness trait comprises power. Power may be affected by genetic variations within genes encoding TSHR, ESRRB, and/or CDH13. Non-limiting examples of genetic variations within genes encoding TSHR, ESRRB, and CDH13 include SNVs disclosed in Table 15.

TABLE 15

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 73 | rs7144481 | 14 | 81610942 | T | C | TSHR | NR | 9E−08 | NR |
| 74 | rs12893597 | 14 | 76812695 | C | T | ESRRB | NR | 7E−06 | NR |
| 75 | rs9922134 | 16 | 83143453 | T | C | CDH13 | NR | 9E−06 | NR |

In some embodiments, the fitness trait comprises fitness benefits. “Fitness benefits” refers to individuals having certain genetic variations resulting in showing quicker and stronger benefits from exercise while others genetic variation may take longer and results are less apparent. Fitness benefits may be affected by genetic variations within genes encoding KLKB1, F12, CETP, APOE, APOC1, EDN1, SORT1, PLA2G7, LPL, LIPC, GALNT2, SCARB1, LIPG, MS4A4E, ABCA1, TMEM49, LOC101928635, MVK, MMAB, FLJ41733, FADS1, RREB1, COL8A1, and/or GCKR. Non-limiting examples of genetic variations within genes encoding KLKB1, F12, CETP, APOE, APOC1, EDN1, SORT1, PLA2G7, LPL, LIPC, GALNT2, SCARB1, LIPG, MS4A4E, ABCA1, TMEM49, LOC101928635, MVK, MMAB, FLJ41733, FADS1, RREB1, COL8A1, and GCKR include the SNVs disclosed in Table 16.

TABLE 16

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 76 | rs4253238 | 4 | 187148387 | T | C | KLKB1 | 0.54 | 1E−122 | 5.14 |
| 77 | rs2731672 | 5 | 176842474 | C | T | F12 | 0.76 | 1E−67 | 4.61 |
| 78 | rs1532624 | 16 | 57005479 | A | C | CETP | NR | 1E−66 | 3.09 |
| 79 | rs445925 | 19 | 45415640 | T | C | APOE, APOC1 | 0.89 | 1E−56 | 0.07 |
| 80 | rs1864163 | 16 | 56997233 | G | A | CETP | 0.80 | 7E−39 | 4.12 |
| 81 | rs9989419 | 16 | 56985139 | G | A | CETP | 0.65 | 3E−31 | 1.72 |
| 82 | rs5370 | 6 | 12296255 | G | T | EDN1 | 0.78 | 1E−27 | 2.96 |

In some embodiments, the fitness trait comprises reduced heart beat in response to exercise (e.g., recovery rate). Reduced heart beat in response to exercise may be affected by genetic variations within genes encoding RBPMS, PIWIL1, OR6N2, ERB1B4, CREB1, MAP2, and/or IKZF2. Non-limiting examples of genetic variations within genes encoding RBPMS, PIWIL1, OR6N2, ERBB14, CREB1, MAP2, and IKZF2 include the SNVs disclosed in Table 17.

TABLE 17

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 83 | rs2979481 | 8 | 30262786 | C | T | RBPMS | NR | NR | NR |
| 84 | rs11060842 | 12 | 130850356 | C | T | PIWIL1 | NR | NR | NR |
| 85 | rs857838 | 1 | 158750550 | A | C | OR6N2 | NR | NR | NR |
| 86 | rs10932380 | 2 | 212390350 | G | A | ERBB4 | NR | NR | NR |
| 87 | rs2254137 | 2 | 208444028 | A | C | CREB1 | NR | NR | NR |
| 88 | rs3768815 | 2 | 210552162 | T | C | MAP2 | NR | NR | NR |
| 89 | rs1394782 | 2 | 213200920 | G | A | ERBB4 | NR | NR | NR |

In some embodiments, the fitness trait comprises lean body mass. Lean body mass may be affected by genetic variations within genes encoding TRHR, DARC, GLYAT, FADS1, and/or FADS2. Non-limiting examples of genetic variations within genes encoding TRHR, DARC, GLYAT, FADS1, and FADS2 include the SNVs disclosed in Table 18.

TABLE 18

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 90 | rs7832552 | 8 | 110115676 | T | C | TRHR | 0.32 | 4E−10 | 0.06 |
| 91 | rs3027009 | 1 | 159173887 | A | G | DARC | NR | 7E−07 | NR |
| 92 | rs2507838 | 11 | 58472799 | A | C | GLYAT | 0.03 | 2E−08 | NR |
| 93 | rs174549 | 11 | 61571382 | G | A | FADS1, FADS2 | 0.30 | 8E−07 | 0.56 |

In some embodiments, the fitness trait comprises muscle soreness. Muscle soreness may be affected by genetic variations within genes encoding CD163L1, DARC, CD163, ABO, CRP, CD163, CADM3, CR1, NRNR, NINJ1, CFH, DARC, CPN1, CSF1, HBB, CCL2, and/or IGF2. Non-limiting examples of genetic variations within genes encoding CD163L1, DARC, CD163, ABO, CRP, CD163, CADM3, CR1, NRNR, NINJ1, CFH, DARC, CPN1, CSF1, HBB, CCL2, and IGF2 include the SNVs disclosed in Table 19.

TABLE 19

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 94 | rs4072797 | 12 | 7549009 | C | T | CD163L1 | 0.04 | 1E−88 | 0.24 |
| 95 | rs12075 | 1 | 159175354 | A | G | DARC | 0.49 | 4E−51 | 0.30 |
| 96 | rs117692263 | 12 | 7625014 | C | T | CD163 | 0.09 | 6E−28 | 0.09 |
| 97 | rs643434 | 9 | 136142355 | G | A | ABO | 0.26 | 9E−25 | 0.25 |
| 98 | rs7305678 | 12 | 7681181 | T | G | NR | 0.16 | 3E−21 | 0.07 |
| 99 | rs1341665 | 1 | 159691559 | G | A | CRP | 0.96 | 2E−20 | 0.20 |
| 100 | rs3026968 | 1 | 159147452 | T | C | CADM3 | 0.12 | 9E−14 | 0.24 |

In some embodiments, the fitness trait comprises muscle damage risk. “Muscle damage” refers to having a predisposition to increase muscle damage risk. Muscle damage risk may be affected by genetic variations within genes encoding IGF-II, MLCK, ACTN3, IL-6, and/or COL5A1. Non-limiting examples of genetic variations within genes encoding IGF-II, MLCK, ACTN3, IL-6, and COL5A1 include the SNVs disclosed in Table 20.

TABLE 20

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 101 | rs3213221 | 11 | 2157044 | G | C | IGF-II | 0.37 | 0.03 | NR |
| 102 | rs680 | 11 | 2153634 | A | G | IGF-II | 0.28 | 0.00 | NR |
| 103 | rs2700352 | 3 | 123550463 | T | C | MLCK | 0.20 | 0.02 | NR |
| 104 | rs1815739 | 11 | 66328095 | C | T | ACTN3 | 0.48 | 0.03 | NR |
| 105 | rs1800795 | 7 | 22766645 | C | G | IL-6 | 0.20 | 0.01 | 1.19 |
| 106 | rs12722 | 9 | 137734416 | T | C | COL5A1 | 0.61 | 0.01 | 0.60 |

In some embodiments, the fitness trait comprises muscle repair impairment. Muscle repair impairment may be affected by genetic variations within genes encoding HCP5, HCG26, MICB, ATP6V1G2, and/or DDX39B3. Non-limiting examples of genetic variations within genes encoding HCP5, HCG26, MICB, ATP6V1G2, and DDX39B include the SNVs disclosed in Table 21.

TABLE 21

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 107 | rs115902351 | 6 | 31434621 | G | A | HCP5, HCG26 | NR | 2E−45 | 0.81 |
| 108 | rs3130614 | 6 | 31476458 | A | T | MICB | NR | 4E−48 | 0.84 |
| 109 | rs9267488 | 6 | 31514247 | G | A | ATP6V1G2, | NR | 6E−49 | 0.84 |
| | | | | | | DDX39B | | | |

In some embodiments, the fitness trait comprises a stress fracture risk. A stress fracture risk may be affected by genetic variations within genes encoding LOC101060363—LOC105376856, ZBT1B40, EN1, FLJ42280, COLEC10, WNT16, ESR1, ATP6V1G1, CLDN14, ESR1FABP3P2, ADAMTS8, SOST, CLDN14, MEF2C, KCNH1, C6orf, CKAP5, C17orf53, SOST, TNFRSF11A, LCO5373519—LOC728815, PTCH1, SMOC1, LOC646794—LOC101928765, and/or LOC105377045—MRPS31P1. Non-limiting examples of genetic variations within genes encoding LOC101060363—LOC105376856, ZBT1B40, EN1, FLJ42280, COLEC10, WNT16, ESR1, ATP6V1G1, CLDN14, ESR1FABP3P2, ADAMTS18, SOST, CLDN14, MEF2C, KCNH1, C6orf97, CKAP5, C17orf53, SOST, TNFRSF11A, LOC105373519—LOC728815, PTCH1, SMOC1, LOC646794—LOC101928765, and LOC105377045—MRPS31P1 include the SNVs disclosed in Table 22.

TABLE 22

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 110 | rs7524102 | 1 | 22698447 | A | G | LOC101060363 | 0.18 | 1E−16 | 0.15 |
| | | | | | | LOC105376856 | | | |
| 111 | rs115242848 | 2 | 119507607 | C | T | EN1 | 0.99 | 8E−13 | 0.35 |
| 112 | rs10429035 | 7 | 96119481 | G | A | FLJ42280 | NR | 4E−12 | NR |
| 113 | rs6993813 | 8 | 120052238 | C | T | COLEC10 | 0.50 | 3E−11 | 0.09 |
| 114 | rs10242100 | 7 | 120983343 | A | G | WNT16 | NR | 2E−10 | NR |
| 115 | rs1038304 | 6 | 151933175 | G | A | ESR1 | 0.53 | 4E−10 | 0.08 |
| 116 | rs10817638 | 9 | 117322542 | A | G | ATP6V1G1 | 0.65 | 3E−09 | 0.22 |

In some embodiments, the fitness trait comprises overall injury risk. Overall injury risk may be affected by genetic variations within genes encoding HAO1, RSPO2, EMC2, EIF3E, CCDC91, PTHLH, LOC100506393, LINC00536, EIF3H, CDC5L, SUPT3H, and/or MIR4642. Non-limiting examples of genetic variations within genes encoding HAO1, RSPO2, EMC2, EIF3E, CCDC91, PTHLH, LOC100506393, LINC00536, EIF3H, CDC5L, SUPT3H, and MIR4642 include the SNVs disclosed in Table 23.

TABLE 23

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 117 | rs2423294 | 20 | 7819768 | T | C | HAO1 | 0.16 | 1E−13 | 0.34 |
| 118 | rs374810 | 8 | 109096029 | G | A | RSPO2, EMC2, | 0.61 | 2E−13 | 0.29 |
| | | | | | | EIF3E | | | |
| 119 | rs1979679 | 12 | 28406515 | T | C | CCDC91, | 0.36 | 4E−12 | 0.26 |
| | | | | | | PTHLH | | | |
| 120 | rs11045000 | 12 | 20184146 | A | G | LOC100506393 | 0.46 | 3E−11 | 0.25 |
| 121 | rs13279799 | 8 | 117541607 | G | A | LINC00536, | 0.32 | 1E−10 | 0.25 |
| | | | | | | EIF3H | | | |
| 122 | rs927485 | 6 | 44538139 | C | T | CDC5L, | 0.14 | 9E−09 | 0.29 |
| | | | | | | SUPT3H, | | | |
| | | | | | | MIR4642 | | | |

In some embodiments, the fitness trait comprises resting metabolic heart rate impairment. Resting metabolic heart rate impairment may be affected by genetic variations within genes encoding FTO. A non-limiting example of genetic variation within genes encoding FTO includes the SNV disclosed in Table 24.

TABLE 24

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 123 | rs17817449 | 16 | 53813367 | T | G | FTO | 0.61 | 0.04 | NR |

### Nutritional Trait

Disclosed herein, in some embodiments, is a nutritional trait comprising a vitamin deficiency, a mineral deficiency, an antioxidant deficiency, a metabolic imbalance, a metabolic impairment, a metabolic sensitivity, an allergy, satiety, and/or the effectiveness of a healthy diet.

In some embodiments, the nutritional trait comprises a vitamin deficiency. In some instances, the vitamin deficiency comprises a deficiency in Vitamin A, Vitamin B1, Vitamin B2, Vitamin B3, Vitamin B5, Vitamin B6, Vitamin B7, Vitamin B8, Vitamin B9, Vitamin B12, Vitamin C, Vitamin D, Vitamin E, or Vitamin K. A vitamin deficiency may be affected by genetic variations within genes encoding GC, FUT2, HAAO, BCMO1, ALPL, CYP2R1, MS4A3, FFAR4, TTR, CUBN, FUT6, ZNF259, LOC100128347, APOA5, SIK3, BUD13, ZNF259, APOA5, BUD13, KYNU, NBPF3, TCN1, CYP4F2, PDE3B, CYP2R1, CALCA, CALCP, OR7E41P, APOA5, CLYBL, NADSYN1, DHCR7, SCARB1, RNU7-49P, COPB1, RRAS2, PSMA1, PRELID2, CYP2R1, PDE3B, CALCA, CALCP, OR7E41P, MUT, ZNF259, CTNAA2, CDO1, SLC23A1, KCNK9, CYP4F2, LOC729645, ZNF259, BUD13, ST6GALNAC3, NKAIN3, VDAC1P12, RASIP1, MYT1L, PAX3, NPY, ADCYAP1R1, HSF5, RNF43, MTMR4, TMEM215-ASS1P12, FAM155A, CD44, BRAF, CD4, LEPREL2, GNB3, MKLN1, SLC6A1, PRICKLE2, SVCT1, and/or SVCT2. Non-limiting examples of genetic variations within genes encoding GC, FUT2, HAAO, BCMO1, ALPL, CYP2R1, MS4A3, FFAR4, TTR, CUBN, FUT6, ZNF259, LOC100128347, APOA5, SIK3, BUD13, ZNF259, APOA5, BUD13, KYNU, NBPF3, TCN1, CYP4F2, PDE3B, CYP2R1, CALCA, CALCP, OR7E41P, APOA5, CLYBL, NADSYN1, DHCR7, SCARB1, RNU7-49P, COPB1, RRAS2, PSMA1, PRELID2, CYP2R1, PDE3B, CALCA, CALCP, OR7E41P, MUT, ZNF259, CTNAA2, CDO1, SLC23A1, KCNK9, CYP4F2, LOC729645, ZNF259, BUD13, ST6GALNAC3, NKAIN3, VDAC1P12, RASIP1, MYT1L, PAX3, NPY, ADCYAP1R1, HSF5, RNF43, MTMR4, TMEM215—ASS1P12, FAM155A, CD44, BRAF, CD4, LEPREL2, GNB3, MKLN1, SLC6A1, PRICKLE2, SVCT1, and SVCT2 include the SNVs listed in Table 25.

TABLE 25

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 124 | rs7041 | 4 | 72618334 | T | G | GC | 0.35 | 1E−246 | 2109.34 |
| 125 | rs705117 | 4 | 72608115 | G | A | GC | 0.13 | 5E−91 | 2026.78 |
| 126 | rs2282679 | 4 | 72608383 | C | A | GC | 0.26 | 2E−49 | 0.38 |
| 127 | rs1047781 | 19 | 49206631 | A | T | FUT2 | NR | 4E−36 | 70.21 |
| 128 | rs4953657 | 2 | 42993782 | T | C | HAAO | 0.39 | 2E−32 | 0.42 |
| 129 | rs6564851 | 16 | 81264597 | T | G | BCMO1 | 0.61 | 2E−24 | 0.15 |
| 130 | rs602662 | 19 | 49206985 | G | A | FUT2 | 0.53 | 3E−20 | 49.77 |

In some embodiments, the nutritional trait comprises a mineral deficiency. In some instances, the mineral deficiency comprises a deficiency in calcium, iron, magnesium, zinc, and/or selenium. In some instances, the mineral deficiency may be affected by genetic variations within genes encoding CASR, TF, TFR2, SCAMP5, PPCDC, ARSB, BHMT2, DMGDH, ATP2B1, DCDC5, TRPM6, SHROOM3, CYP24A1, BHMT, BHMT2, JMY, TMPRSS6, GCKR, KIAA0564, DGKH, HFE, GATA3, VKORCIL1, MDS1, MUC1, CSTA, JMY, HOMER1, MAX, FNTB, SLC36A4, CCDC67, MIR379, FGFR2, LUZP2, PAPSS2, HOXD9, LOC102724653—IGLV4-60, HOOK3, FNTA, MEOX2, LOC101928964, PRPF8, MGC14376, SMYD4, SERPINF2, SERPINF1, WDR81, MIR4778, MEIS1-AS3, PRDM9, CALCOCO1, HOXC13, GPR39, SLC22A16, CDK19, TMOD1, TXNRD1, NFYB, MYOM2, CSMD1, KBTBD11, ARHGEF10, DYNC2H1, DCUN1D5, PDGFD, PRMT7, SERPINF2, WDR81, CRMP1, FLJ46481, KHDRBS2—LOC100132056, CD109, LOC100616530, SLC16A7, FLRT2, KYNU, ARHGAP15, RARB, C3orf58, PLOD2, RPRM, GALNT13, EPHA6, RGS14, SLC34A1, SLC22A18, PHLDA2, CDKN1C, NAP1L4, LOC101929578, ZNF14, ZNF101, ATP13A1, PYGB, CHD5, SDCCAG8, XDH, SRD5A2, CMYA5, RP11-314C16.1, TFAP2A, PTPRN2, CA1, KNOP1P1, RNU7-14P—LOC107987283, FNDC4, IFT172, GCKR, C2orf16, CBLB, LINC00882, LOC107983965, MIR4790, AC069277.1, IRX2, C5orf38, ZNF521, SS18, ATG4C, LPHN2, TTLL7, SAG, DGKD, RN7SKP61—MRPS17P3, GPBP1, STXBP6, NOVA1, TMEM211, and/or MT2A. Non-limiting examples of genetic variations within genes encoding CASR, TF, TFR2, SCAMP5, PPCDC, ARSB, BHMT2, DMGDH, ATP2B1, DCDC15, TRPM6, SHROOM3, CYP24A1, BHMT, BHMT2, JMY, TMPRSS6, GCKR, KIAA0564, DGKH, HFE, GATA3, VKORC1L1, MDS1, MUC1, CSTA, JMY, HOMER1, MAX, FNTB, SLC36A4, CCDC167, MIR379, FGFR2, LUZP2, PAPSS2, HOXD9, LOC102724653—IGLV4-60, HOOK3, FNTA, MEOX2, LOC101928964, PRPF8, MGC14376, SMYD4, SERPINF2, SERPINF1, WDR81, MIR4778, MEIS1-AS3, PRDM9, CALCOCO1, HOXC13, GPR39, SLC22A16, CDK19, TMOD1, TXNRD1, NFYB, MYOM2, CSMD1, KBTBD11, ARHGEF10, DYNC2H1, DCUN1D5, PDGFD, PRMT7, SERPINF2, WDR81, CRMP1, FLJ46481, KHDRBS2—LOC100132056, CD109, LOC100616530, SLC16A7, FLRT2, KYNU, ARHGAP15, RARB, C3orf58, PLOD2, RPRM, GALNT13, EPHA6, RGS14, SLC34A1, SLC22A18, PHLDA2, CDKN1C, NAP1L4, LOC101929578, ZNF14, ZNF101, ATP13A1, PYGB, CHD5, SDCCAG8, XDH, SRD5A2, CMYA5, RP11-314C16.1, TFAP2A, PTPRN2, CA1, KNOP1P1, RNU7-14P—LOC107987283, FNDC4, IFT172, GCKR, C2orf16, CBLB, LINC00882, LOC107983965, MIR4790, AC069277.1, IRX2, C5orf38, ZNF521, SS18, ATG4C, LPHN2, TTLL7, SAG, DGKD, RN7SKP61—MRPS17P3, GPBP1, STXBP6, NOVA1, TMEM211, and MT2A include the SNVs listed in Table 26.

TABLE 26

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 131 | rs1801725 | 3 | 122003757 | G | T | CASR | 0.15 | 9E−86 | 0.07 |
| 132 | rs8177240 | 3 | 133477701 | T | G | TF | 0.67 | 7E−20 | 0.07 |
| 133 | rs7385804 | 7 | 100235970 | C | A | TFR2 | 0.38 | 1E−18 | 0.06 |
| 134 | rs2120019 | 15 | 75334184 | C | T | SCAMP5, | NR | 2E−18 | 0.29 |
| | | | | | | PPCDC | | | |
| 135 | rs17823744 | 5 | 78344976 | A | G | ARSB, | 0.12 | 1E−16 | 0.05 |
| | | | | | | BHMT2, | | | |
| | | | | | | DMGDH | | | |
| 136 | rs7965584 | 12 | 90305779 | G | A | ATP2B1 | 0.29 | 1E−16 | 0.01 |
| 137 | rs3925584 | 11 | 30760335 | C | T | DCDC5 | 0.45 | 5E−16 | 0.01 |

In some embodiments, the nutritional trait comprises an antioxidant deficiency. In some instances, the antioxidant deficiency comprises a deficiency in glutathione, and/or coenzyme Q10 (CoQ10). The antioxidant deficiency may be affected by genetic variations within genes encoding GGT1, GGTLC2, MYL2, C12orf27, HNF1A, OAS1, C14orf73, ZNF827, RORA, EPHA2, RSG1, MICAL3, DPM3, EFNA1, PKLR, GCKR, C2orf16, NEDD4L, MYO1B, STAT4, CCBL2, PKN2, SLC2A2, ITGA1, DLG5, FUT2, ATP8B1, EFHD1, CDH6, CD276, FLJ37644, SOX9, DDT, DDTL, GSTT1, GSTT2B, MIF, MLIP, MLXIPL, DYNLRB2, CEPT1, DENND2D, COLEC12, LOC101927479—ARHGEF19, LOC105377979, MMP26, DNM1, LUZP1, ADH5P2—LOC553139, FST, MIR4708—LOC105370537, LOC105373450—KCNS3, LOC107984041-GRIK2, LINC01520, and/or NQO1. Non-limiting examples of genetic variations within genes encoding GGT1, GGTLC2, MYL2, C12orf27, HNF1A, OAS1, C14orf73, ZNF827, RORA, EPHA2, RSG1, MICAL3, DPM3, EFNA1, PKLR, GCKR, C2orf16, NEDD4L, MYO1B, STAT4, CCBL2, PKN2, SLC2A2, ITGA1, DLG5, FUT2, ATP8B1, EFHD1, CDH6, CD276, FLJ37644, SOX9, DDT, DDTL, GSTT1, GSTT2B, MIF, MLIP, MLXIPL, DYNLRB2, CEPT1, DENND2D, COLEC12, LOC101927479—ARHGEF19, LOC105377979, MMP26, DNM1, LUZP1, ADH5P2—LOC553139, FST, MIR4708—LOC105370537, LOC105373450—KCNS3, LOC107984041—GRIK2, LINC01520, and NQO1 include the SNVs listed in Table 27.

TABLE 27

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 138 | rs2073398 | 22 | 24999104 | C | G | GGT1, GGTLC2 | 0.66 | 1E−109 | 12.30 |
| 139 | rs12229654 | 12 | 111414461 | T | G | MYL2 | 0.86 | 9E−58 | 0.01 |
| 140 | rs7310409 | 12 | 121424861 | A | G | C12orf27, HNF1A | 0.41 | 7E−45 | 6.80 |
| 141 | rs11066453 | 12 | 113365621 | A | G | OAS1 | 0.87 | 6E−44 | 0.01 |
| 142 | rs944002 | 14 | 103572815 | A | G | C14orf73 | 0.79 | 6E−29 | 6.30 |
| 143 | rs4547811 | 4 | 146794621 | T | C | ZNF827 | 0.82 | 3E−27 | 6.40 |
| 144 | rs339969 | 15 | 60883281 | C | A | RORA | 0.38 | 7E−20 | 4.50 |

In some embodiments, the nutritional trait comprises a metabolic imbalance. In some instances, the metabolic imbalance comprises a glucose imbalance. A metabolic imbalance may be affected by genetic variations within genes encoding G6PC2, MTNR1B, GCK, ADCY5, MADD, ADRA2A, GCKR, MRPL33, ABCB11, FADS1, PCSK1, CRY2, ARAP1, SIX2, SIX3, PPP1R31B, SMCA2, GLIS3, DPYSL5, SLC30A8, PROX1, CDKN2A, CDKN2B, FOXA2, TMEM195, DGKB, PDK1, RAPGEF4, PDX1, CDKAL1, KANK1, IGF1R, C2CD4B, LEPR, GRB10, LMO1, RREB1, FBXL10, and/or FOXN3. Non-limiting examples of genetic variations within genes encoding G6PC2, MTNR1B, GCK, ADCY5, MADD, ADRA2A, GCKR, MRPL33, ABCB11, FADS1, PCSK1, CRY2, ARAP1, SIX2, SIX3, PPP1R3B, SMCA2, GLIS3, DPYSL5, SLC30A8, PROX1, CDKN2A, CDKN2B, FOXA2, TMEM195, DGKB, PDK1, RAPGEF4, PDX1, CDKAL1, KANK1, IGF1R, C2CD4B, LEPR, GRB10, LMO1, RREB1, FBXL10, and FOXN3 include the SNVs listed in Table 28.

TABLE 28

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 145 | rs560887 | 2 | 169763148 | C | T | G6PC2 | 0.70 | 9E−218 | 0.08 |
| 146 | rs10830963 | 11 | 92708710 | G | C | MTNR1B | 0.30 | 6E−175 | 0.07 |
| 147 | rs4607517 | 7 | 44235668 | A | G | GCK | 0.16 | 7E−92 | 0.06 |
| 148 | rs11708067 | 3 | 123065778 | A | G | ADCY5 | 0.78 | 7E−22 | 0.03 |
| 149 | rs7944584 | 11 | 47336320 | A | T | MADD | 0.75 | 2E−18 | 0.02 |
| 150 | rs10885122 | 10 | 113042093 | G | T | ADRA2A | 0.87 | 3E−16 | 0.02 |
| 151 | rs3736594 | 2 | 27995781 | C | A | MRPL33 | 0.73 | 1E−15 | 0.00 |

In some embodiments, the nutritional trait comprises a metabolic impairment. In some instances, the metabolic impairment comprises impaired metabolism of caffeine and/or a drug. A metabolic impairment may be affected by genetic variations within genes encoding MTNR1B, CACNA2D3, NEDD4L, AC105008.1, P2RY2, RP11-479A21.1, MTUS2, PRIMA1, and/or RP11-430J3.1. Non-limiting examples of genetic variations within genes encoding MTNR1B, CACNA2D3, NEDD4L, AC105008.1, P2RY2, RP11-479A21.1, MTUS2, PRIMAL and RP11-430J3.1 include the SNVs listed in Table 29.

TABLE 29

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 152 | rs10830964 | 11 | 92719681 | C | T | MTNR1B | 0.88 | 5E−06 | 0.48 |
| 153 | rs11706236 | 3 | 55188273 | A | G | CACNA2D3 | 0.86 | 4E−06 | 0.46 |
| 154 | rs158856 | 18 | 55910523 | C | T | NEDD4L | 0.66 | 7E−06 | 0.34 |
| 155 | rs16905439 | 8 | 136989204 | C | T | AC105008.1 | 0.99 | 9E−06 | 1.20 |
| 156 | rs1791933 | 11 | 72894848 | C | T | P2RY2 | 0.98 | 8E−06 | 1.31 |
| 157 | rs2065779 | 10 | 112877801 | G | C | RP11-479A21.1 | 0.93 | 3E−06 | 0.60 |
| 158 | rs2388082 | 13 | 29961332 | C | G | MTUS2 | 0.89 | 4E−06 | 0.52 |

In some embodiments, the nutritional trait comprises a metabolic sensitivity. In some instances, the metabolic sensitivity comprises gluten sensitivity, sensitive to salt, glycan sensitivity, and/or lactose sensitivity. A metabolic sensitivity may be affected by genetic variations within genes encoding PIBF1, IRAK1BP1, PRMT6, CDCA7, NOTCH4, HLA-DRA, BTNL2, ARSJ, CSMD1, ALX4, NSUN3, RAB39BP1, GPR65, C15orf32, TSN, CREB1, and/or ARMC9. Non-limiting examples of genetic variations within genes encoding PIBF311, IRAK1BP1, PRMT6, CDCA7, NOTCH4, HLA-DRA, BTNL2, ARSJ, CSMD1, ALX4, NSUN3, RAB39BP1, GPR65, C15orf32, TSN, CREB1, and ARMC9 include the SNVs listed in Table 30.

TABLE 30

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 159 | rs8002688 | 13 | 73559982 | T | C | PIBF1 | 0.04 | 2E−09 | 2.04 |
| 160 | rs16890334 | 6 | 79556166 | T | C | IRAK1BP1 | 0.94 | 4E−09 | 5.43 |
| 161 | rs1330225 | 1 | 106835943 | T | C | PRMT6 | 0.99 | 7E−09 | 5.16 |
| 162 | rs10930597 | 2 | 174326845 | C | T | CDCA7 | 0.95 | 4E−08 | 3.37 |
| 163 | rs3135350 | 6 | 32392981 | G | A | NOTCH4, | 0.05 | 9E−08 | 0.51 |
| | | | | | | HLA-DRA, | | | |
| | | | | | | BTNL2 | | | |
| 164 | rs7658266 | 4 | 114863706 | C | T | ARSJ | 0.79 | 3E−07 | 2.35 |
| 165 | rs2627282 | 8 | 2780956 | G | A | CSMD1 | 0.98 | 3E−07 | 2.33 |

In some embodiments, the nutritional trait comprises a food allergy. In some embodiments, the food allergy comprises a peanut allergy. An allergy to peanut may be affected by genetic variations within genes encoding HLA-DRB1, HLA-DQA1, HLA-DQB1, HLA-DQA2, HCG27, HLA-C, ADGB, RPS15P9, MUM1, RYR1, LIN4C00992, L0C100129526, FAM118A, SMC1B, MIATNB, ATP2C2, PLAGL1, MRPL42, and/or STAT6. Non-limiting examples of genetic variations within genes encoding HLA-DR1, HLA-DQA1, HLA-DQB1, HLA-DQA2, HCG27, HLA-C, ADGB, RPS15P9, MUM1, RYR1, LINC00992, LOC100129526, FAM118A, SMC1B, MIATNB, ATP2C2, PLAGL1, MRPL42, and STAT6 include the SNVs listed in Table 31.

TABLE 31

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 173 | rs9275596 | 6 | 32681631 | C | T | HLA-DRB1, HLA-DQA1, | 0.36 | 6E−11 | 0.53 |
| | | | | | | HLA-DQB1, HLA-DQA2 | | | |
| 174 | rs3130941 | 6 | 31197514 | C | G | HCG27, HLA-C | 0.25 | 1E−10 | 0.10 |
| 175 | rs4896888 | 6 | 147098991 | C | T | ADGB | 0.56 | 3E−07 | 1.90 |
| 176 | rs758147 | 19 | 1322312 | C | T | RPS15P9, MUM1 | 0.62 | 1E−06 | 1.83 |
| 177 | rs3786829 | 19 | 39014184 | C | T | RYR1 | 0.16 | 2E−06 | 1.99 |
| 178 | rs1830169 | 5 | 117048725 | C | T | LINC00992, | 0.21 | 4E−06 | 0.77 |
| | | | | | | LOC100129526 | | | |
| 179 | rs998706 | 22 | 45735606 | T | C | FAM118A, SMC1B | 0.54 | 4E−06 | 0.60 |

In some embodiments, the nutritional trait comprises satiety. Satiety may be affected by genetic variations within genes encoding LEPR. Non-limiting examples of genetic variations within genes encoding LEPR include the SNVs listed in Table 32.

TABLE 32

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 187 | rs4655555 | 1 | 66080269 | A | T | LEPR | 0.22 | 2.0E−08 | 0.07 |
| 188 | rs12062820 | 1 | 65970495 | T | C | LEPR | NR | 1.6E−14 | 0.10 |

Effectiveness of a healthy diet may be affected by genetic variations within genes encoding FGF21, ZPR1, TANK, FNBP1, RNU6-229P—LOC105375346, ARGFX, BEND3, SUMO26—LOC105377740, LOC101929216—GDF10, LOC105377451—LOC105377622, CPA3, KCNQ3, THBS4, TENM2, HSPA91P2—LOC105372045, LINC00113—LINC00314, SH31BGRL2, NKAIN2, OPRM1, LOC105377795, NCALD, L9C728503, LOC105370491, LOC107985318—MIA3, BECNP2—LYPLA1P3, LOC105376778—LINC01082, SOX5, LHX5-AS1—LOC105369990, NBAS, ABCG2, PPARγ2, CLOCK, RARB, FTO, IRS1, TCF7L2, HNMT, and/or PFKL. Non-limiting examples of genetic variations within genes encoding FGF21, ZPR1, TANK, FNBP1, RNU6-229P—LOC105375346, ARGFX, BEND3, SUMO21P6—LOC105377740, LOC101929216—GDF10, LOC105377451—LOC105377622, CPA3, KCNQ3, THBS4, TENM2, HSPA91P2—LOC105372045, LINC00113—LINC00314, SH3BGRL2, NKAIN2, OPRM1, LOC105377795, NCALD, LOC728503, LOC105370491, LOC7985318—MIA3, BECN1P2—LYPL1P3, LOC105376778—LIN4C01082, SOX5, LHX5-AS1—LOC105369990, NBAS, ABCG2, PPARγ2, CLOCK, RARB, FTO, IRS1, TCF7L2, HNMT, and PFKL include the SNVs listed in Table 33.

TABLE 33

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 189 | rs838145 | 19 | 49248730 | G | A | FGF21 | 0.54 | 4E−10 | 0.22 |
| 190 | rs964184 | 11 | 116648917 | C | G | ZPR1 | 0.17 | 1E−09 | 0.30 |
| 191 | rs197273 | 2 | 161894663 | G | A | TANK | 0.52 | 1E−07 | 0.23 |
| 192 | rs2007126 | 9 | 132684007 | A | G | FNBP1 | 0.16 | 2E−07 | 0.05 |
| 193 | rs6959964 | 7 | 68905738 | T | C | RNU6-229P - | 0.63 | 3E−07 | 0.26 |
| | | | | | | LOC105375346 | | | |
| 194 | rs13096657 | 3 | 121300728 | T | C | ARGFX | 0.14 | 4E−07 | 0.37 |
| 195 | rs3749872 | 6 | 107388504 | T | C | BEND3 | 0.95 | 4E−07 | 0.59 |

### Allergy Trait

Disclosed herein, in some embodiments, are allergy traits. In some embodiments, an allergy trait comprises a skin allergy, a dust allergy, an insect sting allergy, a pet allergy, an eye allergy, a drug allergy, a latex allergy, a mold allergy, and/or a pest allergy. In some embodiments, the allergy trait comprises allergic inflammation. “Allergic inflammation,” as used herein refers to inflammation caused by, or associated with, an allergic reaction.

In some embodiments, the nutritional trait comprises allergic inflammation. In some instances, allergic inflammation may be affected by genetic variations within genes encoding FCER1A, LRRC32, C11orf30, IL13, OR10J3, HLA-A, STAT6, TSLP, SLC25A46, WDR36, CAMK4, HLA-DQB1, HLA-DQA1, STAT6, NAB2, DARC, IL18R1, IL1RL1, IL18RAP, FAM114A1, MIR574, TLR10, TLR1, TLR6, LPP, BCL6, MYC, PVT1, IL2, ADAD1, KIAA1109, IL21, HLA region, TMEM232, SLCA25A46, HLA-DQA2, HLA-G, MICA, HLA-C, HLA-B, MICB, HLA-DRB1, IL4R, ID2, LOC730217, OPRK1, WWP2, EPS15, ANAPC1, LPP, LOC101927026, IL4R, IL21R, SUCLG2, TMEM108, DNAH5, OR6X1, DOCK10, ABL2, COL21A1, and/or CDH13. Non-limiting examples of genetic variations within genes encoding FCER1A, LRRC32, C11orf30, IL13, OR10J3, HLA-A, STAT6, TSLP, SLC25A46, WDR36, CAMK4, HLA-DQB1, HLA-DQA1, STAT6, NAB2, DARC, IL18R1, IL1RL1, IL18RAP, FAM114A1, MIR574, TLR10, TLR1, TLR6, LPP, BCL6, MYC, PVT1, IL2, ADAD1, KIAA1109, IL21, HLA region, TMEM232, SLCA25A46, HLA-DQA2, HLA-G, MICA, HLA-C, HLA-B, MICB, HLA-DRB1, IL4R, ID2, LOC730217, OPRK1, WWP2, EPS15, ANAPC1, LPP, LOC101927026, IL4R, IL21R, SUCLG2, TMEM108, DNAH5, OR6X1, DOCK10, ABL2, COL21A1, and CDH13 include the SNVs listed in Table 34.

TABLE 34

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 166 | rs2251746 | 1 | 159272060 | T | C | FCER1A | 0.74 | 5E−26 | 0.09 |
| 167 | rs2155219 | 11 | 76299194 | T | G | LRRC32, C11orf30 | 0.47 | 1E−18 | 0.17 |
| 168 | rs20541 | 5 | 131995964 | A | G | IL13 | 0.19 | 3E−18 | 0.08 |
| 169 | rs4656784 | 1 | 159326880 | A | G | OR10J3 | 0.80 | 2E−16 | 0.08 |
| 170 | rs2571391 | 6 | 29923838 | A | C | HLA-A | 0.68 | 1E−15 | 0.06 |
| 171 | rs1059513 | 12 | 57489709 | T | C | STAT6 | 0.90 | 1E−14 | 0.26 |
| 172 | rs10056340 | 5 | 110190052 | G | T | TSLP, SLC25A46, | 0.17 | 5E−14 | 0.18 |
| | | | | | | WDR36, CAMK4 | | | |

In some embodiments, the allergy trait comprises a pest allergy. In some embodiments, the pest allergy comprises an allergy to mites. An allergy to mites may be affected by genetic variations within genes encoding LOC730217, OPRK1, OR6X1, DOCK10, CDH13, Cap S, IL4, ADAM33, IRS2, ABHD13, LINC00299, IL18, CYP2R1, and/or VDR. Non-limiting examples of genetic variations within genes encoding LOC730217, OPRK1, OR6X1, DOCK10, CDH13, Cap S, IL4, ADAM33, IRS2, ABHD13, LINC00299, IL18, CYP2R1, and VDR include the SNVs listed in Table 35.

TABLE 35

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 180 | rs10142119 | 14 | 98486545 | G | A | LOC730217 | 0.52 | 2E−07 | 0.67 |
| 181 | rs1425902 | 8 | 54119214 | G | A | OPRK1 | 0.26 | 1E−06 | 0.76 |
| 182 | rs17744026 | 11 | 123648333 | T | G | OR6X1 | 0.91 | 3E−06 | 1.39 |
| 183 | rs1843834 | 2 | 225558042 | A | G | DOCK10 | 0.18 | 4E−06 | 0.76 |
| 184 | rs6563898 | 16 | 83358776 | G | A | CDH13 | 0.52 | 8E−06 | 0.60 |
| 185 | rs146456111 | 1 | 150705585 | C | A | Cap S | 0.43 | 1E−03 | 0.78 |
| 186 | rs2243250 | 5 | 132009154 | T | C | IL4 | 0.77 | 1E−03 | NR |

### Mental Traits

Disclosed herein, in some embodiments is a mental trait comprising a trait related to the mental health or mental acuity of the individual, mental illness, mental condition. Non-limiting examples of mental health or mental acuity includes a level of stress, short term memory retentions, long term memory retention, creative or artistic (e.g., “right-brained”), analytical and methodical (e.g., “left-brained”). Non-limiting examples of mental illness include schizophrenia, bipolar disorder, manic depressive disorder, autism spectrum disorder, and Down syndrome. Non-limiting examples of a mental condition include depression risk, social anxiety, likelihood of being an introvert, likelihood of being an extrovert. Non-limiting examples of a mental trait include morning person, empathy, worrier personality, mathematical ability, addictive personality, memory performance, OCD predisposition, exploratory behavior, reading ability, experiential learning difficulty, general creativity, general intelligence, impulsivity, inattentive symptoms, mathematical ability, mental reaction time, musical creativity, nail biting, reading and spelling difficulty, verbal and numerical reasoning and misophonia.

In some embodiments, the mental trait comprises memory performance. Memory performance may be affected by genetic variations within genes encoding APOC1, APOE, FASTKD2, MIR3130-1, MIR3130-2, SPOCK3, ANXA10, ISL1, PARP8, BAIAP2, HS3ST4, C16orf82, AJAP1, C1orf174, ODZ4, NARS2, PRR16, FTMT, PCDH20, TDRD3, LBXCOR1, MAP2K5, PTGER3, ZRANB2, AXUD1, TTC21A, GFRA2, DOK2, SLC39A14, PPP3CC, VPS26B, NCAPD3, ZNF236, MBP, RIN2, NAT5, SEMASA, MTRR, DGKB, ETV1, BHLHB5, CYP7B1, TMEPAI, ZBP1, TBC1D1, KLHL1, DACH1, LRRTM4, C2orf3, B3GAT1, LOC89944, ATP8B4, SLC27A2, CHD6, EMILIN3, RWDD3, TMEM56, SCN1A, KIBRA, and/or NCAN. Non-limiting examples of genetic variations within genes encoding APOC1, APOE, FASTKD2, MIR3130-1, MIR3130-2, SPOCK3, ANXA10, ISL1, PARP8, BAIAP2, HS3ST4, C16orf82, AJAP1, C1orf174, ODZ4, NARS2, PRR16, FTMT, PCDH20, TDRD3, LBXCOR1, MAP2K5, PTGER3, ZRANB2, AXUD1, TTC21A, GFRA2, DOK2, SLC39A14, PPP3CC, VPS26B, NCAPD3, ZNF236, MBP, RIN2, NAT5, SEMASA, MTRR, DGKB, ETV1, BHLHB5, CYP7B1, TMEPAI, ZBP1, TBC1D1, KLHL1, DACH1, LRRTM4, C2orf3, B3GAT1, LOC89944, ATP8B34, SLC27A2, CHD6, EMILIN3, RWDD3, TMEM56, SCN1A, KIBRA, and NCAN include the SNVs listed in Table 36.

TABLE 36

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 196 | rs4420638 | 19 | 45422946 | A | G | APOC1, APOE | 0.82 | 1E−16 | 8.27 |
| 197 | rs7594645 | 2 | 207646674 | G | A | FASTKD2, | 0.07 | 4E−09 | 0.07 |
| | | | | | | MIR3130-1, | | | |
| | | | | | | MIR3130-2 | | | |
| 198 | rs6813517 | 4 | 168522751 | T | C | SPOCK3, ANXA10 | 0.79 | 3E−08 | 0.37 |
| 199 | rs10058621 | 5 | 50555169 | T | C | ISL1, PARP8 | 0.94 | 3E−08 | 0.76 |
| 200 | rs8067235 | 17 | 79024637 | A | G | BAIAP2 | 0.33 | 6E−08 | 0.15 |
| 201 | rs11074779 | 16 | 26451443 | T | C | HS3ST4, C16orf82 | 0.81 | 1E−07 | 0.38 |
| 202 | rs932350 | 1 | 4853688 | T | C | AJAP1, C1orf174 | 0.32 | 2E−07 | 0.11 |

In some embodiments, the mental condition comprises obsessive compulsive disorder (OCD) predisposition. OCD predisposition may be affected by genetic variations within genes encoding PTPRD, LOC646114, LOC100049717, FAIM2, AQP2, TXNL1, WDR7, CDH10, MSNL1, GRIK2, HACE1, DACH1, MZT1, DLGAP1, EFNA5, and/or GRIN21B. Non-limiting examples of genetic variations within genes encoding PTPRD, LOC646114, LOC100049717, FAIM2, AQP2, TXNL1, WDR7, CDH10, MSNL1, GRIK2, HACE1, DACH1, MZT1, DLGAP1, EFNA5, and GRIN21B include the SNVs listed in Table 37.

TABLE 37

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 203 | rs4401971 | 9 | 11890045 | G | A | PTPRD, LOC646114, | 0.59 | 4E−07 | NR |
| | | | | | | LOC100049717 | | | |
| 204 | rs297941 | 12 | 50319086 | A | G | FAIM2, AQP2 | 0.53 | 5E−07 | 0.21 |
| 205 | rs12959570 | 18 | 54333584 | G | A | TXNL1, WDR7 | 0.23 | 9E−07 | 0.18 |
| 206 | rs6876547 | 5 | 25572301 | G | T | CDH10, MSNL1 | 0.19 | 2E−06 | NR |
| 207 | rs9499708 | 6 | 104445367 | T | C | GRIK2, HACE1 | 0.67 | 3E−06 | 0.18 |
| 208 | rs9652236 | 13 | 72688774 | T | G | DACH1, MZT1 | 0.18 | 5E−06 | 0.34 |
| 209 | rs11081062 | 18 | 3662879 | T | C | DLGAP1 | 0.36 | 4E−04 | 0.81 |

### Hair Trait

Disclosed herein, in some embodiments, are hair traits. In some embodiments, a hair trait comprises hair thickness, hair thinning, hair loss, baldness oiliness, dryness, dandruff, pseudofolliculitis barbae (razor bumps), monilethrix, pili trianguli, pili torti, and/or hair volume. In some embodiments, the term “baldness,” as used herein, refers to androgenetic alopecia (AGA). In some embodiments, the pili trianguli may be affected by genetic variations within genes encoding PADI3, TGM3, and/or TCHH. In some embodiments, the pseudofolliculitis barbae may be affected by genetic variations within genes encoding K6HF. In some embodiments, the monilethrix may be affected by genetic variations within genes encoding KRT81, KRT83, KRT86, and/or DSG4. In some embodiments, pili torti may be affected by genetic variants within genes encoding BCS1L. In some embodiments, baldness may be affected by genetic variations within genes encoding PAX1, TARDBP, HDAC4, HDAC9, AUTS2, MAPT-AS1, SPPL2C, SETBP1, GRID1, WNT10A, EBF1, SUCNR1, MBNL1, SSPN, ITPR2, AR, EDA2R, EDA2R, ICOS, CTLA4, IL2, IL21, ULBP3, ULBP6, STX17, IL2RA, PRDX5, IKZF4, and/or HLA-DQA2. Non-limiting genes that affect baldness include, but are not limited to, the SNVs listed in Table 38.

TABLE 38

| | Chr | Position | R RISK | NONRISK | | RISK ALLELE | | |
|---|---|---|---|---|---|---|---|---|
| SNV | (Build 37) | (Build 37) | ALLELE | ALLELE | GENE | FREQUENCY | P VALUE | BETA |
| rs2497938 | X | 66563018 | T | C | AR | 0.85 | 2E−91 | 79 |
| rs6047844 | 20 | 22037575 | T | C | PAX1, FOXA2 | 0.46 | 2E−39 | .47 |
| rs9275572 | 6 | 32678999 | G | A | HLA-DQA2 | 0.59 | 1E−35 | .79 |
| rs9479482 | 6 | 150358012 | A | G | ULBP3, ULBP6 | 0.57 | 4E−19 | .50 |
| rs1385699 | X | 65824986 | T | C | EDA2R | 0.70 | 4E−19 | .54 |
| rs2180439 | 20 | 21853100 | T | C | BQ013595, PAX1, | 0.57 | 3E−15 | .60 |
| | | | | | BE789145 | | | |
| rs7349332 | 2 | 219756383 | T | C | WNT10A | NR | 4E−15 | .29 |

### Behavioral Modifications

Aspects disclosed herein provide methods and systems for recommending to an individual a behavioral modification related to a specific phenotypic trait, based at least in part, on the genetic risk score (GRS) for that trait. In some instances, a plurality of recommendations of behavior modifications are provided to the individual. In some instances, a survey of the individual is provided by the individual comprising questions related to the specific phenotypic trait of interest. In some instances, the behavior modifications are based on the GRS for the trait, and the answers to the questions received from the individual. In some instances, the behavior modification comprises increasing, reducing, or avoiding an activity. Non-limiting examples of activities include, but are not limited to, comprising a physical exercise, ingestion of a substance (e.g., supplement or drug), exposure to a product (e.g., fumes, toxins, irritants, and the like), usage of a product (e.g., skin care product, hair care product, nail care product, and the like), a diet, a lifestyle, sleep, and consumption (e.g., consumption of alcohol, a drug, caffeine, an allergen, a food or category of foods). In some instances, the behavior modification comprises an activity to remedy or prevent the specific phenotypic trait (for e.g., engaging or not engaging in an activity that serves as a cause or a correlative to the occurrence of the specific phenotypic trait).

The present disclosure provides, by way of non-limiting examples, various recommendations of behavior modifications related to the specific phenotypic traits described herein. In some embodiments, an individual with a GRS indicating an increased likelihood for dry skin, as compared to a subject population, is recommended to engage in an activity to remedy and/or prevent dry skin (e.g., apply moisturizer on a daily basis). In some embodiments, an individual with a GRS indicating an increased likelihood for collagen breakdown, as compared to a subject population, is recommended to engage in an activity to remedy and/or prevent collagen breakdown (e.g., consumption of collagen supplement, use of a particular product or device, avoidance of a particular product or device). In some embodiments, an individual with a GRS indicating an increased likelihood of exercise aversion, as compared to a subject population, is recommended to engage in non-conventional physical activity (e.g., hobbies such as rock-climbing, hiking, backpacking, and the like). In some embodiments, an individual with a GRS indicating an increased likelihood for muscle damage risk, as compared to a subject population, is recommended to avoid activity to remedy or prevent muscle damage (e.g., body building, extreme endurance events, and the like). In some embodiments, an individual with a GRS indicating an increased likelihood for stress fractures, as compared to a subject population, is recommended to avoid activity to remedy of prevent stress fractures (e.g., repetitive and/or high-impact activities such as running). In some embodiments, an individual with a GRS indicating an increased likelihood to metabolize alcohol poorly, as compared to a subject population, is recommended to avoid consumption of alcohol, or to reduce alcohol consumption. In some embodiments, the subject population is ancestry-specific to the individual.

Reports

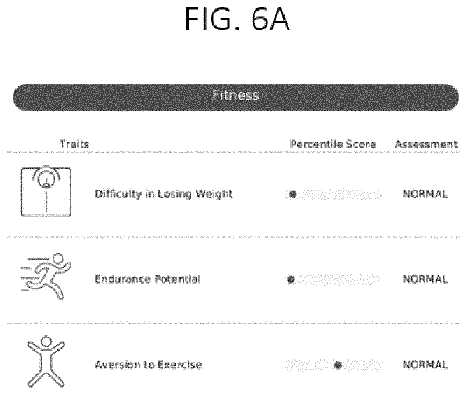

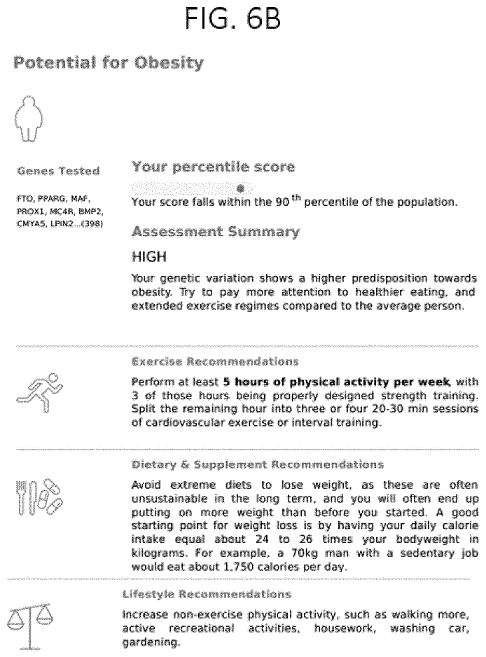

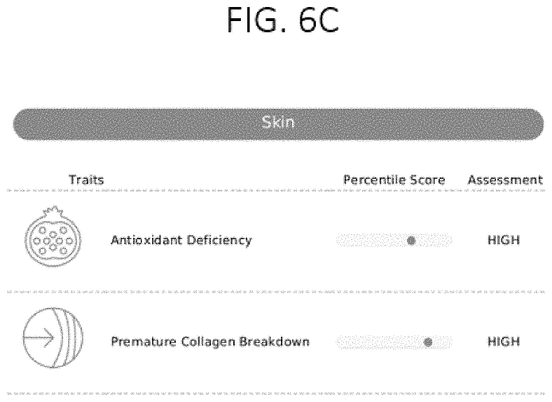

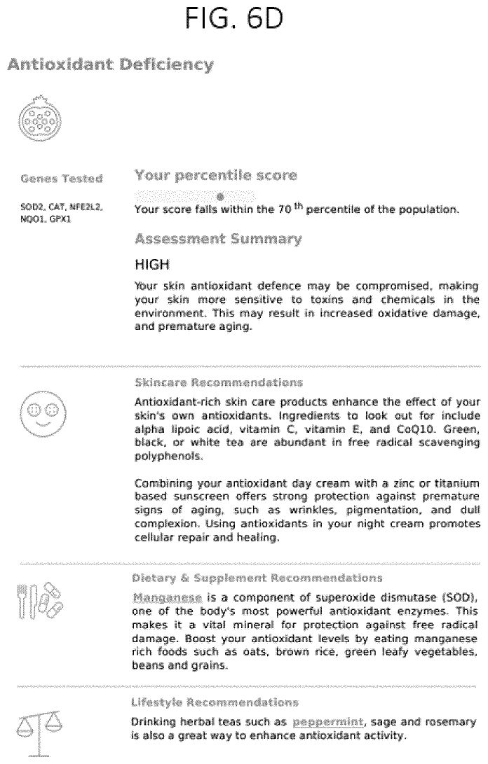

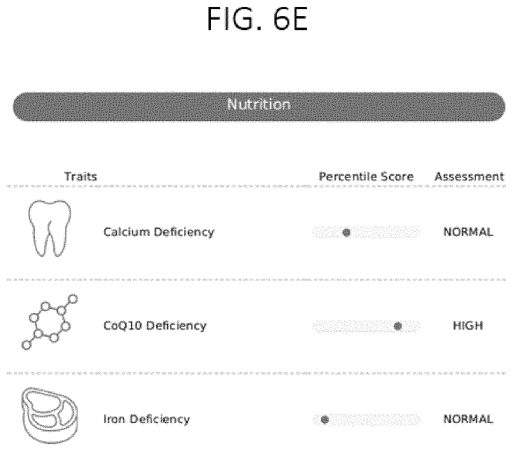

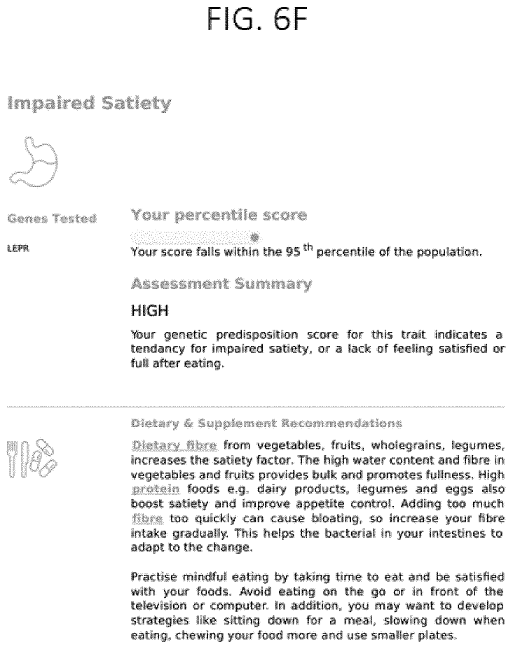

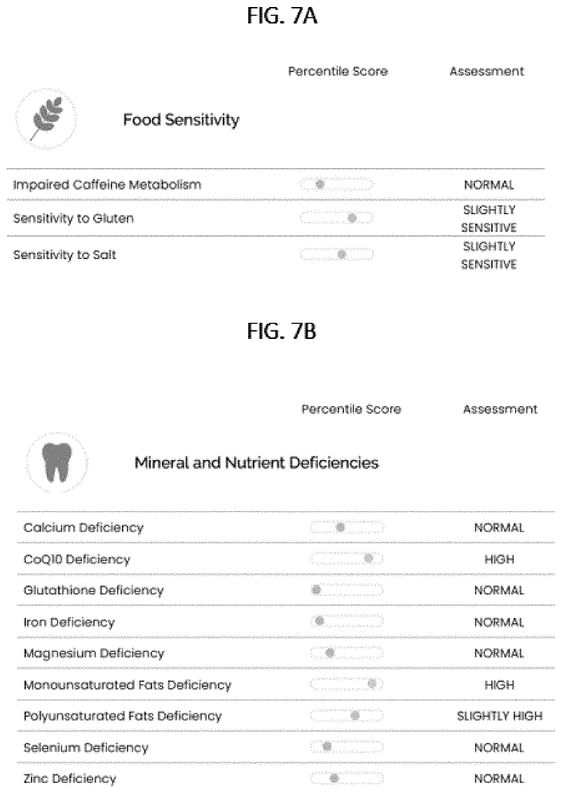

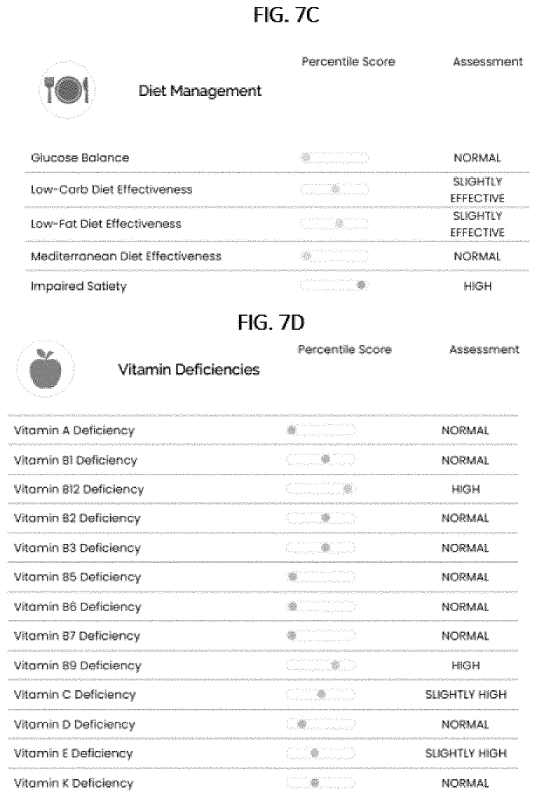

Disclosed herein, in some embodiments, are reports, such as wellness reports. Non-limiting examples of reports of the present embodiments are provided in FIG. 6A-6F and FIG. 7A-7D. The reports are generated using the methods and systems described herein, to provide the individual with results from the ancestry-specific genetic risk score (GRS) analysis of the genotype of the individual for one or more specific phenotypic traits described herein. In some cases, the reports comprise a recommendation to the individual, such as a behavior modification or product recommendation based on the GRS of the individual.

In some embodiments, the report comprises a result from the GRS analysis that is represented in a range (e.g., normal to high) of risk for developing or having the specific phenotypic trait of interest, which is relative to a reference population. In some cases, the reference population made up of individuals of the same ancestry as the individual. In some cases, the reference population is not ancestry-specific to the individual. In general, a “normal” result indicates that the individual is not predisposed to developing or having the phenotypic trait. In contrast, a “high” result indicates that the individual has a higher likelihood to develop or have the phenotypic trait, as compared to the reference population. A “low” risk indicates that the individual is predisposed not to have or develop the specific phenotypic trait. A “slightly high” or “slightly low” result indicates a score between a normal score and a high or a low score, respectively.

The reports described herein, in some cases, provide product recommendations based on the GRS of the individual for the specific phenotypic trait. In a non-limiting example, an individual predisposed to developing premature collagen breakdown (e.g., score in the 50^{th} percentile or more) would be recommended a product to restore, stop, or prevent collagen breakdown, such as collagen supplement. In various embodiments, the reports also comprise a hyperlink for the product that is recommended. The hyperlink will direct the individual to an online resource related to that product, such as an online commerce platform to purchase the product, or a research article or literature review article related to the specific phenotype.

Reports disclosed herein, in some embodiments, provide the individual with GRS results for multiple specific phenotypic traits, such as those described herein. For example, a single report in some cases includes results for one or more specific phenotypic traits related to one or more of skin, fitness, nutrition, and others, such as those provided in FIG. 6A-6F and FIG. 7A-7D and described herein.

The reports are formatted for delivery to the individual using any suitable method, including electronically or by mail. In some embodiments, the reports are electronic reports. Electronic reports, in some cases, are formatted to transmit via a computer network to a personal electronic device of the individual (e.g., tablet, laptop, smartphone, fitness tracking device). In some cases, the report is integrated into a mobile application on the personal electronic device. In some cases, the App is interactive, and permits the individual to click on hyperlinks embedded within the report that automatically redirect the user to an online resource. In some cases, the reports are encrypted or otherwise secured to protect the privacy of the individual. In some cases, the reports are printed and mailed to the individual.

Systems

Aspects disclosed herein provide systems configured to implement the methods described in this disclosure, including, but not limited to, determining a likelihood that an individual has, or will develop a specific phenotypic trait.

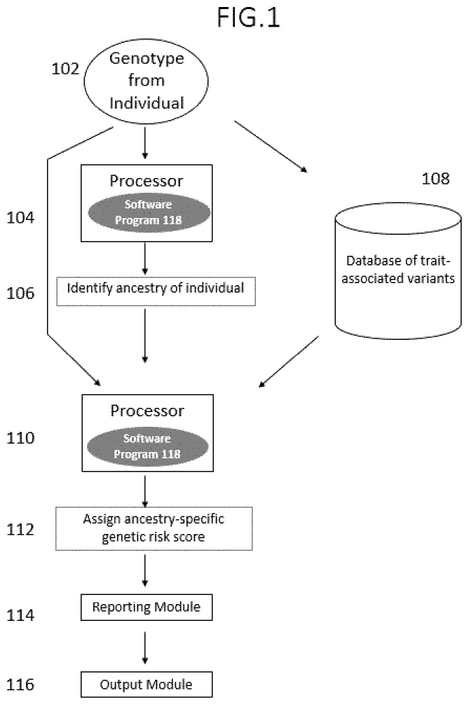

FIG. 1 describes exemplary wellness reporting systems comprising a computing device comprising at least one processor 104, 110, a memory, and a software program 118 including instructions executable by at least one processor to assess a likelihood that an individual has, or will develop, a specific phenotypic trait. In some instances, the system comprises a reporting module configured to generate a report the GRS to the individual. In some instances, the report comprises a recommendation of a behavioral modification related to the specific phenotypic trait. In some instances, the system comprises an output module configured to display the report to the individual. In some instances, the system comprises a central processing unit (CPU), memory (e.g., random access memory, flash memory), electronic storage unit, software program, communication interface to communicate with one or more other systems, and any combination thereof. In some instances, the system is coupled to a computer network, for example, the Internet, intranet, and/or extranet that is in communication with the Internet, a telecommunication, or data network. In some instances, the system is connected to a distributed ledger. In some instances, the distributed ledger comprises blockchain. In some embodiments, the system comprises a storage unit to store data and information regarding any aspect of the methods described in this disclosure. Various aspects of the system are a product or article or manufacture.

The exemplary wellness reporting systems of FIG. 1, comprise one feature of a software program that includes a sequence of instructions, executable by the at least one processor, written to perform a specified task. In some embodiments, computer readable instructions are implemented as program modules, such as functions, features, Application Programming Interfaces (APIs), data structures, and the like, that perform particular tasks or implement particular data types. In light of the disclosure provided herein, those of skill in the art will recognize that a software program may be written in various versions of various languages. In some embodiments, the software program 118 includes instructions executable by the at least one processor described herein. In some embodiments, the instructions comprise the steps of: (i) providing the genotype of the individual, the genotype comprising one or more individual-specific genetic variants; (ii) assigning an ancestry to the individual based, at least in part, on the genotype of the individual 106; (iii) using a trait-associated variants database 108 comprising ancestry-specific genetic variants derived from subjects with the same ancestry as the individual (subject group) to select one or more ancestry-specific genetic variants based, at least in part, on the ancestry of the individual, wherein each of the one or more ancestry-specific genetic variants correspond to: (1) an individual-specific genetic variant of the one or more individual-specific genetic variants, or (2) a predetermined genetic variant in a linkage disequilibrium (LD) with an individual-specific genetic variant of the one or more individual-specific genetic variants in a subject population with the same ancestry as the individual, and wherein each of the one or more ancestry-specific genetic variants and each of the individual specific genetic variants comprises one or more units of risk; and (iv) calculating a genetic risk score 112 for the individual based on the selected one or more ancestry-specific genetic variants, wherein the genetic risk score is indicative of the likelihood that the individual has, or will develop the specific trait. In some embodiments, the software program 118 further comprises instructions executable by the at least one processor described herein comprising predetermining a genetic variant in LD with the individual-specific genetic variant. In some instances, the software program includes instructions executable by the at least one processor to determine the predetermined genetic variant, the instructions comprising the steps of: (i) providing unphased genotype data from an individual; (ii) phasing the unphased genotype data to generate individual-specific phased haplotypes based on the ancestry of the individual; (iii) imputing individual-specific genotypes not present in the phased individual-specific phased haplotypes using phased haplotype data from a reference group that has the same ancestry as the individual; and (iv) selecting a genetic variant from the imputed individual-specific genotypes that is in linkage disequilibrium (LD) an individual-specific genetic variant associated with a likelihood that the individual has, or will develop, a specific trait. In some embodiments, the LD is defined by a D′ value at least about 0.20, 0.25, 0.30, 0.35, 0.40, 0.45, 0.50, 0.55, 0.60, 0.65, 0.70, 0.75, 0.80, 0.85, 0.90, 0.95 or 1.0. In some embodiments, the LD is defined by a r² value at least about 0.70, 0.75, 0.80, 0.85, 0.90, 0.95, or 1.0.

The functionality of the computer readable instructions are combined or distributed as desired in various environments. In some instances, a software program comprises one sequence of instructions or a plurality of sequences of instructions. A software program may be provided from one location. A software program may be provided from a plurality of locations. In some embodiment, a software program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

FIG. 1 describes an exemplary wellness reporting system comprising a reporting module 114. The reporting module 114 described herein comprises at least one processor configured to perform the task of generating a report comprising the calculated GRS of the individual indicative of a likelihood that the individual has, or will develop, a specific phenotypic trait of interest. In some instances, the at least one processor is the same processor 118 described above, and additionally configured to perform the steps of generating the report. In some instances, the at least one processor comprises a separate processor, such as in a dual-CPU. In some instances, the reporting module 114 is configured to perform the task of retrieving one or more answers to one or more questions relating to the specific trait in a survey provided to the system by the individual. In some instances, the report further comprises a recommendation of a behavioral modification related to the trait based, at least in part, on the GRS. In some instances, the report generated by the reporting module 114 comprises a recommendation of a behavior modification related to the specific phenotypic trait of interest based on the GRS for that trait and retrieved one or more answer to the one or more questions relating to the trait.

In some embodiments, the exemplary wellness reporting systems of FIG. 1 comprise an output module 116. The output module 116 described herein comprises a hardware, or software program capable of being performed on a processor, configured to display the report to the individual. In some embodiments, the output module 116 comprises user interface, including a screen, or other output display (e.g., projector). In some embodiments, the output module 116 comprises emailing service capable of emailing an electronic version of the report to the individual to which it belongs. In some embodiments, the output module 116 comprises a user interface on a personal computing device, such as a computer, smartphone, or tablet. In some embodiments, the personal computing device is remotely connected, via a computer network, to the system described herein. In some instances, the personal computing device belonging to the individual. In some embodiments, the personal electronic device is configured to run an application configured to communicate with the reporting module via a computer network to access the report.

### Web Application

In some embodiments, the software programs described herein include a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, software programs described herein include a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, the software programs described herein include a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Perl, R, Python™, Visual Basic, and VB .NET, or combinations thereof. Compilation may be often performed, at least in part, to create an executable program. In some instances, a computer program includes one or more executable complied applications.

### Web Browser Plug-in

Disclosed herein, in some embodiments, are software programs that, in some aspects, include a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands. Those skilled in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, MicrosoftR Internet ExplorerR Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, such as the trait-associated database described herein, or use of the same. Those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The methods, systems, and media described herein, are configured to be performed in one or more facilities at one or more locations. Facility locations are not limited by country and include any country or territory. In some instances, one or more steps of a method herein are performed in a different country than another step of the method. In some instances, one or more steps for obtaining a sample are performed in a different country than one or more steps for analyzing a genotype of a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a sample of a subject and any article or product disclosed herein as an article or product. Data includes, but is not limited to, information regarding genotype and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

### Non-Transitory Computer Readable Storage Medium

Aspects disclosed herein provide one or more non-transitory computer readable storage media encoded with a software program including instructions executable by the operating system. In some embodiments, software encoded includes one or more software programs described herein. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Kits and Articles of Manufacture

Disclosed herein, in some embodiments, are compositions useful for the detection of a genotype or biomarker in a sample obtained from a subject according to the methods described herein. Aspects disclosed herein provide compositions comprises a polynucleotide sequence comprising at least 10 but less than 50 contiguous nucleotides of one or more of SEQ ID NOS: 1-218, or reverse complements thereof, wherein the contiguous polynucleotide sequence comprises a detectable molecule. In some embodiments, the polynucleotide sequence comprises the nucleobase at position 26 or 31 in one or more of SEQ ID NOS: 1-218. In various embodiments, the detectable molecule comprises a fluorophore. In other embodiments, the polynucleotide sequences further comprise a quencher.

Also disclosed herein, in some embodiments, are kits useful for to detect the genotypes described herein. In some embodiments, the kits disclosed herein may be used to predict whether an individual has, or will develop, a specific phenotype trait. In some instances, the kits are useful to diagnose or prognose a disease or condition in an individual. In some instances, the kits are useful for selecting a patient for treatment. In some cases, the kit is accompanied by a product recommendation, such as a supplement, or over-the-counter medication. In some cases, the kit is accompanied by a recommendation to consult a physician or medical healthcare professional.

In some embodiments, the kit comprises the compositions described herein, which can be used to perform the methods of detecting the genotypes described herein. Kits comprise an assemblage of materials or components, including at least one of the compositions. In other embodiments, the kits contains all of the components necessary and/or sufficient to perform an assay for detecting the genotypes, including all controls, directions for performing assays, and any necessary software for analysis and presentation of results. The kits disclosed herein are, in some cases, suitable for assays such as PCR, and qPCR. In some cases, the kit comprises a genotyping chip that can be used at the point of need. The exact nature of the components configured in the kit depends on its intended purpose.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Certain Terminologies

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by about 10%, 5%, or 1%.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “increased,” or “increase” are used herein to generally mean an increase by a statically significant amount; in some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

“Ancestry” as disclosed herein, refers to the genetic lineage of an individual.

The term, “genotype” as disclosed herein, refers to the chemical composition of polynucleotide sequences within the genome of an individual.

“Treatment” and “treating” as used herein refer to both therapeutic treatment and prophylactic or preventative measures, wherein the object is to prevent or slow down (lessen) the targeted condition, prevent the condition, pursue or obtain good overall result, or lower the chances of the individual developing the condition even if the treatment is ultimately unsuccessful. In some aspects provided herein, subjects in need of treatment include those already with a disease or condition, as well as those susceptible to develop the disease or condition or those in whom the disease or condition is to be prevented. In some instances, the treatment comprises a supplement. Non-limiting examples of a supplement includes a vitamin, a mineral, an antioxidant, a probiotic, and an anti-inflammatory. In some instances, the treatment comprises a drug therapy. In some instances, the drug therapy comprises an antibiotic, or an antibody or small molecule compound targeting a gene, or gene expression product thereof, disclosed herein.

“Genotype” or “genotypes” as disclosed herein, refers to the chemical composition of polynucleotide sequences within the genome of an individual. In some embodiments, the genotype comprises SNVs, single nucleotide polymorphisms (SNPs), indels, and/or CNVs. The term, “single nucleotide variant” or “single nucleotide variation” or SNV, as disclosed herein, refers to a variation in a single nucleotide within a polynucleotide sequence. The variation of an SNV may have multiple different forms. A single form of an SNV is referred to as an “allele.” By way of example, a reference polynucleotide sequence reading 5′ to 3′ is TTACG. A SNV at allele position 3 (of 5′-TTACG-3′) comprise a substitution of the reference allele, “A” to a non-reference allele, “C.” If the “C” allele of the SNV is associated with an increased probability of developing a phenotypic trait, the allele is considered a “risk” allele. However, the same SNV may also comprise a substitution of the “A” allele to a “T” allele. If the T allele of the SNV is associated with a decreased probability of developing a phenotypic trait, the allele is considered a “protective” allele. The SNV may comprise a single nucleotide polymorphism (SNP), in some cases, is an SNV observed in at least 1% of a given population. In some embodiments, the SNV is represented by an “rs” number, which refers to the accession of reference cluster of one more submitted SNVs in the dbSNP bioinformatics database, and which is characterized by a sequence that comprises the total number of nucleobases from 5′ to 3′, including the variation that was submitted. In some embodiments, a SNV may be further defined by the position of the SNV (nucleobase) within a provided sequence, the position of which is always located at the 5′ length of the sequence plus 1. In some embodiments, a SNV is defined as the genomic position in a reference genome and the allele change (e.g. chromosome 7 at position 234,123,567 from G allele to A allele in the reference human genome build 37). In some embodiments, the SNV is defined as the genomic position identified with a non-nucleotide letter or code (e.g., IUPAC nucleotide code) in a sequence disclosed herein.

“Indel,” as disclosed herein, refers to an insertion, or a deletion, of a nucleobase within a polynucleotide sequence. In some embodiments, the indel is represented by an “rs” number, which refers to the accession of reference cluster of one more submitted indels in the dbSNP bioinformatics database, and which is characterized by a sequence that comprises the total number of nucleobases from 5′ to 3′, including the variation that was submitted. In some embodiments, a indel may be further defined by the position of the insertion/deletion within a provided sequence, the position of which is always located at the 5′ length of the sequence plus 1. In some embodiments, an indel is defined as the genomic position in a reference genome and the allele change. In some embodiments, the indel is defined as the genomic position identified with the non-nucleotide letter or code (e.g., IUPAC nucleotide code) in a sequence disclosed herein.

“Copy number variant” or “copy number variation” or “CNV” disclosed herein, refers a phenomenon in which sections of a polynucleotide sequence are repeated or deleted, the number of repeats in the genome varying between individuals in a given population. In some embodiments, the section of the polynucleotide sequence is “short,” comprising about two nucleotides (bi-nucleotide CNV) or three nucleotides (tri-nucleotide CNV). In some embodiments, the section of the polynucleotide sequence is “long,” comprising a number of nucleotides between four nucleotides and an entire length of a gene.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

### EXAMPLES

### Example 1. Calculating an Ancestry-Specific Genetic Risk Score for an Individual Representing a Likelihood that the Individual Will have Better Aerobic Performance

First, a genotype of an individual is provided. The genotype of the individual may be in the format of an Illumina Genotyping Array. The genotype includes genetic risk variants specific to the individual (individual-specific genetic risk variants). The genetic risk variants may include single nucleotide variants (SNVs), single nucleotide polymorphisms (SNPs), indels, and/or copy-number variants (CNVs). The Illumina Genotyping Array comprises nucleic acid probes specific to various SNVs, indels, SNPs, and/or CNVs. Using principal component analysis (PCA), the genotype is analyzed to determine the ancestry of the individual, and the individual is determined to be of African descent.

Next, reference genetic variants are selected from genome wide associate studies (GWAS) of subjects with the same ancestry as the individual (e.g., African)(ancestry-specific subject group), as determined by PCA. The ancestry-specific variants are located at reported susceptibility genetic loci for aerobic performance comprising TSHR, ACSL1, PRDM1, DBX1, GRIN3A, ESRRB, ZIC4, and/or CDH13, and are selected based on a strong association (P=1.0×10⁻⁴ or lower) between the ancestry-specific genetic variants and the aerobic performance trait. The variants are provided in Table 39.

TABLE 39

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 54 | rs7144481 | 14 | 81610942 | C | T | TSHR | NR | 9E−08 | NR |
| 55 | rs6552828 | 4 | 185725416 | G | A | ACSL1 | NR | 1E−06 | NR |
| 56 | rs10499043 | 6 | 106247137 | A | G | PRDM1 | 0.13 | 4E−06 | NR |
| 57 | rs10500872 | 1 | 20245723 | A | G | DBX1 | NR | 6E−06 | NR |
| 58 | rs1535628 | 9 | 105016749 | G | A | GRIN3A | 0.09 | 7E−06 | NR |
| 59 | rs12893597 | 4 | 76812695 | T | C | ESRRB | NR | 7E−06 | NR |
| 60 | rs11715829 | 3 | 146957166 | A | G | ZIC4 | 0.08 | 9E−06 | NR |

If an individual-specific genetic risk variant is unknown, meaning the identification number of the genotyping array corresponding to the individual-specific genetic variant is unpublished in the GWAS above, a proxy genetic variant is selected to serve as the basis for the genetic risk calculations. A proxy genetic variant is selected, also known as “imputation,” if it is in linkage disequilibrium (LD) (r² value of at least 0.70 or D′ value of at least about 0.20) with the unknown individual-specific genetic risk variant.

Next, an individual-specific raw score is calculated. Numerical values are assigned to units of risk (e.g., risk alleles) within the individual-specific genetic variants, and all numerical values for each individual-specific genetic variant are added together, and divided by the total number of the individuals-specific genetic variants and/or proxy genetic variants to generate an individual-specific raw score.

Next, the same calculations are performed to generate a raw score for each individual within the ancestry-specific subject group, thereby generating an observed range of raw scores (observed range). Next, the individual-specific raw score is compared to the ancestry-specific observed range to calculate a percentage of risk relative to the ancestry-specific subject population. Next, a genetic risk score (GRS) is assigned to the individual.

For example, to calculate the GRS for an individual for aerobic performance comprised of seven genetic variants, in this example SNPs (rs7144481 with risk allele C, rs6552828 with risk allele G, rs1049904 with risk allele A, rs10500872 with risk allele A, rs1535628 with risk allele G, rs1289359 with risk allele T, and rs1171582 with risk allele A) requires that each genotype be determined by actual genotyping or imputation and that the average of the sum of all risk alleles be calculated. Hence, an individual with genotypes rs7144481 (CC), rs6552828 (AA), rs1049904 (GG), rs10500872 (AG), rs1535628 (AA), rs1289359, (CT), rs1171582 (AA) has risk alleles of 2, 0, 0, 1, 0, 1, and 2, respectively, resulting in a sum of 6 with an average genetic risk score of 0.86 (=6/7; risk alleles divided by the total number of variants comprising the model). Table 40 provides exemplary calculations in accordance with the example provided.

TABLE 40

| | Risk | Non-risk | Individual's | Number of |
|---|---|---|---|---|
| Variant | allele | allele | genotype | risk alleles |
| rs7144481 | C | T | CC | 2 |
| rs6552828 | G | A | AA | 0 |
| rs1049904 | A | G | GG | 0 |
| rs10500872 | A | G | AG | 1 |
| rs1535628 | G | A | AA | 0 |
| rs1289359 | T | C | CT | 1 |
| rs1171582 | A | G | AA | 2 |
| | | | Total number of | 6 |
| | | | risk alleles | |
| | | | Average number of risk | 0.86 |
| | | | alleles (6 risk alleles | |
| | | | divided by 7 variants | |
| | | | comprising the model) | |

The GRS score is similarly calculated for the ancestry-specific population. When the individual's GRS score is compared to the distribution of GRS scores from the same ancestry-specific population, the individual's GRS score is in the 50^{th} percentile. The individual is predicted to have average aerobic performance.

### Example 2. Calculating an Ancestry-Specific Genetic Risk Score for an Individual Representing a Likelihood that the Individual Will Experience Collagen Breakdown

First, a genotype of an individual is provided. The genotype of the individual may be in the format of an Illumina Genotyping Array. The genotype includes genetic risk variants specific to the individual (individual-specific genetic risk variants). The genetic risk variants may include single nucleotide variants (SNVs), single nucleotide polymorphisms (SNPs), indels, and/or copy-number variants (CNVs). The Illumina Genotyping Array comprises nucleic acid probes specific to various SNVs, SNPs, and/or CNVs. Using principal component analysis (PCA), the genotype is analyzed to determine the ancestry of the individual, and the individual is determined to be Chinese.

Next, reference genetic variants are selected from GWAS. The variants are at reported susceptibility genetic loci MMP1, MMP3 and MMP9 for collagen breakdown and are selected based on strong association (P=1.0×10⁻⁴ or lower) between the genetic variations and the physical fitness trait. The variants are provided in Table 41.

TABLE 41

| SEQ ID | | Chr | Position | RISK | NONRISK | | RISK ALLELE | | |
|---|---|---|---|---|---|---|---|---|---|
| NO | SNV | (Build 37) | (Build 37) | ALLELE | ALLELE | GENE | FREQUENCY | P-VALUE | BETA |
| 1 | rs495366 | 11 | 102695108 | G | A | MMP | 0.64 | 6E−34 | 0.44 |
| 2 | rs11226373 | 11 | 104334239 | G | A | MMP-3, MMP-1 | 0.15 | 1E−18 | 0.44 |

If an individual-specific genetic risk variant is unknown, meaning the array identification number corresponding to the individual-specific genetic variant is unpublished in the GWAS above, a proxy genetic variant is selected to serve as the basis for the genetic risk calculations. A proxy genetic variant is selected if it is in linkage disequilibrium (LD) (r² value of at least 0.70 or D′ value of at least about 0.20 based on subjects with the same ancestry as the individual) with the unknown individual-specific genetic risk variant.

Next, an individual-specific raw score is calculated. Numerical values are assigned to units of risk (e.g., risk alleles) within the individual-specific genetic variants, and all numerical values for each individual-specific genetic variant are added together, and divided by the total number of individual-specific genetic variants or proxy genetic variants, to generate an individual-specific raw score.

Next, the same calculations are performed to generate a raw score for each individual within the ancestry-specific subject group, thereby generating an observed range of raw scores (observed range). Next, the individual-specific raw score is compared to the ancestry-specific observed range to calculate a percentage of risk relative to the ancestry-specific subject population. Next, a genetic risk score (GRS) is assigned to the individual.

For example, to calculate the GRS for an individual for a collagen breakdown trait comprised of two genetic variants, in this example SNPs (rs495366 with risk allele G, and rs11226373 with risk allele G) requires that each genotype be determined by actual genotyping or imputation and that the average of the sum of all risk alleles be calculated. Hence, an individual with genotypes rs495366 (GG), rs11226373 (GA) has risk alleles of 2, and 1, respectively, resulting in a sum of 3 with an average genetic risk score of 1.5 (=3/2; risk alleles divided by the total number of variants comprising the model). Table 42 provides exemplary calculations in accordance with the present example.

TABLE 42

| | Risk | Non-risk | Individual's | Number of |
|---|---|---|---|---|
| Variant | allele | allele | genotype | risk alleles |
| rs495366 | G | A | GG | 2 |
| rs11226373 | G | A | GA | 1 |
| | | | Total number of | 3 |
| | | | risk alleles | |
| | | | Average number of risk | 1.5 |
| | | | alleles (3 risk alleles | |
| | | | divided by 2 variants | |
| | | | comprising the model) | |

The GRS score is similarly calculated for the ancestry-specific population. When the individual's GRS score is compared to the distribution of GRS scores from the same ancestry-specific population, the individual's GRS score is in the 90^{th} percentile. The individual is predicted to have high risk of collagen breakdown and is advised to hydrate their skin and apply collagen cream.

### Example 3. Calculating an Ancestry-Specific Genetic Risk Score for an Individual Representing a Likelihood that the Individual Will Experience Vitamin a Deficiency

First, a genotype of an individual is provided. The genotype of the individual may be in the format of an Illumina Genotyping Array. The genotype includes genetic risk variants specific to the individual (individual-specific genetic risk variants). The genetic risk variants may include single nucleotide variants (SNVs), single nucleotide polymorphisms (SNPs), indels, and/or copy-number variants (CNVs). The Illumina Genotype Chip comprises nucleic acid probes specific to various SNVs, SNPs, indels, and/or CNVs. Using principal component analysis (PCA), the genotype is analyzed to determine the ancestry of the individual, and the individual is determined to be Chinese.

Next, reference genetic variants are selected from GWAS that was published in a high-impact journal. The variants are at reported susceptibility genetic loci BCMO1, FFAR4 and TTR for Vitamin A deficiency and are selected based on strong association(P=1.0×10⁻⁴ or lower) between the genetic variations and the nutrition trait. The ancestry-specific variants are provided in Table 43.

TABLE 43

| SEQ ID | | Chr | Position | Risk | Non-Risk | | Risk Allele | | |
|---|---|---|---|---|---|---|---|---|---|
| NO | SNV | (Build 37) | (Build 37) | Allele | Allele | Gene | Frequency | P-Value | Beta |
| 129 | rs6564851 | 16 | 81264597 | T | G | BCMO1 | 0.61 | 2E−24 | 0.15 |
| 210 | rs10882272 | 10 | 95348182 | C | T | FFAR4 | 0.35 | 7E−15 | 0.03 |
| 211 | rs1667255 | 18 | 29187279 | A | C | TTR | 0.31 | 6E−14 | 0.03 |

If an individual-specific genetic risk variant is unknown, meaning the array identification number corresponding to the individual-specific genetic variant is unpublished in the GWAS above, a proxy genetic variant is selected to serve as the basis for the genetic risk calculations. A proxy genetic variant is selected if it is in linkage disequilibrium (LD) (r² value of at least 0.70 or D′ value of at least about 0.20 based on subjects with the same ancestry as the individual) with the unknown individual-specific genetic risk variant.

Next, an individual-specific raw score is calculated. Numerical values are assigned to units of risk (e.g., risk alleles) within the individual-specific genetic variants, and all numerical values for each individual-specific genetic variant are added together, and divided by the total number of individual-specific genetic variants or proxy genetic variants, to generate an individual-specific raw score.

Next, the same calculations are performed to generate a raw score for each individual within the ancestry-specific subject group, thereby generating an observed range of raw scores (observed range). Next, the individual-specific raw score is compared to the ancestry-specific observed range to calculate a percentage of risk relative to the ancestry-specific subject population. Next, a genetic risk score (GRS) is assigned to the individual.

For example, to calculate the GRS for an individual for a vitamin A deficiency trait comprised of three genetic variants, in this example SNPs (rs6564851 with risk allele T, rs1082272 with risk allele C, and rs1667255 with risk allele A) requires that each genotype be determined by actual genotyping or imputation and that the average of the sum of all risk alleles be calculated. Hence, an individual with genotypes rs6564851 (TG), rs1082272 (TT), and rs1667255 (AC) has risk alleles of 1, 0, and 1, respectively, resulting in a sum of 2 with an average genetic risk score of 1.67 (=⅔; risk alleles divided by the total number of variants comprising the model). Table 44 provides exemplary calculations in accordance with the present example.

TABLE 44

| | Risk | Non-risk | Individual's | Number of |
|---|---|---|---|---|
| Variant | allele | allele | genotype | risk alleles |
| rs6564851 | T | G | TG | 1 |
| rs1082272 | C | T | TT | 0 |
| rs1667255 | A | C | AC | 1 |
| | | | Total number of | 2 |
| | | | risk alleles | |
| | | | Average number of risk | 0.67 |
| | | | alleles (2 risk alleles | |
| | | | divided by the 3 variants | |
| | | | comprising the model) | |

The GRS score is similarly calculated in the ancestry-specific population. When the individual's GRS score is compared to the distribution of GRS scores from the same ancestry-specific population, the individual's GRS score 1 standard deviation above the mean. The individual is predicted to be at risk for vitamin A deficiency and is advised to take vitamin A supplements.

### Example 4. Ancestry-Specific Genetic Risk Score for Alcohol Flush Reaction

Alcohol flush reaction is a condition in which a person develops flushes or blotches on the face, neck, shoulders, and in some cases, the entire body after consuming alcohol beverages. Approximately one-third of people of East Asian descent experience facial flushing from drinking. The single nucleotide polymorphism (SNP) rs671 allele A in the gene encoding alcohol dehydrogenase 1B (ADH1B) is associated with the flush reaction. This SNP is present in the trait-associated variants database disclosed herein. By way of example and not limitation, Tables 45-46 show the likelihood that an individual has or will develop alcohol flush reaction, by calculating the genetic risk score (GRS) using a predominant European reference population, as compared to calculating the GRS using a reference population made up of individuals of the same ancestry as the subject. Individuals with scores greater than or equal to 1 are predicted to have a moderate alcohol flush reaction because rs671 acts in a dominant negative fashion.

Cohort.

A dataset that includes genotypes from 1,669 individuals was analyzed. Within the dataset of 1,669 individuals, 193 were of European ancestry (EUR) and 1,476 were of East Asian ancestry (EAS). The ancestry-specific variant (rs671) is located at reported susceptibility genetic locus for alcohol dehydrogenase, ADH1B and was selected based on a strong association to the alcohol flush reaction trait in the reference populations.

Genotyping.

A sample of saliva was obtained from each subject. The sample was genotyped on the IlluminaCore-24 BeadChip platform (Illumina, Inc., San Diego, Calif. 92121). Quality control measures according to the manufacturing instructions for the IlluminaCore-24 platform were used. SNPs were excluded from analysis if: genotyping rate <93%; missingness in SNPs>10% and minor allele frequency <0.01.

Assigning Ancestry to the Subject.

Principal component analysis (PCA) of the genotypes was used to analyze population groupings. For each subject, a distance-based method (K-means) was used to calculate the nearest population to the sample and assign ancestry.

Imputation of rs671.

To score the alcohol reaction trait, rs671 genotypes were imputed. Imputation was done using reference populations from 1000 Genomes that were either ancestry-specific or not ancestry-specific for comparison.

Ancestry-Specific Reference Population.

A subject's genotypes were imputed based on its assigned ancestry. An ancestry-specific reference population was selected from the 1000 Genomes project described in “A global reference for human genetic variations,” Nature 526, 68-74 (Oct. 1, 2015). If a subject was assigned East Asian ancestry in the previous step, then its genotypes were imputed using the East Asian population from 1000 Genomes as the reference population. If a subject was assigned European ancestry in the previous step, then the subject's genotypes were imputed using the European population from 1000 Genomes as the reference population.

Non-Ancestry-Specific Reference Population.

To provide a comparison of the accuracy of the GRS using a reference population that is not ancestry specific, reference genetic variants were also selected the subject's genotypes were imputed using European population from 1000 Genomes as a reference population.

Calculation of GRS.

An analysis of subjects with alcohol flush scores based on rs671 imputed genotypes was performed using both the ancestry-specific reference population and the non-ancestry-specific reference population for comparison. A subject's alcohol flush reaction score was calculated based on the number of non-reference alleles.

Results.

Table 45 illustrates a pipeline using single reference population where ancestry is not considered, whereas Table 46 illustrates an ancestry-specific pipeline where the pipeline is specific to the ancestry of the individual. It is known that 36-45% of East Asians have facial flushing in response to drinking alcohol. This is accurately reflected in Table 46 where the ancestry-specific pipeline predicted that 42% of the individuals with East Asian ancestry have or will develop alcohol flush reaction. In contrast, Table 45 illustrates when Asian ancestry was not considered, none of the East Asian individuals were predicted to have the alcohol flush reaction which does not match what is known. “EUR” refers to Europeans, and “EAS” refers to East Asian.

TABLE 45
Pipeline using single reference population
(EUR). Ancestry not considered.

| | Prediction for | Individuals with | Individuals with East |
|---|---|---|---|
| | Alcohol Flush | European ancestry | Asian ancestry |
| Score | Reaction | (n = 193) | (n = 1476) |
| 0 | Average | 100% (193) | 100% (1476/1476) |
| 1 | Moderate | 0% (0) | 0% (0) |
| 2 | Moderate | 0% (0) | 0% (0) |

EUR: Europeans

TABLE 46
Ancestry-specific pipeline (pipeline specific to
ancestry of individual, where EUR is used for EUR
individuals and EAS is used for EAS individuals).

| | | Individuals with | Individuals with East |
|---|---|---|---|
| | | European ancestry | Asian ancestry % |
| Score | Prediction | (n = 193) | (n = 1476) |
| 0 | Average | 100% (193) | 58% (854) |
| 1 | Moderate | 0% (0) | 37% (549) |
| 2 | Moderate | 0% (0) | 5% (73) |

EUR: Europeans;
EAS: East Asian

### Example 5. Ancestry-Specific Genetic Risk Score for Lactose Tolerance

Lactose tolerant individuals are adults that can consume animal milk and animal milk products without risk of lactose intolerance symptoms such as bloating, pain, cramps, diarrhea, gas, or nausea. The genetic trait of lactose tolerance is associated with functional single nucleotide variants (SNVs) in a regulatory region about 14 kb upstream of the lactase gene (LCT), including −13910*T (rs4988235), −13915*G (rs41380347), and −14010*C (rs145946881). These three SNVs are present in the trait-associated variants database disclosed herein. By way of example and not limitation, Tables 47-48 show whether an individual is lactose tolerant or lactose intolerant, by calculating the genetic risk score (GRS) using a predominant European reference population, as compared to calculating the GRS using a reference population made up of individuals of the same ancestry as the subject. Individuals with scores equal to zero are predicted to be lactose intolerant and individuals with scores greater than zero are predicted to be lactose tolerant because one allele is sufficient to metabolize lactose.

Cohort. A dataset that includes 1,669 individuals was analyzed. Within the dataset of 1,669 individuals, 193 were of European ancestry (EUR) and 1,476 were of East Asian ancestry (EAS). The ancestry-specific variants 13910*T (rs4988235), −13915*G (rs41380347), and −14010*C (rs145946881) located at reported susceptibility genetic loci upstream of the LCT gene was selected based on a strong association to the alcohol flush reaction trait in the reference populations.

Genotyping.

A sample of saliva was obtained from the subject. The sample was genotyped on the IlluminaCore BeadChip platform (Illumina, Inc., San Diego, Calif. 92121). Quality control measures according to the manufacturing instructions for the IlluminaCore-24 platform were used. SNVs were excluded from analysis if: genotyping rate <93%; missingness in SNPs >10% and minor allele frequency <0.01.

Assigning Ancestry to the Subject.

Principal component analysis (PCA) of the genotypes was used to analyze population groupings. For each subject, a distance-based method (K-means) was used to calculate the nearest population sample and assign ancestry.

Imputation of rs4988235, rs41380347, and rs145946881.

To score lactose tolerance, rs4988235, rs41380347, and rs145946881 genotypes were imputed. Imputation was done using reference populations from 1000 Genomes that were either ancestry-specific or not ancestry specific for comparison.

Ancestry-Specific Reference Population.

A subject's genotypes were imputed based on its assigned ancestry. If a subject was assigned East Asian ancestry in the previous step, then its genotypes were imputed using the East Asian population from 1000 Genomes as the reference population. If a subject was assigned European ancestry in the previous step, then the subject's genotypes were imputed using the European population from 1000 Genomes as the reference population.

Non-Ancestry-Specific Reference Population.

To provide a comparison of the accuracy of the GRS using a reference population that is not ancestry specific, the subject's genotypes were imputed using European population from 1000 Genomes as the reference population.

Calculation of GRS.

An analysis comparing cases of lactose tolerance at the rs4988235, rs41380347, and rs145946881 imputed genotypes was performed using both the ancestry-specific reference population and the non-ancestry-specific reference population for comparison. A subject's lactose tolerance score was calculated based on the fraction of non-reference alleles.

Results.

Table 47 illustrates a pipeline using a single reference population where ancestry is not considered, whereas Table 48 illustrates an ancestry-specific pipeline where the pipeline is specific to the ancestry of the individual. It is known that 98% of the population in Asian countries that are close to the equator are unable to digest lactose and are lactose-intolerant. This is accurately reflected in Table 48 where the ancestry-specific pipeline predicted that 100% of the individuals with East Asian ancestry living in Singapore are lactose intolerant. In contrast, Table 47 illustrates when Asian ancestry is not considered, 57% of the East Asian individuals were predicted to be lactose tolerant which does not match what is known. “EUR” refers to Europeans, and “EAS” refers to East Asian.

TABLE 47
Pipeline using single reference population
in which ancestry was not considered

| | | Individuals with | Individuals |
|---|---|---|---|
| | Lactose | European ancestry | with East Asian |
| Score | Tolerant | (n = 193) | ancestry (n = 1476) |
| 0 | No | 14% (27/193) | 44% (648/1476) |
| 0.17 | Yes | 42% (81/193) | 46% (672/1476) |
| 0.33 | Yes | 44% (85/193) | 11% (156/1476) |
| 0.50 | Yes | 0% (0/193) | 0% (0/1476) |
| 0.67 | Yes | 0% (0/193) | 0% (0/1476) |
| 0.83 | Yes | 0% (0/193) | 0% (0/1476) |
| 1.00 | Yes | 0% (0/193) | 0% (0/1476) |

TABLE 48
Ancestry-specific pipeline (pipeline specific to
ancestry of individual, where EUR is used for EUR
individuals and EAS is used for EAS individuals).

| | | Individuals with | Individuals with East |
|---|---|---|---|
| | Lactose | European ancestry | Asian ancestry % |
| Score | Tolerant | (n = 193) | (n = 1476) |
| 0 | No | 14% (27/193) | 100% (1476/1476) |
| 0.17 | Yes | 42% (81/193) | 0% (0/1476) |
| 0.33 | Yes | 44% (85/193) | 0% (0/1476) |
| 0.50 | Yes | 0% (0/193) | 0% (0/1476) |
| 0.67 | Yes | 0% (0/193) | 0% (0/1476) |
| 0.83 | Yes | 0% (0/193) | 0% (0/1476) |
| 1.00 | Yes | 0% (0/193) | 0% (0/1476) |

Disclosed herein are several gene symbols representing human genes of interest. Table 49 provides a list of genes and corresponding gene names.

TABLE 49

| Gene Symbol | Full Gene Name |
|---|---|
| AQP3 | aquaporin 3 |
| NQO1 | NAD(P)H dehydrogenase[quinone] 1 |
| SOD2 | Superoxide dismutase II |
| NFE2L2 | Nuclear factor erythroid 2-related factor 2 |
| GPX1 | Glutathione peroxidase 1 |
| CAT | Catalase |
| MMP | Matrix Metallopeptidase |
| MMP-3 | Matrix Metallopeptidase 3 |
| MMP-1 | Matrix Metallopeptidase 1 |
| Catalase | Catalase |
| LOC157273 | Uncharacterized LOC157273 |
| SGOL1 | Shugoshin 1 |
| TBC1D22B | TBC1 Domain Family Member 22B |
| FST | Follistatin |
| MIR4432 | MicroRNA 4432 |
| RNASEH2C | Ribonuclease H2 Subunit C |
| TGFB2 | Transforming Growth Factor Beta 2 |
| SLC24A5 | Solute Carrier Family 24 Member 5 |
| SLC45A2 | Solute Carrier Family 24 Member 2 |
| BCN2 | Basonuclin 2 |
| MC1R | Melanocortin 1 Receptor |
| C16orf55 | Spermatogenesis Associated 33 |
| SPATA33 | Spermatogenesis Associated 33 |
| NAT2 | N-Acetyltransferase 2 |
| SEC5L1 | Exocyst Complex Component 2 |
| IRF4 | Interferon Regulatory Factor 4 |
| TYR | Tyrosinase |
| ASIP | Agouti Signaling Protein |
| RALY | RALY Heterogeneous Nuclear Ribonucleoprotein |
| EDEM1 | ER Degradation Enhancing Alpha-Mannosidase |
| | Like Protein 1 |
| NTM | Neurotrimin |
| FBXO40 | F-Box Protein 40 |
| STXBP5L | Syntaxin Binding Protein 5 Like |
| FANCA | FA Complementation Group A |
| ID4 - RPL29P17 | Membrane Bound O-Acyltransferase Domain |
| (MB0AT1) | Containing 1 |
| DDB2 | Damage Specific DNA Binding Protein 2 |
| C11orf49 | Chromosome 11 Open Reading Frame 49 |
| SELL | Selectin L |
| ERI1 | Exoribonuclease 1 |
| MFHAS1 | Malignant Fibrous Histiocytoma Amplified |
| | Sequence 1 |
| MIR597 | MicroRNA 597 |
| MIR4660 | MicroRNA 4660 |
| PPP1R3B | Protein Phosphatase 1 Regulatory |
| | Subunit 3B |
| U6 | RNA, U6 Small Nuclear 1 |
| TNKS | Tankyrase |
| BC017578 | ENSG00000248538 Gene |
| (AC022784.1) | |
| PAPSS2 | 3′-Phosphoadenosine 5′-Phosphosulfate |
| | Synthase 2 |
| C18orf2 | Charged Multivesicular Body Protein 1B |
| DNAPTP6 | Spermatogenesis Associated Serine Rich |
| | 2 Like |
| TMEM18 | Transmembrane Protein 18 |
| LEP | Leptin |
| MC4R | Melanocortin 4 Receptor |
| TSHR | Thyroid Stimulating Hormone Receptor |
| ACSL1 | Acyl-CoA Synthetase Long Chain Family |
| | Member 1 |
| PRDM1 | PR/SET Domain 1 |
| DBX1 | Developing Brain Homeobox 1 |
| GRIN3A | Glutamate Ionotropic Receptor NMDA Type |
| | Subunit 3A |
| ESRRB | Estrogen Related Receptor Beta |
| ZIC4 | Zic Family Member 4 |
| FTO | FTO Alpha-Ketoglutarate Dependent |
| | Dioxygenase |
| KCTD15 | Potassium Channel Tetramerization Domain |
| | Containing 15 |
| CHST8 | Carbohydrate Sulfotransferase 8 |
| PPARGC1A | PPARG Coactivator 1 Alpha |
| PPAR-a | Peroxisome Proliferator Activated |
| | Receptor Alpha |
| CDH13 | Cadherin 13 |
| KLKB1 | Kallikrein B1 |
| F12 | Coagulation Factor XII |
| CETP | Cholesteryl Ester Transfer Protein |
| APOE | Apolipoprotein E |
| APOC1 | Apolipoprotein C1 |
| RBPMS | RNA Binding Protein, MRNA Processing Factor |
| PIWIL1 | Piwi Like RNA-Mediated Gene Silencing 1 |
| OR6N2 | Olfactory Receptor Family 6 Subfamily N |
| | Member 2 |
| ERBB4 | Erb-B2 Receptor Tyrosine Kinase 4 |
| CREB1 | CAMP Responsive Element Binding Protein 1 |
| MAP2 | Microtubule Associated Protein 2 |
| TRHR | Thyrotropin Releasing Hormone Receptor |
| DARC | Atypical Chemokine Receptor 1 (Duffy |
| | Blood Group) |
| GLYAT | Glycine-N-Acyltransferase |
| FADS1 | Fatty Acid Desaturase 1 |
| FADS2 | Fatty Acid Desaturase 2 |
| CD163L1 | CD163 Molecule Like 1 |
| CD163 | CD163 Molecule |
| ABO | ABO, Alpha 1-3-N-Acetylgalactosaminyl- |
| | transferase And Alpha |
| | 1-3-Galactosyltransferase |
| CRP | C-Reactive Protein |
| CADM3 | Cell Adhesion Molecule 3 |
| IGF-II | Insulin Like Growth Factor 2 |
| MLCK | Myosin Light Chain Kinase |
| ACTN3 | Actinin Alpha 3 |
| IL-6 | Interleukin 6 |
| COL5A1 | Collagen Type V Alpha 1 Chain |
| HCP5 | HLA Complex P5 |
| HCG26 | HLA Complex Group 26 |
| MICB | MHC Class I Polypeptide-Related Sequence B |
| ATP6V1G2 | ATPase H+ Transporting V1 Subunit G2 |
| DDX39B | DExD-Box Helicase 39B |
| LOC101060363 | Zinc Finger And BTB Domain Containing 40 |
| (ZBTB40) | |
| LOC105376856 | Uncharacterized LOC105376856 |
| EN1 | Engrailed Homeobox 1 |
| FLJ42280 | SEM1 26S Proteasome Complex Subunit |
| (C7orf76) | |
| COLEC10 | Collectin Subfamily Member 10 |
| WNT16 | Wnt Family Member 16 |
| ESR1 | Estrogen Receptor 1 |
| ATP6V1G1 | ATPase H+ Transporting V1 Subunit G1 |
| HAO1 | Hydroxyacid Oxidase 1 |
| RSPO2 | R-Spondin 2 |
| EMC2 | ER Membrane Protein Complex Subunit 2 |
| EIF3E | Eukaryotic Translation Initiation Factor |
| | 3 Subunit E |
| CCDC91 | Coiled-Coil Domain Containing 91 |
| PTHLH | Parathyroid Hormone Like Hormone |
| LOC100506393 | Long Intergenic Non-Protein Coding RNA 2398 |
| (LINC02398) | |
| LINC00536 | Long Intergenic Non-Protein Coding RNA 536 |
| EIF3H | Eukaryotic Translation Initiation Factor |
| | 3 Subunit H |
| CDC5L | Cell Division Cycle 5 Like |
| SUPT3H | SPT3 Homolog, SAGA And STAGA Complex |
| | Component |
| MIR4642 | MicroRNA 4642 |
| GC | GC Vitamin D Binding Protein |
| FUT2 | Fucosyltransferase 2 |
| HAAO | 3-Hydroxyanthranilate 3,4-Dioxygenase |
| BCMO1 | Beta-Carotene Oxygenase 1 |
| CASR | Calcium Sensing Receptor |
| TF | Transferrin |
| TFR2 | Transferrin Receptor 2 |
| SCAMP5 | Secretory Carrier Membrane Protein 5 |
| PPCDC | Phosphopantothenoylcysteine Decarboxylase |
| ARSB | Arylsulfatase B |
| DMGDH | Dimethylglycine Dehydrogenase |
| BHMT2 | Betaine--Homocysteine S-Methyltransferase 2 |
| ATP2B1 | ATPase Plasma Membrane Ca2+ |
| | Transporting 1 |
| DCDC5 | Doublecortin Domain Containing 1 |
| GGT1 | Gamma-Glutamyltransferase 1 |
| GGTLC2 | Gamma-Glutamyltransferase Light Chain 2 |
| MYL2 | Myosin Light Chain 2 |
| C12orf27 | HNF1A Antisense RNA 1 |
| HNF1A | HNF1 Homeobox A |
| OAS1 | 2′-5′-Oligoadenylate Synthetase 1 |
| C14orf73 | Exocyst Complex Component 3 Like 4 |
| ZNF827 | Zinc Finger Protein 827 |
| RORA | RAR Related Orphan Receptor A |
| G6PC2 | Glucose-6-Phosphatase Catalytic |
| | Subunit 2 |
| MTNR1B | Melatonin Receptor 1B |
| GCK | Glucokinase |
| ADCY5 | Adenylate Cyclase 5 |
| MADD | MAP Kinase Activating Death Domain |
| ADRA2A | Adrenoceptor Alpha 2A |
| MRPL33 | Mitochondrial Ribosomal Protein L33 |
| CACNA2D3 | Calcium Voltage-Gated Channel Auxiliary |
| | Subunit Alpha2delta 3 |
| NEDD4L | NEDD4 Like E3 Ubiquitin Protein Ligase |
| AC105008.1 | RNA, U1 Small Nuclear 35, Pseudogene |
| (RNU1-35P) | |
| P2RY2 | Purinergic Receptor P2Y2 |
| RP11-479A21.1 | BTB Domain Containing 7 Pseudogene 2 |
| (BTBD7P2) | |
| MTUS2 | Microtubule Associated Scaffold Protein 2 |
| PIBF1 | Progesterone Immunomodulatory Binding Factor 1 |
| IRAK1BP1 | Interleukin 1 Receptor Associated Kinase 1 |
| | Binding Protein 1 |
| PRMT6 | Protein Arginine Methyltransferase 6 |
| CDCA7 | Cell Division Cycle Associated 7 |
| NOTCH4 | Notch Receptor 4 |
| BTNL2 | Butyrophilin Like 2 |
| HLA-DRA | Major Histocompatibility Complex, Class II, |
| | DR Alpha |
| ARSJ | Arylsulfatase Family Member J |
| CSMD1 | CUB And Sushi Multiple Domains 1 |
| HLA-DRB1 | Major Histocompatibility Complex, |
| | Class II, DR Beta 1 |
| HLA-DQA1 | Major Histocompatibility Complex, |
| | Class II, DQ Alpha 1 |
| HLA-DQB1 | Major Histocompatibility Complex, |
| | Class II, DQ Beta 1 |
| HLA-DQA2 | Major Histocompatibility Complex, |
| | Class II, DQ Alpha 2 |
| HCG27 | HLA Complex Group 27 |
| ADGB | Androglobin |
| RPS15P9 | Ribosomal Protein S15 Pseudogene 9 |
| MUM1 | PWWP Domain Containing 3A, DNA Repair Factor |
| RYR1 | Ryanodine Receptor 1 |
| LINC00992 | Long Intergenic Non-Protein Coding RNA 992 |
| LOC100129526 | Protein Tyrosine Phosphatase Receptor |
| | Type D Pseudogene |
| FAM118A | Family With Sequence Similarity 118 |
| | Member A |
| SMC1B | Structural Maintenance Of Chromosomes IB |
| LEPR | Leptin Receptor |
| FGF21 | Fibroblast Growth Factor 21 |
| ZPR1 | ZPR1 Zinc Finger |
| TANK | TRAF Family Member Associated NFKB |
| | Activator |
| FNBP1 | Formin Binding Protein 1 |
| RNU6-229P | RNA, U6 Small Nuclear 229, Pseudogene |
| LOC105375346 | Uncharacterized LOC105375346 |
| ARGFX | Arginine-Fifty Homeobox |
| BEND3 | BEN Domain Containing 3 |
| FCER1A | Fc Fragment Of IgE Receptor Ia |
| LRRC32 | Leucine Rich Repeat Containing 32 |
| C11orf30 | EMSY Transcriptional Repressor, BRCA2 |
| | Interacting |
| IL13 | Interleukin 13 |
| OR10J3 | Olfactory Receptor Family 10 Subfamily J |
| | Member 3 |
| HLA-A | Major Histocompatibility Complex, |
| | Class I, A |
| STAT6 | Signal Transducer And Activator Of |
| | Transcription 6 |
| TSLP | Thymic Stromal Lymphopoietin |
| SLC25A46 | Solute Carrier Family 25 Member 46 |
| WDR36 | WD Repeat Domain 36 |
| CAMK4 | Calcium/Calmodulin Dependent Protein Kinase IV |
| LOC730217 | Long Intergenic Non-Protein Coding RNA 1550 |
| (LINC01550) | |
| OPRK1 | Opioid Receptor Kappa 1 |
| OR6X1 | Olfactory Receptor Family 6 |
| | Subfamily X Member 1 |
| DOCK10 | Dedicator Of Cytokinesis 10 |
| Cap S | cathepsin S |
| IL4 | Interleukin 4 |
| FASTKD2 | FAST Kinase Domains 2 |
| MIR3130-1 | MicroRNA 3130-1 |
| MIR3130-2 | MicroRNA 3130-2 |
| SPOCK3 | SPARC (Osteonectin), Cwcv And Kazal Like |
| | Domains Proteoglycan 3 |
| ANXA10 | Annexin A10 |
| ISL1 | ISL LIM Homeobox 1 |
| PARP8 | Poly(ADP-Ribose) Polymerase Family Member 8 |
| BAIAP2 | BAI1 Associated Protein 2 |
| HS3ST4 | Heparan Sulfate-Glucosamine |
| | 3-Sulfotransferase 4 |
| C16orf82 | Chromosome 16 Open Reading Frame 82 |
| AJAP1 | Adherens Junctions Associated Protein 1 |
| C1orf174 | Chromosome 1 Open Reading Frame 174 |
| PTPRD | Protein Tyrosine Phosphatase Receptor |
| | Type D |
| LOC646114 | ENSG00000285784 |
| (AL353595.1) | |
| LOC100049717 | ENSG00000285784 |
| (AL353595.1) | |
| FAIM2 | Fas Apoptotic Inhibitory Molecule 2 |
| AQP2 | Aquaporin 2 |
| TXNL1 | Thioredoxin Like 1 |
| WDR7 | WD Repeat Domain 7 |
| CDH10 | Cadherin 10 |
| MSNL1 | Moesin Pseudogene 1 |
| GRIK2 | Glutamate Ionotropic Receptor Kainate |
| | Type Subunit 2 |
| HACE1 | HECT Domain And Ankyrin Repeat Containing E3 |
| | Ubiquitin Protein Ligase 1 |
| DACH1 | Dachshund Family Transcription Factor 1 |
| MZT1 | Mitotic Spindle Organizing Protein 1 |
| DLGAP1 | DLG Associated Protein 1 |
| AR | Androgen Receptor |
| PAX1 | Paired Box 1 |
| FOXA2 | Forkhead Box A2 |
| ULBP3 | UL16 Binding Protein 3 |
| ULBP6 | Retinoic Acid Early Transcript 1L |
| EDA2R | Ectodysplasin A2 Receptor |
| BQ013595 | Ribosomal Protein L41 Pseudogene 1 |
| (RPL41P1) | |
| BE789145 | Ribosomal Protein L41 Pseudogene 1 |
| (RPL41P1) | |
| WNT10A | Wnt Family Member 10A |
| FFAR4 | Free Fatty Acid Receptor 4 |
| TTR | Transthyretin |
| HLA-C | Major Histocompatibility Complex, |
| | Class I, C |

While preferred embodiments of the methods, media, and systems disclosed herein have been shown and described herein, such embodiments are provided by way of example only. Numerous variations, changes, and substitutions may be done without departing from the methods, media, and systems disclosed herein. It should be understood that various alternatives to the embodiments of the methods, media, and system disclosed herein may be employed in practicing the inventive concepts disclosed herein. It is intended that the following claims define the scope of the methods, media, and systems that methods and structures within the scope of these claims and their equivalents be covered thereby.

What is claimed is:

1. A computer-implemented method comprising:
a) assigning an ancestry of an individual using a distance-based or a models-based computer program to analyze a genotype of the individual, the genotype comprising one or more individual-specific genetic variants; and
b) detecting in the genotype of the individual, an ancestry-specific variant associated with a specific phenotypic trait, the ancestry-specific variant corresponding to:
i) an individual-specific genetic variant detectable in the genotype of the individual; or
ii) a genetic variant in linkage disequilibrium (LD) with the individual-specific genetic variant as determined by imputing the individual-specific variant missing from ancestry-specific phased haplotypes determined using a reference group of individuals that has the same ancestry as the individual; and
c) calculating a genetic risk score (GRS) for the individual based on the ancestry-specific variant detected in (b), wherein the GRS is indicative of a likelihood that the individual has, or will develop, the specific phenotypic trait.

2. The computer-implemented method of claim 1, wherein the ancestry-specific genetic variant and the individual-specific genetic variant is selected from the group consisting of a single nucleotide variant (SNV), a copy number variant (CNV), and an indel.

3. The computer-implemented method of claim 1, wherein imputing in step (ii) comprises:
a) phasing unphased genotype data from the individual to generate ancestry-specific phased haplotypes based on the ancestry of the individual; and
b) imputing individual-specific genotypes not present in the ancestry-specific phased haplotypes using phased haplotype data from the reference group that has the same ancestry as the individual to select the genetic variant in LD with the individual-specific genetic variant.

4. The computer-implemented method of claim 1, wherein the LD is defined by a D′ value comprising at least about 0.80 or a r2 value comprising at least 0.80.

5. The computer-implemented method of claim 1, wherein the specific trait comprises a nutritional trait, a clinical trait, a subclinical trait, a physical exercise trait, a skin trait, an allergy trait, or a mental trait, or combination thereof.

6. The computer-implemented method of claim 1, wherein the genotype of the individual is obtained by subjecting, or having subjected, genetic material obtained from the individual to a genotyping assay.

7. The computer-implemented method of claim 6, wherein the genotyping assay comprises a deoxyribonucleic acid (DNA) array, ribonucleic acid (RNA) array, sequencing assay, or a combination thereof.

8. The computer-implemented method of claim 1, wherein the distance-based computer program is principle component analysis, and wherein the models-based computer program is a maximum likelihood or a Bayesian method.

9. The computer-implemented method of claim 1, wherein the GRS for the individual based on the ancestry-specific variant is more accurate than a corresponding GRS of the individual based on a variant that is not ancestry-specific.

10. The computer-implemented method of claim 1, further comprising providing a notification comprising the GRS for the specific phenotypic trait of the individual.

11. The computer-implemented method of claim 10, wherein the notification further comprises a behavior recommendation to the individual based on the GRS for the specific phenotypic trait.

12. The computer-implemented method of claim 11, wherein the behavioral modification related to the specific phenotypic trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption.

13. A system comprising:
a computing device comprising at least one processor, a memory, and a software program including instructions executable by at least one processor to assess a likelihood that an individual has, or will develop, a specific phenotypic trait, the instructions comprising the steps of:
a) assigning an ancestry of an individual using a distance-based or a models-based computer program to analyze a genotype of the individual, the genotype comprising one or more individual-specific genetic variants; and
b) detecting in the genotype of the individual, an ancestry-specific variant associated with a specific phenotypic trait, the ancestry-specific variant corresponding to:
i) an individual-specific genetic variant detectable in the genotype of the individual; or
ii) a genetic variant in linkage disequilibrium (LD) with the individual-specific genetic variant as determined by imputing the individual-specific variant missing from ancestry-specific phased haplotypes determined using a reference group of individuals that has the same ancestry as the individual; and
c) calculating a genetic risk score (GRS) for the individual based on the ancestry-specific variant detected in (b), wherein the GRS is indicative of a likelihood that the individual has, or will develop, the specific phenotypic trait.

14. The system of claim 13, wherein the ancestry-specific genetic variant and the individual specific genetic variant is selected from the group consisting of a single nucleotide variant (SNV), a copy number variant (CNV), and an indel.

15. The system of claim 13, wherein imputing in step (2) comprises:
a) phasing unphased genotype data from the individual to generate ancestry-specific phased haplotypes based on the ancestry of the individual; and
b) imputing individual-specific genotypes not present in the ancestry-specific phased haplotypes using phased haplotype data from the reference group that has the same ancestry as the individual to select the genetic variant in LD with the individual-specific genetic variant.

16. The system of claim 13, wherein the LD is defined by a D′ value comprising at least about 0.80 or a r2 value comprising at least 0.80.

17. The system of claim 13, wherein the specific trait comprises a nutritional trait, a clinical trait, a subclinical trait, a physical exercise trait, a skin trait, an allergy trait, or a mental trait.

18. The system of claim 13, further comprising a genotyping assay.

19. The system of claim 18, wherein the genotyping assay comprises a deoxyribonucleic acid (DNA) array, ribonucleic acid (RNA) array, sequencing assay, or a combination thereof.

20. The system of claim 13, wherein the distance-based computer program is principle component analysis, and wherein the models-based computer program is a maximum likelihood or a Bayesian method.

21. The system of claim 13, wherein the GRS for the individual based on the ancestry-specific variant is more accurate than a corresponding GRS of the individual based on a variant that is not ancestry-specific.

22. The system of claim 13, further comprising a reporting module configured to generate a report comprising the GRS of the individual for the specific phenotypic trait.

23. The system of claim 13, further comprising an output module configured to display the report to the individual.

24. The system of claim 23, wherein the report comprises the risk that the individual has, or will develop, the specific trait.

25. The system of claim 23, wherein the report further comprises a recommendation of a behavior recommendation to the individual based on the GRS for the specific phenotypic trait.

26. The system of claim 25, wherein the behavioral modification related to the specific phenotypic trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption.

27. Use of the system of claim 13, for recommending a behavior modification or a product to the individual, based on the GRS calculated in (c).

28. A non-transitory computer readable storage medium, comprising computer-executable code configured to cause at least one processor to perform steps provided in claim 1.

29. A computer-implemented method of determining a likelihood that an individual has, or will develop, a specific phenotypic trait based on the ancestry of the individual, the method comprising:
a) assigning an ancestry of the individual by using a distance-based or a models-based computer program to analyze a genotype of the individual, the genotype comprising one or more individual-specific genetic variants;
b) selecting, from a trait-associated variants database comprising ancestry-specific genetic variants derived from subjects with the same ancestry as the individual (subject group), one or more ancestry-specific genetic variants based, at least in part, on the ancestry of the individual, wherein each of the one or more ancestry-specific genetic variants correspond to:
i) an individual-specific genetic variant of the one or more individual-specific genetic variants, or
ii) a predetermined genetic variant in a linkage disequilibrium (LD) with an individual-specific genetic variant of the one or more individual-specific genetic variants in a subject population with the same ancestry as the individual, wherein the predetermined genetic variant is predetermined by:
1) phasing unphased genotype data from the individual to generate individual-specific phased haplotypes based on the ancestry of the individual;
2) imputing individual-specific genotypes not present in the phased individual-specific phased haplotypes using phased haplotype data from a reference group that has the same ancestry as the individual; and
3) selecting a genetic variant from the imputed individual-specific genotypes that matches with the individual-specific genetic variant associated with a likelihood that the individual has, or will develop, a specific phenotypic trait and corresponding to the one or more ancestry specific variants,
wherein each of the one or more ancestry-specific genetic variants and each of the one or more individual specific genetic variants comprise one or more units of risk; and
4) calculating a genetic risk score for the individual based on the selected one or more ancestry-specific genetic variants, wherein the genetic risk score is indicative of the likelihood that the individual has, or will develop the specific phenotypic trait.

30. The method of claim 29, wherein the one or more ancestry-specific genetic variants, the one or more individual-specific genetic variants, and the genetic variants in LD with the one or more individual-specific genetic variants comprise a Single Nucleotide Variant (SNV), an indel, and/or a Copy Number Variant (CNV).

31. The method of claim 29, wherein the one or more units of risk of the SNV comprises a risk allele; the one or more units of risk of the indel comprises a presence (I) or an absence (D) of the nucleotide; and the one or more units of risk of the CNV comprises an insertion or a deletion of a nucleic acid sequence.

32. The method of claim 29, further comprising providing a notification to the individual comprising the risk that the individual has, or will develop, the specific phenotypic trait.

33. The method of claim 29, wherein the specific phenotypic trait comprises a nutritional trait, a clinical trait, a subclinical trait, a physical exercise trait, a skin trait, a hair trait, an allergy trait, or a mental trait.

34. The method of claim 33, wherein the notification further comprises a recommendation for a behavior modification related to the specific phenotypic trait.

35. The method of claim 34, wherein the behavior modification related to the specific phenotypic trait comprises increasing, reducing, or avoiding an activity comprising performance of a physical exercise, ingestion of a drug, vitamin, or supplement, exposure to a product, usage of a product, a diet modification, sleep modification, alcohol consumption, or caffeine consumption.

36. The method of claim 29, wherein the distance-based computer program is principle component analysis, and wherein the models-based computer program is a maximum likelihood or a Bayesian method.

37. A wellness reporting system comprising:
a) a computing device comprising at least one processor, a memory, and a software program including instructions executable by at least one processor to assess a likelihood that an individual has, or will develop, a specific phenotypic trait, the instructions comprising the steps of:
i) assigning an ancestry of the individual by using a distance-based or a models-based computer program to analyze a genotype of the individual, the genotype comprising one or more individual-specific genetic variants;
ii) selecting, from a trait-associated variants database comprising ancestry-specific genetic variants derived from subjects with the same ancestry as the individual (subject group), one or more ancestry-specific genetic variants based, at least in part, on the ancestry of the individual, wherein each of the one or more ancestry-specific genetic variants correspond to:
1) an individual-specific genetic variant of the one or more individual-specific genetic variants, or
2) a predetermined genetic variant in a linkage disequilibrium (LD) with an individual-specific genetic variant of the one or more individual-specific genetic variants in a subject population with the same ancestry as the individual,
wherein the predetermined genetic variant is predetermined by:
a) phasing unphased genotype data from the individual to generate individual-specific phased haplotypes based on the ancestry of the individual;
b) imputing individual-specific genotypes not present in the phased individual-specific phased haplotypes using phased haplotype data from a reference group that has the same ancestry as the individual; and
c) selecting a genetic variant from the imputed individual-specific genotypes that matches with the individual-specific genetic variant associated with a likelihood that the individual has, or will develop, a specific phenotypic trait and corresponding to the one or more ancestry specific variants,
wherein each of the one or more ancestry-specific genetic variants and each of the one or more individual specific genetic variants comprise one or more units of risk; and
d) calculating a genetic risk score for the individual based on the selected one or more ancestry-specific genetic variants, wherein the genetic risk score is indicative of the likelihood that the individual has, or will develop the specific phenotypic trait;
b) a reporting module configured to generate a report comprising the genetic risk score of the individual for the specific phenotypic trait; and
c) an output module configured to display the report to the individual.

38. The system of claim 37, wherein the one or more ancestry-specific genetic variants, the one or more individual-specific genetic variants, and the genetic variants in LD with the one or more individual-specific genetic variants comprise a Single Nucleotide Variant (SNV), an indel, and/or a Copy Number Variant (CNV).

39. The system of claim 38, wherein the one or more units of risk of the SNV comprises a risk allele; the one or more units of risk of the indel comprises an insertion (I) or a deletion (D) of the nucleotide; and the one or more units of risk of the CNV comprises an insertion or a deletion of a nucleic acid sequence.

40. The system of claim 37, wherein the report further comprises a recommendation for a behavior modification related to the specific phenotypic trait.

41. The system of claim 37, wherein the specific phenotypic trait comprises a nutritional trait, a clinical trait, a subclinical trait, a physical exercise trait, a skin trait, a hair trait, an allergy trait, or a mental trait.

42. The system of claim 37, further comprising a personal electronic device with an application configured to communicate with the output module via a computer network to access the report.

43. The system of claim 37, wherein the distance-based computer program is principle component analysis, and wherein the models-based computer program is a maximum likelihood or a Bayesian method.

44. A non-transitory computer readable storage medium, comprising computer-executable code configured to cause at least one processor to perform steps comprising:
a) assigning an ancestry of the individual by using a distance-based or a models-based computer program to analyze a genotype of the individual, the genotype comprising one or more individual-specific genetic variants;
b) selecting, from a trait-associated variants database comprising ancestry-specific genetic variants derived from subjects with the same ancestry as the individual (subject group), one or more ancestry-specific genetic variants based, at least in part, on the ancestry of the individual, wherein each of the one or more ancestry-specific genetic variants correspond to:
i) an individual-specific genetic variant of the one or more individual-specific genetic variants, or
ii) a predetermined genetic variant in a linkage disequilibrium (LD) with an individual-specific genetic variant of the one or more individual-specific genetic variants in a subject population with the same ancestry as the individual, wherein the predetermined genetic variant is predetermined by:
1) providing unphased genotype data from the individual;
2) phasing the unphased genotype data to generate individual-specific phased haplotypes based on the ancestry of the individual;
3) imputing individual-specific genotypes not present in the phased individual-specific phased haplotypes using phased haplotype data from a reference group that has the same ancestry as the individual; and
4) selecting a genetic variant from the imputed individual-specific genotypes that matches with the individual-specific genetic variant associated with a likelihood that the individual has, or will develop, a specific phenotypic trait; and
c) calculating a genetic risk score for the individual based on the selected one or more ancestry-specific genetic variants, wherein the genetic risk score is indicative of the likelihood that the individual has, or will develop the specific phenotypic trait.

45. The medium of claim 44, wherein the one or more ancestry-specific genetic variants, the one or more individual-specific genetic variants, and the genetic variants in LD with the one or more individual-specific genetic variants comprise a Single Nucleotide Variant (SNV), an indel, and/or a Copy Number Variant (CNV).

46. The medium of claim 45, wherein each of the one or more ancestry-specific genetic variants and each of the individual specific genetic variants comprises one or more units of risk, and wherein the one or more units of risk of the SNV comprises a risk allele; the one or more units of risk of the indel comprises an insertion (I) or a deletion (D) of a nucleotide; and the one or more units of risk of the CNV comprises an insertion or a deletion of a nucleic acid sequence.

47. The medium of claim 46, wherein the steps further comprise providing a notification to the individual comprising the likelihood that the individual has, or will develop, the specific phenotypic trait.

48. The medium of claim 47, wherein the specific phenotypic trait comprises a nutritional trait, a clinical trait, a subclinical trait, a physical exercise trait, a skin trait, a hair trait, an allergy trait, or a mental trait.
